(19) United States
(12) Patent Application Publication
(10) Pub. No.: US 2023/0302036 A1
(43) Pub. Date: Sep. 28, 2023

(54) COMPOSITIONS FOR TREATMENT OF SPINAL MUSCULAR ATROPHY

(71) Applicants: CONSEJO NACIONAL DE INVESTIGACIONES CIENTIFICAS Y TECNICAS, Buenos Aires (AR); UNIVERSIDAD DE BUENOS AIRES, Buenos Aires (AR); COLD SPRING HARBOR LABORATORY, Cold Spring Harbor, NY (US)

(72) Inventors: Luciano E. MARASCO, Buenos Aires (AR); Alberto R. KORNBLIHTT, Buenos Aires (AR); Adrian KRAINER, New York, NY (US); Jose STIGLIANO, Buenos Aires (AR)

(21) Appl. No.: 18/005,514

(22) PCT Filed: Jul. 13, 2021
(86) PCT No.: PCT/US2021/041466

§ 371 Date: Jan. 13, 2023

Related U.S. Application Data
(60) Provisional application No. 63/051,279, filed on Jul. 13, 2020.

(51) Int. Cl. A61K 31/7125; A61K 31/711; C12N 15/113

(52) U.S. Cl. CPC A61K 31/7125; A61K 31/711; C12N 15/113; C12N 2310/11; C12N 2320/31

(57) ABSTRACT

The present disclosure relates compositions and methods to treat neuromuscular diseases and disorders, e.g., spinal muscular atrophy (SMA), characterized by the presence of a splicing silencer located in SMN2 intron 7 pre-mRNA, comprising coadministering a therapeutically effective amount of an antisense oligonucleotide (ASO) complementary to a nucleotide sequence within intron 7 of human SMN2 pre-mRNA; and a subclinical dose of a histone deacetylate inhibitor, e.g., valproic acid, trichostatin A, or a combination thereof.

### CROSS-REFERENCE TO RELATED APPLICATIONS AND INCORPORATION BY REFERENCE

This PCT application claims the priority benefit of U.S. Provisional Application No. 63/051,279, filed on Jul. 13, 2020, which is herein incorporated by reference in its entirety.

### STATEMENT REGARDING FEDERALLY-SPONSORED RESEARCH AND DEVELOPMENT

Part of the work performed during development of this invention utilized U.S. Government funds. This invention was made with government support under GM42699 awarded by the National Institutes of Health. The U.S. Government has certain rights in this invention.

### REFERENCE TO SEQUENCE LISTING SUBMITTED ELECTRONICALLY

The content of the electronically submitted sequence listing in ASCII text file (Name 3181_007PC01_Seqlisting_ST25.txt; Size: 16,894 bytes; and Date of Creation: Jul. 12, 2021) filed with the application is incorporated herein by reference in its entirety.

### FIELD OF DISCLOSURE

The present disclosure relates to compositions for the treatment of spinal muscular atrophy comprising an antisense therapy agent, e.g., an antisense oligonucleotide (ASO), and a histone deacetylase inhibitor, e.g., valproic acid, at a subclinical dose and optionally additional adjuvants.

### BACKGROUND

Spinal muscular atrophy (SMA) is an autosomal recessive neuromuscular disease that affects approximately 1 in 10,000 newborns, caused by mutations in motor neuron 1 gene (SMN1) in chromosome 5. Reduced expression of functional survival motor neuron (SMN) protein is responsible for SMA. Humans have a paralogous gene, SMN2, on the same chromosome, that also encodes SMN, but at levels that are too low to fully compensate for SMN1 loss of function. The shortcoming of SMN2 is mostly due to one of the 11 nucleotide sequence differences with its paralogue that causes poor inclusion of exon 7 (E7) into SMN2 mature mRNA through alternative splicing. Due to E7 skipping, SMN2 predominantly encodes a truncated, unstable SMN protein, and only a small amount of full-length, functional SMN.

SMA was the leading genetic cause of infant mortality until a splicing-correcting antisense oligonucleotide therapy was approved for clinical use at the end of 2016. A 2′-O-(2-methoxyethyl) (MOE) phosphorothioate-modified antisense-oligonucleotide drug known as nusinersen targets a splicing silencer located in SMN2 intron 7 pre-mRNA and, by blocking the binding of the splicing repressors hnRNPA1 and A2, promotes higher E7 inclusion, resulting in higher SMN protein levels (Hua et al., Am. J. of Hum. Genet. 82, 834-848, 2008). Nusinersen was the first drug approved for SMA therapy, and also the first-splicing-corrective drug. Three years after its approval, approximately 10,000 SMA patients have been treated with it worldwide. Although nusinersen is administered to patients by lumbar puncture, to reach motor neurons through cerebrospinal fluid (CSF), systemic administration robustly rescues severe symptoms in an SMA mouse model (Hua et al., Nature 478, 123-126, 2011; Hua et al., Genes Dev. 29, 288-297, 2015). This reflects the fact that SMN expression is ubiquitous, and underscores the importance of restoring adequate SMN levels in peripheral tissues. While being effective in increasing motor function in some patients, nusinersen is not effective treating all SMA patients. Moreover, like other antisense drugs, nusinersen treatment is associated with increased risk of abnormalities in blood clotting, reduction in platelets, kidney damage as well as other side effects (e.g., increased risk of infection and scoliosis). Thus, it is an objective of the present disclosure to provide improved compositions and methods for treating SMA.

### SUMMARY

The present disclosure provides a method for treating a neuromuscular disease or disorder comprising administering a therapeutically effective amount of a composition to a subject in need thereof wherein the composition comprises (i) an antisense oligonucleotide (ASO) complementary to a nucleotide sequence within intron 7 of human SMN2 pre-mRNA; and

(ii) a subclinical dose of a histone deacetylase inhibitor.

Also provided is method for treating a neuromuscular disease or disorder comprising co-administering (i) a therapeutically effective amount of an ASO complementary to a nucleotide sequence within intron 7 of human SMN2 pre-mRNA; and (ii) a subclinical dose of a histone deacetylase inhibitor.

The present disclosure also provides a method to increase the clinical efficacy of an ASO complementary to a nucleotide sequence within intron 7 of human SMN2 pre-mRNA in the treatment of a neuromuscular disease or disorder comprising co-administering (i) a therapeutically effective amount of the ASO to a subject, and (ii) subclinical dose of a histone deacetylase inhibitor.

In some aspects, the neuromuscular disease or disorder is SMA. In some aspects, the ASO is selected from the group consisting of ATTCACTTTCATAATGCTGG (ASO1) (SEQ ID NO:1), TGCTGGCAGACTTAC (SEQ ID NO:58), CATAATGCTGGCAGA (SEQ ID NO:59), TCATAATGCTGGCAG (SEQ ID NO:60), TTCATAATGCTGGCA (SEQ ID NO:61), TTTCATAATGCTGGC (SEQ ID NO:62), TCACTTTCATAATGCTGG (nusinersen) (SEQ ID NO:63), AGTAAGATTCACTTT (SEQ ID NO:64), CTTTCATAATGCTGG (SEQ ID NO:65), TCATAATGCTGG (SEQ ID NO:66), ACTTTCATAATGCTG (SEQ ID NO:67), TTCATAATGCTG (SEQ ID NO:68), CACTTTCATAATGCT (SEQ ID NO:69), TTTCATAATGCT (SEQ ID NO:70), TCACTTTCATAATGC (SEQ ID NO:71), CTTTCATAATGC (SEQ ID NO:72), TTCACTTTCATAATG (SEQ ID NO:73), ACTTTCATAATG (SEQ ID NO:74), ATTCACTTTCATAAT (SEQ ID NO:75), CACTTTCATAAT (SEQ ID NO:76), GATTCACTTTCATAA (SEQ ID NO:77), TCACTTTCATAA (SEQ ID NO:78), TTCACTTTCATA (SEQ ID NO:79), ATTCACTTTCAT (SEQ ID NO:80), or a combination thereof.

In some aspects, the ASO comprises a sequence which is at least about 70%, at least about 75%, at least about 80%, at least about 85%, at least about 90%, at least about 95%, at least about 96%, at least about 97%, at least about 98%, at least about 99%, or about 100% identical to ATTCACTTTCATAATGCTGG (ASO1) (SEQ▫ID▫NO:1), TGCTGGCAGACTTAC (SEQ ID NO:58), CATAATGCTGGCAGA (SEQ ID NO:59), TCATAATGCTGGCAG (SEQ ID NO:60), TTCATAATGCTGGCA (SEQ ID NO:61), TTTCATAATGCTGGC (SEQ ID NO:62), TCACTTTCATAATGCTGG (nusinersen) (SEQ ID NO:63), AGTAAGATTCACTTT (SEQ▫ID▫NO:64), CTTTCATAATGCTGG (SEQ ID NO:65), TCATAATGCTGG (SEQ ID NO:66), ACTTTCATAATGCTG (SEQ ID NO:67), TTCATAATGCTG (SEQ ID NO:68), CACTTTCATAATGCT (SEQ ID NO:69), TTTCATAATGCT (SEQ ID NO:70), TCACTTTCATAATGC (SEQ ID NO:71), CTTTCATAATGC (SEQ ID NO:72), TTCACTTTCATAATG (SEQ ID NO:73), ACTTTCATAATG (SEQ ID NO:74), ATTCACTTTCATAAT (SEQ ID NO:75), CACTTTCATAAT (SEQ ID NO:76), GATTCACTTTCATAA (SEQ ID NO:77), TCACTTTCATAA (SEQ ID NO:78), TTCACTTTCATA (SEQ ID NO:79), or ATTCACTTTCAT (SEQ ID NO:80).

In some aspects, the ASO is an ASO of SEQ ID NO: 1, nusinersen, a variant thereof, a derivative thereof, or a combination thereof. In some aspects, the histone deacetylase inhibitor is valproic acid, trichostatin A, or a combination thereof. In some aspects, (i) the ASO is administered at a dose lower than about 0.20 mg/kg, lower than about 0.19 mg/kg, lower than about 0.18 mg/kg, lower than about 0.17 mg/kg, lower than about 0.16 mg/kg, lower than about 0.15 mg/kg, lower than about 0.14 mg/kg, lower than about 0.13 mg/kg, lower than about 0.12 mg/kg, lower than about 0.11 mg/kg, lower than about 0.1 mg/kg, lower than about 0.09 mg/kg, lower than about 0.08 mg/kg, lower than about 0.07 mg/kg, lower than about 0.06 mg/kg, lower than about 0.05 mg/kg, lower than about 0.04 mg/kg, lower than about 0.03 mg/kg, lower than about 0.02 mg/kg, or lower than about 0.01 mg/kg per dose; and (ii) the histone deacetylase inhibitor is administered at a dose lower than about 15 mg/kg, lower than about 15 mg/kg, lower than about 13 mg/kg, lower than about 12 mg/kg, lower than about 11 mg/kg, lower than about 10 mg/kg, lower than about 9 mg/kg, lower than about 8 mg/kg, lower than about 7 mg/kg, lower than about 6 mg/kg, lower than about 5 mg/kg, lower than about 4 mg/kg, lower than about 3 mg/kg, lower than about 2 mg/kg, lower than about 1 mg/kg per dose.

In some aspects, (i) the ASO is administered at a dose lower than about 12 mg/dose, lower than about 11 mg/dose, lower than about 10 mg/dose, lower than about 9 mg/dose, lower than about 8 mg/dose, lower than about 7 mg/dose, lower than about 6 mg/dose, lower than about 5 mg/dose, lower than about 4 mg/dose, lower than about 3 mg/dose, lower than about 2 mg/dose, or lower than about 1 mg/dose; and (ii) the histone deacetylase inhibitor is administered at a dose lower than about 600 mg/dose, lower than about 550 mg/dose, lower than about 500 mg/dose, lower than about 550 mg/dose, lower than about 500 mg/dose, lower than about 450 mg/dose, lower than about 400 mg/dose, lower than about 350 mg/dose, lower than about 300 mg/dose, lower than about 250 mg/dose, lower than about 200 mg/dose, lower than about 175 mg/dose, lower than about 150 mg/dose, lower than about 125 mg/dose, lower than about 100 mg/dose, lower than about 90 mg/dose, lower than about 80 mg/dose, lower than about 70 mg/dose, lower than about 60 mg/dose, lower than about 50 mg/dose, lower than about 40 mg/dose, lower than about 30 mg/dose, lower than about 20 mg/dose, or lower than about 10 mg/dose.

In some aspects, (i) the ASO is administered at a dose lower than about 12 mg/dose/day, lower than about 11 mg/dose/day, lower than about 10 mg/dose/day, lower than about 9 mg/dose/day, lower than about 8 mg/dose/day, lower than about 7 mg/dose/day, lower than about 6 mg/dose/day, lower than about 5 mg/dose/day, lower than about 4 mg/dose/day, lower than about 3 mg/dose/day, lower than about 2 mg/dose/day, or lower than about 1 mg/dose/day; and (ii) the histone deacetylase inhibitor is administered at a dose lower than about 600 mg/dose/day, lower than about 550 mg/dose/day, lower than about 500 mg/dose/day, lower than about 550 mg/dose/day, lower than about 500 mg/dose/day, lower than about 450 mg/dose/day, lower than about 400 mg/dose/day, lower than about 350 mg/dose/day, lower than about 300 mg/dose/day, lower than about 250 mg/dose/day, lower than about 200 mg/dose/day, lower than about 175 mg/dose/day, lower than about 150 mg/dose/day, lower than about 125 mg/dose/day, lower than about 100 mg/dose/day, lower than about 90 mg/dose/day, lower than about 80 mg/dose/day, lower than about 70 mg/dose/day, lower than about 60 mg/dose/day, lower than about 50 mg/dose/day, lower than about 40 mg/dose/day, lower than about 30 mg/dose/day, lower than about 20 mg/dose/day, or lower than about 10 mg/dose/day.

In some aspects, (i) the ASO is administered at a dose of about 0.20 mg/kg, about 0.19 mg/kg, about 0.18 mg/kg, about 0.17 mg/kg, about 0.16 mg/kg, about 0.15 mg/kg, about 0.14 mg/kg, about 0.13 mg/kg, about 0.12 mg/kg, about 0.11 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, or about 0.01 mg/kg per dose; and (ii) the histone deacetylase inhibitor is administered at a dose about 15 mg/kg, about 15 mg/kg, about 13 mg/kg, about 12 mg/kg, about 11 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg per dose.

In some aspects, (i) the ASO is administered at a dose of about 12 mg/dose, about 11 mg/dose, about 10 mg/dose, about 9 mg/dose, about 8 mg/dose, about 7 mg/dose, about 6 mg/dose, about 5 mg/dose, about 4 mg/dose, about 3 mg/dose, about 2 mg/dose, or about 1 mg/dose; and (ii) the histone deacetylase inhibitor is administered at a dose about 600 mg/dose, about 550 mg/dose, about 500 mg/dose, about 550 mg/dose, about 500 mg/dose, about 450 mg/dose, about 400 mg/dose, about 350 mg/dose, about 300 mg/dose, about 250 mg/dose, about 200 mg/dose, about 175 mg/dose, about 150 mg/dose, about 125 mg/dose, about 100 mg/dose, about 90 mg/dose, about 80 mg/dose, about 70 mg/dose, about 60 mg/dose, about 50 mg/dose, about 40 mg/dose, about 30 mg/dose, about 20 mg/dose, or about 10 mg/dose.

In some aspects, (i) the ASO is administered at a dose of about 12 mg/dose/day, about 11 mg/dose/day, about 10 mg/dose/day, about 9 mg/dose/day, about 8 mg/dose/day, about 7 mg/dose/day, about 6 mg/dose/day, about 5 mg/dose/day, about 4 mg/dose/day, about 3 mg/dose/day, about 2 mg/dose/day, or about 1 mg/dose/day; and (ii) the histone deacetylase inhibitor is administered at a dose about 600 mg/dose/day, about 550 mg/dose/day, about 500 mg/dose/day, about 550 mg/dose/day, about 500 mg/dose/day, about 450 mg/dose/day, about 400 mg/dose/day, about 350 mg/dose/day, about 300 mg/dose/day, about 250 mg/dose/day, about 200 mg/dose/day, about 175 mg/dose/day, about 150 mg/dose/day, about 125 mg/dose/day, about 100 mg/dose/day, about 90 mg/dose/day, about 80 mg/dose/day, about 70 mg/dose/day, about 60 mg/dose/day, about 50 mg/dose/day, about 40 mg/dose/day, about 30 mg/dose/day, about 20 mg/dose/day, or about 10 mg/dose/day.

In some aspects, the administration of the ASO complementary to a nucleotide sequence within intron 7 of human SMN2 pre-mRNA; and the subclinical dose of a histone deacetylate inhibitor results in an increase in inclusion of exon 7 of SMN2, an increase in the expression of SMN2 protein with exon 7, a decrease in the expression of SMN2 protein without exon 7, or any combination thereof. In some aspects, the administration of the ASO complementary to a nucleotide sequence within intron 7 of human SMN2 pre-mRNA; and the subclinical dose of a histone deacetylate inhibitor results in an increase in time of survival, increase in body mass, increase in muscle coordination, improvement in neuromuscular function, or any combination thereof.

In some aspects, the ASO and the subclinical dose of a histone deacetylase inhibitor are administered together. In some aspects, the ASO and the subclinical dose of a histone deacetylase inhibitor are administered separately. In some aspects, the ASO and the subclinical dose of a histone deacetylase inhibitor are administered at the same time. In some aspects, the ASO is administered prior to the administration of the subclinical dose of histone deacetylase inhibitor. In some aspects, the subclinical dose of histone deacetylase inhibitor is administered prior to the administration of the ASO. In some aspects, the ASO is administered intrathecally or intravenously. In some aspects, the histone deacetylase inhibitor is administered orally or intravenously.

In some aspects, the ASO is a gapmer, a mixmer, or a totalmer. In some aspects, the ASO comprises one or more nucleoside analogs. In some aspects, one or more of the nucleoside analogs comprise a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; or bicyclic nucleoside analog. In some aspects, one or more of the nucleoside analogs is a sugar modified nucleoside. In some aspects, the sugar-modified nucleoside is an affinity enhancing 2′ sugar modified nucleoside. In some aspects, one or more of the nucleoside analogs comprises a nucleoside comprising a bicyclic sugar. In some aspects, one or more of the nucleoside analogs comprises an LNA. In some aspects, one or more of the nucleotide analogs is selected from the group consisting of constrained ethyl nucleoside (cEt), 2′,4′-constrained 2′-O-methoxyethyl (cMOE), α-L-LNA, β-D-LNA, 2′-O,4′-C-ethylene-bridged nucleic acids (ENA), amino-LNA, oxy-LNA, thio-LNA, and any combination thereof.

In some aspects, the ASO comprises one or more 5′-methyl-cytosine nucleobases. In some aspects, the ASO is from 15 to 25 nucleotides in length, e.g., about 16, about 16, about 17, about 18, about 19, about 20, about 21, about 22, about 23, about 24, or about 25 nucleotides in length. In some aspects, the ASO comprises one or more modified internucleoside linkages. In some aspects, the one or more modified internucleoside linkages is a phosphorothioate linkage (PS). In some aspects, at least about 75%, at least about 80%, at least about 85%, at least about 90%, at least about 95%, or about 100% of internucleoside linkages are modified. In some aspects, each of the internucleoside linkages in the ASO is a phosphorothioate linkage.

### BRIEF DESCRIPTION OF FIGURES

FIGS. 1A-1G show that chromatin relaxation potentiates ASO1′s effect on E7 inclusion in cultures of the human embryonic kidney cell line HEK293. Alternative splicing was analyzed by radioactive RT-PCR followed by native polyacrylamide gel electrophoresis and autoradiography. Bars display means ± SD of percentage of the radioactivity in the FL band over the sum of radioactivity measurements in the FL and ΔE7 bands of at least three independent transfection experiments. A two-tailed Student’s t test was used to determine the significance between events. (FIG. 1A) HEK293T cells were co-transfected with the SMN2 minigene and expression vectors for WT^{res} and hC4 Pol II, followed by addition of α-amanitin. (FIG. 1B) Effect of camptothecin (CPT) on SMN2 E7 alternative splicing. HEK293T cells were incubated for at least 6 hours with 3 µM CPT. Combined effects on endogenous SMN2 E7 alternative splicing of transfection with 25 nM ASO1 and treatment with 3 µM TSA for 24 hr (FIG. 1C), 10 mM VPA for 24 hr (FIG. 1D), or 25 µM 5-AZA for 48 hr (FIG. 1E). Combined effects on HEK293T endogenous SMN2 E7 alternative splicing of transfection with 25 nM each of si-hnRNPA1 and si-hnRNPA2, and treatment with 3 µM TSA for 24 hr (FIG. 1F) or 10 mM VPA for 24 hr (FIG. 1G).

FIGS. 2A-2E show that transfection with ASO1 of HEK3293 cells promotes H3K9 dimethylation along the SMN2 gene, imposing an RNAPII roadblock (FIG. 2A) H3K9Ac distribution along the SMN2 gene, assessed by ChIP-qPCR in HEK293T cells treated (VPA) or untreated (CTRL.) with 10 mM VPA for 12 hr. H3K9me2 (FIG. 2B), total RNAPII (FIG. 2C), P-Ser5 RNAPII (FIG. 2D), and P-Ser2 RNAPII (FIG. 2E) distribution along the SMN2 gene, assessed by ChIP-qPCR in HEK293T cells transfected with the scramble oligo (CTRL) or ASO1 and treated with 10 mM VPA. Four independent immunoprecipitation replicates were conducted per experiment. Data are represented as mean ± S.D. (n = 4, *p < 0.05, two-tailed Student’s t test).

FIGS. 3A-3F show that transfection of HEK293 cells with another intronic ASO also promotes H3K9 dimethylation and imposes an RNAPII roadblock. FIG. 3A is a schematic representation of two opposing roles of ASO1. In the absence of HDAC inhibition, ASO1 promotes chromatin condensation (left); in the presence of HDAC inhibitors, chromatin becomes more relaxed, counteracting the condensation promoted by ASO1 (right). FIG. 3B shows the effects on endogenous SMN2 E7 alternative splicing when treating HEK293T cells with 3 µM CPT for 24 hr and transfection with 25 nM ASO2, separately or in combination. Total distribution of H3K9me2 (FIG. 3C), total RNAPII (FIG. 3D), P-Ser5 RNAPII (FIG. 3E), and P-Ser2 RNAPII (FIG. 3F) along the SMN2 gene is shown. This distribution was assessed by ChIP-qPCR in HEK293T cells transfected with the scramble oligo (CTRL) or ASO2 and treated with 10 mM VPA. Four independent immunoprecipitation replicates were conducted per experiment. Data are represented as mean ± S.D. (n = 4, *p < 0.05, two-tailed Student’s t test).

FIGS. 4A-4D show that ASO1 and VPA together promote a synergistic effect in SMA mice. Kaplan-Meier survival plot (FIG. 4A) and growth curves (FIG. 4B) of SMA mice, following subcutaneous administration at P0 and P1 of 16.8 µg ASO1 (n=18) or saline (n=14), one subcutaneous dose before P3 of 10 µg per g of body weight VPA (n=12), or both treatments together (n=20). ASO1-treated Smn^{+/-} heterozygote littermates (n=15) served as controls. Righting reflex (FIG. 4C) and grip strength (FIG. 4D) of P7 SMA animals, treated as indicated in FIG. 4A and FIG. 4B. Scramble (Ctrl) (n=12, 40 trials), VPA (n=13, 50 trials), ASO1 (n=11, 50 trials), ASO1 + VPA (n=17, 68 trials), and untreated heterozygotes (n=12, 36 trials). Statistical significance was analyzed by two-way repeated measures ANOVA. P < 0.05 was considered statistically significant; data are represented as mean + SD. In the righting-reflex test, the time it took a mouse to right itself on its back on a flat surface was measured. In the grip-strength test, the angle at which the pups fell from a tablet with a rough to which they held on by their forelimbs was measured.

FIGS. 5A-5F show that chromatin relaxation potentiates the ASO effect on E7 inclusion. Combined effects on endogenous SMN2 E7 alternative splicing in transfected (FIG. 5A) HEK293T cells with 25 nM ASO1 and increasing doses of TSA for 24 hr, (FIG. 5B) HeLa cells with 10 nM ASO1 and 3 µM TSA for 24 hr., and (FIG. 5C) SMA patient fibroblasts (3813) with 10 nM ASO1 and 3 µM TSA for 24 hr. Bars display means ± SD of percentage of the radioactivity in the FL band over the sum of radioactivity measurements in the FL and ΔE7 bands of at least three independent transfection experiments. (FIG. 5D and FIG. 5E) Transcriptional activation was assessed by RT-qPCR. (FIG. 5F) HEK293T cells with 3 nM ASO1 and 10 mM VPA for 24 hr. Bars indicate mean transcriptional activation ± SD ratios between precursors of SMN2 and GAPDH as the control gene. Statistical significance was analyzed by two-tailed Student’s t-tests. P < 0.05 was considered statistically significant.

FIGS. 6A-6D show antisense oligonucleotides and their effects on chromatin. FIG. 6A shows an excerpt of the SMN2 gene sequence (SEQ ID NO:57) comprising the E7 alternative exon. Uppercase, exonic nucleotide sequence. Lowercase, intronic sequences. Upstream highlight, binding site for ASO1. Downstream highlight, binding site for ASO2. Levels of H3K9me2 deposition along small EDRK-rich factor 1A (SERF1A, upstream of SMN2) (FIG. 6B), Myoblast Determination Protein (MYOD) (FIG. 6C), and Glyceraldehyde 3-phosphate dehydrogenase (GAPDH) (FIG. 6D), were assessed by ChIP-qPCR in HEK293T cells. Data represented as mean ± S.D. (n = 4, *p < 0.05, two-tailed Student’s t test).

FIGS. 7A-H show ASO1 and TSA combined treatment in mice. Kaplan-Meier survival plot (FIG. 7A) and growth curves (FIG. 7B) of SMA mice after subcutaneous administration at P0 and P1 of 16.8 µg ASO1 (n=20) or vehicle (n=12), one subcutaneous dose of 10 µg per g of body weight TSA (n=15) at P2, or both treatments together (n=24), are shown. ASO1-treated heterozygotes (n=18) served as controls. Statistical significance was analyzed by two-way repeated measures ANOVA. P < 0.05 was considered statistically significant; data are represented as mean + SD. Western-blot analysis of P7 tissues from Liver (FIG. 7C), kidney (FIG. 7D), muscle (FIG. 7E), spinal cord (FIG. 7F), brain (FIG. 7G) and heart (FIG. 7H) from SMA mice, using hSMN (BD Biosciences, upper panel) and β-tubulin (Sigma, lower panel) antibodies, are shown.

FIGS. 8A and 8B show that the slow mutant of RNAPII promotes E7 skipping in the endogenous SMN2 gene. FIG. 8A shows a comparison of the effects of the slow RNAPII mutant on the SMN2 alternative splicing reporter minigene (left) as in FIGS. 1A-1G, and on the endogenous SMN2 gene (right). FIG. 8B is a control experiment showing that the slow RNAPII mutant promotes higher inclusion of the class I exon EDI (also known as EDA or E33) of the human fibronectin (FN1) gene. Experimental conditions for assessing FN-EDI alternative splicing were previously described (de la Mata et al. Cell 12, 525-532, 2003). Alternative splicing was analyzed by radioactive RT-PCR followed by native polyacrylamide gel electrophoresis and autoradiography. Bars display means ± SD of percentage of the radioactivity in the FL band over the sum of radioactivity in the FL and D7 bands of at least three independent transfection experiments. A two-tailed Student’s t test was used to determine the significance between events

FIGS. 9A and 9B show that fast mutant of RNAPII promotes E7 inclusion in the endogenous SMN2 gene. The data shown correspond to a re-analysis of the RNA-seq data published by the Bentley laboratory (Fong et al. Genes Dev. 28, 2663-2676, 2014). Due to the almost identical DNA sequences of the human SMN1 and SMN2 genes (only 11 bp differences in 20 kbp), sequencing reads were assigned to a merge of the two genes. For this reason, splicing junction data had to be corrected considering 100% inclusion in the case of the SMN1 gene. FIG. 9A shows “Sashimi plots” indicating the number of reads for the E6-8 (skipping), E7-E8 (inclusion) and E6-E7 (inclusion) junctions of the SMN½ merged gene in cells stably transfected with the slow (R749H), fast (E1126G) and WT RNAPII large subunit constructs, all of which had a second mutation that confers resistance to α-amanitin, a drug that was used to treat the cells before RNA extraction. FIG. 9B shows raw and corrected quantification of the levels of E7 inclusion as assessed by analysis of the E6-E7 junction reads (top) and E7-E8 junction reads (bottom). Inclusion levels are expressed as both percentage of E7 inclusion and E7+/ E7- (i.e. FL/D7) ratios.

FIGS. 10A-10D show control experiments for the opposing roles of ASO1 on chromatin and splicing. FIG. 10A shows that overexpression of the SMN protein does not alter the ASO1 effect on E7 splicing. Top: control Western blot for efficient expression of the c-Myc-SMN fusion protein encoded by plasmid pcDNA3.1+SMN1mycHIS (Addgene, cat.# 71687) using anti-tubulin (tub.) as loading control. Bottom: SMN2 E7 RT-PCR of cells co-transfected with ASO1 and plasmid. FIG. 10B shows that transfection of HEK293T cells with ASO1 does not increase H3K27 trimethylation or H3K9 acetylation over the SMN2 gene. Distribution of these histone marks was assessed by ChIP-qPCR, with amplicons mapping near the numbered exons (E) and introns (i). Three independent immunoprecipitation replicates were conducted per experiment. Data are represented as mean ± S.D. (n = 5; in all cases p was much higher than 0.05, two-tailed Student’s t test). FIG. 10C shows that overexpression of the R-loop disrupting RNase H enzyme does not affect the ASO1 effect on E7 splicing. Top: control Western blot for efficient expression of the GFPRNaseH fusion protein encoded by plasmid pEGFP-RNASEH1 (Addgene, cat.# 108699), using anti-actin (act.) as loading control. Bottom: SMN2 E7 RT-PCR of cells co-transfected with ASO1 and the RNase H plasmid. FIG. 10D shows that siRNA-mediated AGO1 knockdown does not alter the ASO1 effect on E7 splicing. Top: control Western blot for efficient AGO1 knockdown using anti-tubulin (tub.) as loading control. Bottom: SMN2 E7 RT-PCR of cells co-transfected with ASO1 and the AGO1 siRNA. RT-PCR conditions for FIGS. 10A, 10C and 10D were as in FIGS. 1A-1G .

FIGS. 11A-11G show a genome-wide analysis of ASO1 effects on H3K9me2 mark deposition. Fold enrichments of ChIP-seq H3K9me2 reads of HEK293T cells upon different treatments (ASO1/control, ASO1 + VPA/control + VPA, and VPA vs. control), on the merged sequences of the SMN1 and SMN2 genes (FIG. 11A), on the human actin B gene (FIG. 11B) and on an approximately 40 kb region centered on the ASO1 target site (FIG. 11C) are shown. Frequency distributions of 11,738 protein-coding human genes with respect to the ASO1/control (FIG. 11D) and ASO1+VPA/control+VPA (FIG. 11E) fold increases in H3K9me2 reads are also shown. FIG. 11F and 11FG show a similar analysis for each of the 22 + X chromosomes. Y chromosome is absent because HEK293T cells were obtained from a female fetus. Expected and observed fold increase values are depicted by grey and red vertical dotted lines, respectively.

FIGS. 12A and 12B show that SMA mice injected with ASO1 present increased H3K9 dimethylation and RNAPII roadblocks along the SMN2 gene in brain and liver. H3K9me2 (left) and total RNAPII (right) distribution along the human SMN2 transgene, assessed by ChIP-qPCR, in brain (FIG. 12A) and liver (FIG. 12B) of P7 SMA mice injected with ASO1. Vertical arrows indicate the approximate location of the target site for ASO1 on the pre-mRNA. Three independent immunoprecipitation replicates were conducted per experiment. Data are represented as mean ± S.D. (n = 3, *p < 0.05, two-tailed Student’s t test).

### DETAILED DESCRIPTION

The present disclosure is directed to compositions and method to treat neuromuscular diseases, e.g., SMA, comprising administering a therapeutically effective amount of a composition to a subject in need thereof wherein the composition comprises (i) an antisense oligonucleotide (ASO) complementary to a nucleotide sequence within intron 7 of human SMN2 pre-mRNA; and (ii) a subclinical dose of a histone deacetylate inhibitor.

The present disclosure shows that SMN2 E7 is a class II exon, and that fast transcriptional elongation, caused by chromatin relaxation due to histone acetylation, promotes E7 inclusion. The disclosure shows that histone deacetylase (HDAC) inhibitors, such as valproic acid (VPA) or trichostatin A (TSA) act synergistically with nusinersen and nusinersen analogs, variants, or derivatives thereof, e.g., the antisense oligonucleotide ASO1, to promote SMN2 E7 inclusion.

ASO1 promotes the deployment of the silencing histone mark H3K9me2 along the SMN2 gene, creating a roadblock for RNAPII elongation around the target site of nusinersen and nusinersen analogs, variants, or derivatives thereof, in intron 7. This effect is reminiscent of a transcriptional gene-silencing mechanism observed with intronic siRNAs (Alló et al., Nat. Struct. Mol. Biol. 16, 717-724, 2009). Thus, the present disclosure shows that nusinersen and nusinersen analogs, variants, or derivatives thereof act at two levels, with opposite effects: at the pre-mRNA level, nusinersen and nusinersen analogs, variants, or derivatives thereof promote E7 inclusion by displacing the splicing repressors hnRNPA1 and A2, whereas at the co-transcriptional level, they favor E7 skipping by promoting H3K9me2 marks and slowing down RNAPII elongation.

Though the former effect is stronger, such that there is a net increase in E7 inclusion, the experimental data presented herein demonstrates that HDAC inhibition (e.g., by valproic acid, trichostatin A, or combinations thereof) counteracts the undesired chromatin effects of nusinersen or nusinersen analogs, resulting in significantly higher and unexpected E7 inclusion. Combined systemic administration of nusinersen and nusinersen analogs and HDAC inhibitors (e.g., valproic acid, trichostatin A, or combinations thereof, administered at subclinical doses) in an SMA model shows strong synergistic effects on SMN expression in peripheral and central tissues, and consequently on growth, survival, and neuromuscular function.

Accordingly, co-administration of HDAC inhibitors (e.g., valproic acid, trichostatin, or combinations thereof, e.g., at subclinical doses) can be used to increase the clinical efficacy of nusinersen and analogs thereof, as well as other splicing-modulatory drugs. The increase in efficacy allows the administration of reduced doses of nusinersen, nusinersen analogs, or other splicing-modulatory drugs, therefore reducing side effects resulting from their administration. Furthermore, the administration of nusinersen, nusinersen analogs, variants, or derivatives thereof, or other splicing-modulatory drugs in combination with HDAC inhibitors (e.g., valproic acid, trichostatin, or combinations thereof e.g., at subclinical doses) can be used to successfully treat, improve, or prevents SMA symptoms and sequelae in SMA patients.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “antisense oligonucleotide” (ASO) refers to an oligomer or polymer of nucleosides, such as naturally occurring nucleosides or modified forms thereof, that are covalently linked to each other through internucleotide linkages. The ASO useful for the disclosure includes at least one non-naturally occurring nucleoside. An ASO is at least partially complementary to a target nucleic acid, such that the ASO hybridizes to the target nucleic acid sequence.

The term “nucleic acids” or “nucleotides” is intended to encompass plural nucleic acids. In some aspects, the term “nucleic acids” or “nucleotides” refers to a target sequence, e.g., pre-mRNAs, mRNAs, or DNAs in vivo or in vitro. When the term refers to the nucleic acids or nucleotides in a target sequence, the nucleic acids or nucleotides can be naturally occurring sequences within a cell. In other aspects, “nucleic acids” or “nucleotides” refer to a sequence in the ASOs of the disclosure. When the term refers to a sequence in the ASOs, the nucleic acids or nucleotides can be non-naturally occurring, i.e., chemically synthesized, enzymatically produced, recombinantly produced, or any combination thereof. In some aspects, the nucleic acids or nucleotides in the ASOs are produced synthetically or recombinantly, but are not a naturally occurring sequence or a fragment thereof. In some aspects, the nucleic acids or nucleotides in the ASOs are not naturally occurring because they contain at least one nucleoside analog that is not naturally occurring in nature.

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogs” herein. Herein, a single nucleotide can be referred to as a monomer or unit. In certain aspects, the term “nucleotide analogs” refers to nucleotides having modified sugar moieties. Non-limiting examples of the nucleotides having modified sugar moieties (e.g., LNA) are disclosed elsewhere herein. In other aspects, the term “nucleotide analogs” refers to nucleotides having modified nucleobase moieties. The nucleotides having modified nucleobase moieties include, but are not limited to, 5-methyl-cytosine, isocytosine, pseudoisocytosine, 5-bromouracil, 5-propynyluracil, 6-aminopurine, 2-aminopurine, inosine, diaminopurine, and 2-chloro-6-aminopurine. In some aspects, the terms “nucleotide”, “unit” and “monomer” are used interchangeably. It will be recognized that when referring to a sequence of nucleotides or monomers, what is referred to is the sequence of bases, such as A, T, G, C or U, and analogs thereof.

The term “nucleoside” as used herein is used to refer to a glycoside comprising a sugar moiety and a base moiety, and can therefore be used when referring to the nucleotide units, which are covalently linked by the internucleotide linkages between the nucleotides of the ASO. In the field of biotechnology, the term “nucleotide” is often used to refer to a nucleic acid monomer or unit. In the context of an ASO, the term “nucleotide” can refer to the base alone, i.e., a nucleobase sequence comprising cytosine (DNA and RNA), guanine (DNA and RNA), adenine (DNA and RNA), thymine (DNA) and uracil (RNA), in which the presence of the sugar backbone and internucleotide linkages are implicit. Likewise, particularly in the case of oligonucleotides where one or more of the internucleotide linkage groups are modified, the term “nucleotide” can refer to a “nucleoside.” For example, the term “nucleotide” can be used, even when specifying the presence or nature of the linkages between the nucleosides.

The term “nucleotide length” as used herein means the total number of the nucleotides (monomers) in a given sequence. The term “nucleotide length” is therefore used herein interchangeably with “nucleotide number.”

As one of ordinary skill in the art would recognize, the 5′ terminal nucleotide of an oligonucleotide does not comprise a 5′ internucleotide linkage group, although it can comprise a 5′ terminal group.

The compounds described herein can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates. In some aspects, the asymmetric center can be an asymmetric carbon atom. The term “asymmetric carbon atom” means a carbon atom with four different substituents. According to the Cahn-Ingold-Prelog Convention, an asymmetric carbon atom can be of the “R” or “S” configuration.

As used herein, the term “bicyclic sugar” refers to a modified sugar moiety comprising a 4 to 7 membered ring comprising a bridge connecting two atoms of the 4 to 7 membered ring to form a second ring, resulting in a bicyclic structure. In some aspects, the bridge connects the C2′ and C4′ of the ribose sugar ring of a nucleoside (i.e., 2′-4′ bridge), as observed in LNA nucleosides.

The term “region” when used in the context of a nucleotide sequence refers to a section of that sequence. For example, the phrase “region within a nucleotide sequence” or “region within the complement of a nucleotide sequence” refers to a sequence shorter than the nucleotide sequence located within the particular nucleotide sequence or the complement of the nucleotides sequence, respectively. The term “sub-sequence” or “subsequence” can also refer to a region of a nucleotide sequence.

The term “expression” as used herein refers to a process by which a polynucleotide produces a gene product, for example, a RNA or a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide, which is translated from a transcript.

The terms “identical” or percent “identity” in the context of two or more nucleic acids refer to two or more sequences that are the same or have a specified percentage of nucleotides that are the same, when compared and aligned (introducing gaps, if necessary) for maximum correspondence, not considering any substitutions as part of the sequence identity. The percent identity can be measured using sequence comparison software or algorithms or by visual inspection. Various algorithms and software are known in the art that can be used to obtain alignments of nucleotide sequences.

One such non-limiting example of a sequence alignment algorithm is the algorithm described in Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain aspects, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, California) or Megalign (DNASTAR) are additional publicly available software programs that can be used to align sequences. In certain aspects, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative aspects, the GAP program in the GCG software package, which incorporates the algorithm of Needleman and Wunsch (J. Mol. Biol. (48):444-453 (1970)) can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain aspects, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. One skilled in the art can determine appropriate parameters for maximal alignment by particular alignment software. In certain aspects, the default parameters of the alignment software are used.

In certain aspects, the percentage identity “X” of a first nucleotide sequence to a second nucleotide sequence is calculated as 100 x (Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

Different regions within a single polynucleotide target sequence that align with a polynucleotide reference sequence can each have their own percent sequence identity. It is noted that the percent value of sequence identity is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

The term “complement” as used herein indicates a sequence that is complementary to a reference sequence. It is well known that complementarity is the base principle of DNA replication and transcription as it is a property shared between two DNA or RNA sequences, such that when they are aligned antiparallel to each other, the nucleotide bases at each position in the sequences will be complementary, much like looking in the mirror and seeing the reverse of things. Therefore, for example, the complement of a sequence of 5′ “ATGC” 3′ can be written as 3′ “TACG” 5′ or 5′ “GCAT” 3′. The terms “reverse complement”, “reverse complementary”, and “reverse complementarity” as used herein are interchangeable with the terms “complement”, “complementary”, and “complementarity.” In some aspects, the term “complementary” refers to 100% match or complementarity (i.e., fully complementary) to a contiguous nucleic acid sequence. In some aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a contiguous nucleic acid sequence.

The terms “corresponding to” and “corresponds to,” when referencing two separate nucleic acid or nucleotide sequences can be used to clarify regions of the sequences that correspond or are similar to each other based on homology and/or functionality, although the nucleotides of the specific sequences can be numbered differently. In addition, it is recognized that different numbering systems can be employed when characterizing a nucleic acid or nucleotide sequence. Further, it is recognized that the nucleic acid or nucleotide sequences of different variants of a nucleic acid can vary. As used herein, however, the regions of the variants that share nucleic acid or nucleotide sequence homology and/or functionality are deemed to “correspond” to one another.

The terms “corresponding nucleotide analog” and “corresponding nucleotide” are intended to indicate that the nucleobase in the nucleotide analog and the naturally occurring nucleotide have the same pairing, or hybridizing, ability. For example, when the 2-deoxyribose unit of the nucleotide is linked to an adenine, the “corresponding nucleotide analog” contains a pentose unit (different from 2-deoxyribose) linked to an adenine.

As used herein, the term “inhibiting,” e.g., the activity of a histone deacetylase refers to the reduction in the enzymatic activity of the histone deacetylase in a cell or a tissue. In some aspects, the term “inhibiting” refers to complete inhibition (100% inhibition or non-detectable level of deacetylase activity). In other aspects, the term “inhibiting” refers to at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95% or at least about 99% inhibition histone deacetylase activity in a cell or a tissue.

As used herein, the term “fragment” of a nucleic acid disclosed herein, e.g., an ASO, refers to a nucleic acid sequence that is shorter than a specific parent sequence, i.e., with 5′ and/or 3′ nucleotides deleted in comparison to parent sequence (a full length sequence as opposed to the fragment sequence). As used herein, the term “functional fragment” refers to a nucleic acid fragment (e.g., a shortened ASO) that retains the function of the parent sequence, at least partially. Accordingly, in some aspects, a functional fragment of an ASO disclosed herein retains the ability to bind to intron 7 of a pre-mRNA encoding human SMN2. Whether a fragment is a functional fragment can be assessed by any art known methods to determine whether the fragment can bind to the same target as the parent sequence, e.g., intron 7 of a pre-mRNA encoding human SMN2. In certain aspects, a functional fragment of an ASO disclosed herein retains at least about 50%, at least about 55%, at least about 60%, at least about 65%, at least about 70%, at least about 75%, at least about 80%, at least about 85%, at least about 90%, at least about 95%, or about 100% of the ability to bind to the same target as the parent sequence, e.g., intron 7 of a pre-mRNA encoding human SMN2.

As used herein, the term “variant” of a molecule refers to a molecule that shares certain structural and functional identities with another molecule upon comparison by a method known in the art. For example, a variant of a protein can include a substitution, insertion, deletion, frameshift or rearrangement in another protein.

In some aspects variants or variants of fragments of an ASO disclosed herein, e.g., nusinersen, share at least about 70%, at least about 80%, at least about 85%, at least about 90%, at least about 95%, at least about 96%, at least about 97%, at least about 98%, or at least about 99% sequence identity with the parent ASO, e.g., nusinersen.

Polynucleotide variants can contain alterations, e.g., substitutions, additions, or deletions that do not alter the properties or activities of the polynucleotide.

In some aspects, the variant or variant of a fragment of an ASO disclosed herein, e.g., nusinersen, retains the ability to be specifically targeted to intron 7 of a pre-mRNA encoding human SMN2. In some aspects, the variant includes one or more mutations.

As used herein, the term “nusinersen analog” refers to a nusinersen-like molecule with the same sequence as nusinersen and at least one nucleotide analog and/or a non-phosphodiester backbone linkage.

As used herein, the term “nusinersen variant” refers to a nusinersen-like molecule with the same sequence as nusinersen but having at least one mutation, e.g., a least one nucleobase substitution, insertion, or deletion.

As used herein, the term “nusinersen derivative” refers to a molecule comprising nusinersen or a nusinersen-like molecule and at least one heterologous moiety, e.g., a moiety that that increases plasma half-life, covalently attached to the nusinersen or nusinersen-like molecule.

As used herein, the term “nusinersen-like molecule” refers to a molecule, e.g., an antisense oligonucleotide, that can bind to the same mRNA target site as nusinersen and has at least one biological activity of nusinersen (e.g., promote SMN2 E7 inclusion). In some aspects, a nusinersen-like molecule disclosed herein is a nusinersen analog, a nusinersen variant, a nusinersen derivative, or a combination thereof.

The term “percent sequence identity” or “percent identity” between two polynucleotide sequences refers to the number of identical matched positions shared by the sequences over a comparison window, taking into account additions or deletions (i.e., gaps) that must be introduced for optimal alignment of the two sequences. A matched position is any position where an identical nucleotide is presented in both the target and reference sequence. Gaps presented in the target sequence are not counted since gaps are not nucleotides. Likewise, gaps presented in the reference sequence are not counted since target sequence nucleotides are counted, not nucleotides from the reference sequence.

The percentage of sequence identity is calculated by determining the number of positions at which the identical nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of nucleotide sequences. One suitable program to determine percent sequence identity is b12seq, part of the BLAST suite of programs available from the U.S. government’s National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs, and also available from the European Bioinformatics Institute (EBI) at www.ebi.ac.uk/Tools/psa.

Different regions within a single polynucleotide target sequence that aligns with a polynucleotide reference sequence can each have their own percent sequence identity. It is noted that the percent value of sequence identity is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of a molecule, e.g., an ASO, that have undergone one or more processes of purification. In some aspects, an isolated ASO composition has no detectable undesired activity or, alternatively, the level or amount of the undesired activity is at or below an acceptable level or amount. In other aspects, an isolated ASO composition has an amount and/or concentration of desired ASO at or above an acceptable amount and/or concentration. In other aspects, the isolated ASO composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated ASO preparations are substantially free of residual synthesis products. In some aspects, the isolated ASO preparations are 100% free, 99% free, 98% free, 97% free, 96% free, 95% free, 94% free, 93% free, 92% free, 91% free, or 90% free of any contaminants. Residual biological products can include abiotic materials (including chemicals) or unwanted nucleic acids. Substantially free of residual biological products can also mean that the ASO composition contains no detectable products and that only ASOs are detectable.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects, the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

“Administering,” as used herein, means to give a composition comprising a composition disclosed herein to a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. The compositions disclosed herein, e.g., nusinersen, nusinersen analogs, and other splicing-modulatory drugs in combination with histone deacetylase inhibitor (e.g., valproic acid, trichostatin A, or combination thereof) can be administered as part of a pharmaceutical composition comprising at least one excipient.

The terms “composition disclosed herein,” “composition of the present disclosure” and grammatical variants thereof (e.g., plural forms) refer to a combination treatment comprising (i) at least one splicing-modulatory drug, e.g., an ASO such as nusinersen, a nusinersen analog, a nusinersen variant, a nusinersen derivative, a non-ASO splicing-modulatory drug, or a combination thereof, and (ii) at least one histone deacetylase inhibitor, e.g., valproic acid, trichostatin A, or combination thereof. In some aspects, the term composition disclosed herein refers to nusinersen and valproic acid. In other aspects, the term composition disclosed herein refers to a nusinersen-like ASO, e.g., ASO1, and valproic acid. In other aspects, the term composition disclosed herein refers to (i) nusinersen a nusinersen variant, a nusinersen derivative, and (ii) trichostatin A. In some aspects, the term composition disclosed herein refers to (i) a nusinersen-like ASO, e.g., ASO1, and (ii) trichostatin A. The term also encompasses pharmaceutical compositions. The terms “method disclosed herein,” “method of the present disclosure” and grammatical variants thereof (e.g., plural forms) refer to methods, e.g., methods of treatment, practiced using compositions of the present disclosure.

An “effective amount” of, e.g., a composition disclosed herein, is an amount sufficient to carry out a specifically stated purpose, e.g., to treat one or more symptoms and/or sequelae of SMA. An “effective amount” can be determined empirically and in a routine manner, in relation to the stated purpose.

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also includes prophylaxis or prevention of a disease or condition or its symptoms thereof.

“Prevent” or “preventing,” as used herein, refers to decreasing or reducing the occurrence or severity of a particular outcome. In some aspects, preventing an outcome is achieved through prophylactic treatment. In some aspects, a composition disclosed herein is administered to a subject prophylactically.

### II. Compositions and Methods of Treatment

The present disclosure provides compositions and methods for the treatment of SMA. In some aspects, the compositions disclosed herein comprise an antisense oligonucleotide (ASO) complementary to a nucleotide sequence within intron 7 of human SMN2 pre-mRNA (e.g., an ASO of SEQ ID NO: 1, nusinersen, an analog thereof, a variant thereof, a derivative thereof, or a combination thereof) and a histone deacetylate inhibitor (e.g., valproic acid, trichostatin A, or a combination thereof; e.g., at a subclinical dose), wherein the histone deacetylate inhibitor acts synergically with the ASO resulting, e.g., in an increase in the survival time of SMA patients.

In some aspects, the histone deacetylase inhibitor (e.g., valproic acid, trichostatin A, or a combination thereof) is administered at a subclinical dose. As used herein, the term “subclinical dose” refers to a dose of histone deacetylase inhibitor (e.g., valproic acid, trichostatin A, or a combination thereof) which when administered to SMA patients as a monotherapy results in no significant increase in time of survival, increase in body mass, increase in muscle coordination, improvement in neuromuscular function, or any combination thereof).

The present disclosure provides methods for treating SMA comprising administering a therapeutically effective amount of a composition to the present disclosure to a subject in need thereof wherein the composition comprises, e.g., an antisense oligonucleotide (ASO) complementary to a nucleotide sequence within intron 7 of human SMN2 pre-mRNA (e.g., an ASO of SEQ ID NO: 1, nusinersen, an analog thereof, a variant thereof, a derivative thereof, or a combination thereof) and a histone deacetylate inhibitor (e.g., valproic acid, trichostatin A, or a combination thereof), and wherein the histone deacetylate inhibitor acts synergically with the ASO resulting, e.g., in an increase in survival time of the subject. In some aspects, the method comprises the administration of the histone deacetylase inhibitor at a subclinical dose (e.g., a dose at which no effect on the survival of SMA patients in observed),

The present disclosure also provides methods to increase the survival time of an SMA patient comprising administering a therapeutically effective amount of a composition to the present disclosure to the patient, wherein the composition comprises an antisense oligonucleotide (ASO) complementary to a nucleotide sequence within intron 7 of human SMN2 pre-mRNA (e.g., an ASO of SEQ ID NO: 1, nusinersen, an analog thereof, a variant thereof, a derivative thereof, or a combination thereof) and a histone deacetylate inhibitor (e.g., valproic acid, trichostatin A, or a combination thereof) and wherein the histone deacetylate inhibitor acts synergically with the ASO resulting, e.g., in an increase in the survival time of SMA patients. In some aspects, the method to increase survival time comprises administering the histone deacetylase inhibitor at a subclinical dose (e.g., a dose at which no effect on the survival of SMA is observed).

The use of a histone deacetylase inhibitor, e.g., valproic acid, trichostatin A, or a combination thereof, in combination with a splicing-modulatory ASO drug (e.g., an ASO complementary to a nucleotide sequence within intron 7 of human SMN2 pre-mRNA such as an ASO of SEQ ID NO: 1, nusinersen, an analog thereof, a variant thereof, a derivative thereof, or a combination thereof) allows a reduction of the dose of splicing-modulatory ASO drug administered to the patient, therefore reducing the incidence of side effects or adverse effects. Thus, the present disclosure also provides a method of reducing the incidence of side effects caused by a splicing-modulatory ASO drug (e.g., an ASO complementary to a nucleotide sequence within intron 7 of human SMN2 pre-mRNA such as nusinersen) comprising co-administering a histone deacetylate inhibitor (e.g., valproic acid, trichostatin A, or a combination thereof) to the subject receiving the splicing-modulatory ASO drug, wherein the histone deacetylate inhibitor acts synergically with the ASO, and wherein the dosage of splicing-modulatory ASO drug is lower than the recommended splicing-modulatory ASO drug dose administered in the absence of histone deacetylate inhibitor.

In some aspects, the method of reducing the incidence of side effects disclosed here comprises administering the histone deacetylase inhibitor at a subclinical dose (e.g., a dose at which no effect on the survival of SMA patients or other treatment milestones are observed, e.g., weight gain, increase in muscle, improvement in muscular coordination, improvement in neuromuscular function, or any combination thereof).

The present disclosure also provides a method to increase the clinical efficacy of a splicing-modulatory ASO drug (e.g., an ASO complementary to a nucleotide sequence within intron 7 of human SMN2 pre-mRNA such as nusinersen) comprising co-administering a histone deacetylase inhibitor, e.g., valproic acid, trichostatin A, or a combination thereof, wherein the increase in therapeutic efficacy (measured, e.g., as an increased in survival, weight-gain, or improvement in neuromuscular function, or a combination thereof) allows a reduction of the dose of splicing-modulatory ASO drug administered to the patient compared to the standard dose received by a patient no receiving the histone deacetylase inhibitor.

In some aspects, the present disclosure pertains to a method of increasing the level, expression and/or activity of exon 7-containing SMN2 mRNA or its gene product in a cell or organism comprising contacting the cell with or administering to the organism
(i) an oligonucleotide composition, e.g., an ASO, wherein the oligonucleotide composition comprises oligonucleotides of a base sequence which is at least about 70%, at least about 75%, at least about 80%, at least about 85%, at least about 90%, at least about 95%, at least about 96%, at least about 97%, at least about 98%, at least about 99%, or about 100% complementary to intron 7 of the SMN2 gene over the entire length of the oligonucleotide composition and at least 85% complementary to the sequence of CCAGCAUU (SEQ ID NO: 57), CCAGCAUUAUGAAAG (SEQ ID NO:81), CCAGCAUUAUGAAAGUGA (SEQ ID NO:82), CCAGCAUUAUGAAAGUGAAU (SEQ ID NO:83), or CCAGCNNNNNGAAAG (SEQ ID NO:84), wherein each T can be independently replaced by U and vice versa; and
(ii) a histone deacetylase inhibitor, e.g., valproic acid, trichostatin A, or a combination thereof (e.g., a subclinical dose);
wherein the level, expression and/or activity of exon 7-containing SMN2 mRNA or its gene production in the cell or organism is increased. In some aspects, the oligonucleotide composition, e.g., an ASO disclosed herein, and the histone deacetylase inhibitor act synergically. In some aspects, the histone deacetylase inhibitor acting synergically is administered at a concentration or dosage that has no effect on clinical manifestations of SMA and/or survival in the absence of treatment with an oligonucleotide composition, e.g., an ASO, disclosed herein (e.g., ASO1 or nusinersen, an analog thereof, a variant thereof, a derivative thereof, or a combination thereof), i.e., the histone deacetylase inhibitor (e.g., valproic acid, trichostatin A, or a combination thereof) is administered at a subclinical dose.

In some aspects, the oligonucleotide composition, e.g., an ASO disclosed herein (e.g., an ASO of SEQ ID NO: 1, nusinersen, an analog thereof, a variant thereof, a derivative thereof, or a combination thereof), is chirally controlled and/or further comprises an additional chemical moiety capable of binding to an asialoglycoprotein receptor (e.g., a GalNAc).

In some aspects, the present disclosure pertains to a method of increasing the level, expression and/or activity of exon 7-containing SMN2 mRNA or its gene product in a cell or organism comprising contacting the cell with or administering to the organism
(i) an oligonucleotide composition, e.g., an ASO, wherein the oligonucleotide composition comprises a sequence which is at least about 70%, at least about 75%, at least about 80%, at least about 85%, at least about 90%, at least about 95%, at least about 96%, at least about 97%, at least about 98%, at least about 99%, or about 100% identical to ATTCACTTTCATAATGCTGG (ASO1) (SEQ ID NO: 1), TGCTGGCAGACTTAC (SEQ ID NO:58), CATAATGCTGGCAGA (SEQ ID NO:59), TCATAATGCTGGCAG (SEQ ID NO:60), TTCATAATGCTGGCA (SEQ ID NO:61), TTTCATAATGCTGGC (SEQ ID NO:62), TCACTTTCATAATGCTGG (nusinersen) (SEQ ID NO:63), AGTAAGATTCACTTT (SEQ ID NO:64), CTTTCATAATGCTGG (SEQ ID NO:65), TCATAATGCTGG (SEQ ID NO:66), ACTTTCATAATGCTG (SEQ ID NO:67), TTCATAATGCTG (SEQ ID NO:68), CACTTTCATAATGCT (SEQ ID NO:69), TTTCATAATGCT (SEQ ID NO:70), TCACTTTCATAATGC (SEQ ID NO:71), CTTTCATAATGC (SEQ ID NO:72), TTCACTTTCATAATG (SEQ ID NO:73), ACTTTCATAATG (SEQ ID NO:74), ATTCACTTTCATAAT (SEQ ID NO:75), CACTTTCATAAT (SEQ ID NO:76), GATTCACTTTCATAA (SEQ ID NO:77), TCACTTTCATAA (SEQ ID NO:78), TTCACTTTCATA (SEQ ID NO:79), or ATTCACTTTCAT (SEQ ID NO:80); and
(ii) a histone deacetylase inhibitor, e.g., valproic acid, trichostatin A, or a combination thereof, e.g., at a subclinical dose;
wherein the level, expression and/or activity of exon 7-containing SMN2 mRNA or its gene production the cell is increased. In some aspects, the oligonucleotide composition, e.g., an ASO disclosed herein (e.g., an ASO of SEQ ID NO: 1, nusinersen, an analog thereof, a variant thereof, a derivative thereof, or a combination thereof), and the histone deacetylase inhibitor (e.g., valproic acid, trichostatin A, or a combination thereof; e.g., at a subclinical dose) act synergically. In some aspects, the histone deacetylase inhibitor (e.g., valproic acid, trichostatin A, or a combination thereof; e.g., at a subclinical dose) is administered at a concentration or dosage that has no effect of clinical manifestations of SMA and/or survival in the absence of an oligonucleotide composition disclosed herein, e.g., nusinersen.

In some aspects, the histone deacetylase inhibitors disclosed herein, e.g., valproic acid and trichostatin A, show a lack of a therapeutic effect per se when administered to SMA patients. However, the histone deacetylase inhibitors disclosed herein, e.g., valproic acid and trichostatin, show a strong effect in improving the therapeutic effects of oligonucleotide compositions, e.g., ASOs such as nusinersen, targeting exon 7-containing SMN2 mRNA.

The present disclosure provides dose amounts and frequencies for the components of the compositions and methods of treatment of the present disclosure, i.e., (i) a splicing-modulatory drug (e.g., an ASO complementary to intron 7 of a nucleic acid encoding human SMN2 pre-mRNA such as nusinersen), and (ii) a histone deacetylase inhibitor, e.g., valproic acid, trichostatin A, or an combination thereof. In some aspects, the histone deacetylase inhibitor or combination thereof is administered at a concentration such that no significant therapeutic effect per se is observed in SMA patients and/or SMA model systems (e.g., a mouse model), i.e., it is administered at a subclinical dose.

In some aspects, at least one component of a composition of the present disclosure, e.g., the ASO or the histone deacetylase inhibitor is administered as a bolus injection. In some aspects, the dose of the bolus injection is from about 0.01 to about 25 milligrams of ASO (e.g., nusinersen) per kilogram body weight of the subject. In some aspects, the dose of the bolus injection is from about 0.01 to about 10 milligrams of ASO (e.g., nusinersen) per kilogram body weight of the subject. In some aspects, the dose of ASO is from about 0.05 to about 5 milligrams of ASO (e.g., nusinersen) per kilogram body weight of the subject. In some aspects, the dose is from about 0.1 to about 2 milligrams of ASO (e.g., nusinersen) per kilogram body weight of the subject. In some aspects, the dose is from about 0.5 to about 1 milligrams of ASO (e.g., nusinersen) per kilogram body weight of the subject.

In some aspects, the ASO (e.g., ASO1 of SEQ ID NO: 1, nusinersen, an analog thereof, a variant thereof, a derivative thereof, any ASO targeting SMN2 mRNA disclosed in the present specification, or a combination thereof) can be administered at a dosage of about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.5 mg/kg, about 3 mg/mg, about 3.5 mg/kg, about 4 mg/kg, about 4.5 mg/kg, about 5 mg/kg, about 5.5 mg/kg, about 6 mg/kg, about 6.5 mg.kg, about 7 mg/kg, about 7.5 mg/kg, about 8 mg/kg, about 8.5 mg/kg, about 9 mg/kg, about 9.5 mg/kg, about 10 mg/kg, about 11 mg/kg, about 12 mg/kg, about 13 mg/kg, about 14/mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, or about 25 mg/kg per dose.

In some aspects, the ASO (e.g., ASO1 of SEQ ID NO: 1, nusinersen, an analog thereof, a variant thereof, a derivative thereof, any ASO targeting SMN2 mRNA disclosed in the present specification, or a combination thereof) can be administered at a dosage of at least 0.01 mg/kg, at least 0.02 mg/kg, at least 0.03 mg/kg, at least 0.04 mg/kg, at least 0.05 mg/kg, at least 0.06 mg/kg, at least 0.07 mg/kg, at least 0.08 mg/kg, at least 0.09 mg/kg, at least 0.1 mg/kg, at least 0.2 mg/kg, at least 0.3 mg/kg, at least 0.4 mg/kg, at least 0.5 mg/kg, at least 0.6 mg/kg, at least 0.7 mg/kg, at least 0.8 mg/kg, at least 0.9 mg/kg, at least 1 mg/kg, at least 1.1 mg/kg, at least 1.2 mg/kg, at least 1.3 mg/kg, at least 1.4 mg/kg, at least 1.5 mg/kg, at least 1.6 mg/kg, at least 1.7 mg/kg, at least 1.8 mg/kg, at least 1.9 mg/kg, at least 2 mg/kg, at least 2.5 mg/kg, at least 3 mg/mg, at least 3.5 mg/kg, at least 4 mg/kg, at least 4.5 mg/kg, at least 5 mg/kg, at least 5.5 mg/kg, at least 6 mg/kg, at least 6.5 mg.kg, at least 7 mg/kg, at least 7.5 mg/kg, at least 8 mg/kg, at least 8.5 mg/kg, at least 9 mg/kg, at least 9.5 mg/kg, at least 10 mg/kg, at least 11 mg/kg, at least 12 mg/kg, at least 13 mg/kg, at least 14/mg/kg, at least 15 mg/kg, at least 16 mg/kg, at least 17 mg/kg, at least 18 mg/kg, at least 19 mg/kg, at least 20 mg/kg, at least 21 mg/kg, at least 22 mg/kg, at least 23 mg/kg, at least 24 mg/kg, or at least 25 mg/kg per dose.

In some aspects, the ASO (e.g., ASO1 of SEQ ID NO:1, nusinersen, an analog thereof, a variant thereof, a derivative thereof, any ASO targeting SMN2 mRNA disclosed in the present specification, or a combination thereof) can be administered at a dosage between about 0.01 mg/kg and about 0.05 mg/kg, between about 0.05 mg/kg and about 0.1 mg/kg, between about 0.1 mg/kg and about 0.5 mg/kg, between about 0.5 mg/kg and about 1 mg/kg, between about 1 mg/kg and about 2 mg/kg, between about 2 mg/kg and about 3 mg/kg, between about 3 mg/kg and about 4 mg/kg, between about 4 mg/kg and about 5 mg/kg, between about 5 mg/kg and about 6 mg/kg, between about 6 mg/kg and about 7 mg/kg, between about 7 mg/kg and about 8 mg/kg, between about 8 mg/kg and about 9 mg/kg, between about 9 mg/kg and about 10 mg/kg, between about 10 mg/kg and about 11 mg/kg, between about 11 mg/kg and about 12 mg/kg, between about 12 mg/kg and about 13 mg/kg, between about 13 mg/kg and about 14 mg/kg, between about 14 mg/kg and about 15 mg/kg, between about 15 mg/kg and about 16 mg/kg, between about 16 mg/kg and about 17 mg/kg, between about 17 mg/kg and about 18 mg/kg, between about 18 mg/kg and about 19 mg/kg, between about 19 mg/kg and about 20 mg/kg, between about 20 mg/kg and about 21 mg/kg, between about 21 mg/kg and about 22 mg/kg, between about 23 mg/kg and about 24 mg/kg, between about 24 mg/kg and about 25 mg/kg, between about 1 mg/kg and about 5 mg/kg, between about 5 mg/kg and about 10 mg/kg, between about 10 mg/kg and about 15 mg/kg, between about 15 mg/kg and about 20 mg/kg, between about 20 mg/kg and about 25 mg/kg per dose.

In some aspects, the ASO (e.g., ASO1 of SEQ ID NO:1, nusinersen, an analog thereof, a variant thereof, a derivative thereof, any ASO targeting SMN2 mRNA disclosed in the present specification, or a combination thereof) can be administered at a dosage lower that about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2 mg/kg, about 2.5 mg/kg, about 3 mg/mg, about 3.5 mg/kg, about 4 mg/kg, about 4.5 mg/kg, about 5 mg/kg, about 5.5 mg/kg, about 6 mg/kg, about 6.5 mg.kg, about 7 mg/kg, about 7.5 mg/kg, about 8 mg/kg, about 8.5 mg/kg, about 9 mg/kg, about 9.5 mg/kg, about 10 mg/kg, about 11 mg/kg, about 12 mg/kg, about 13 mg/kg, about 14/mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, or about 25 mg/kg per dose.

In some aspects, the ASO (e.g., ASO1 of SEQ ID NO:1, nusinersen, an analog thereof, a variant thereof, a derivative thereof, any ASO targeting SMN2 mRNA disclosed in the present specification, or a combination thereof) can be administered at a dosage of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.5 mg, about 3 mg/mg, about 3.5 mg, about 4 mg, about 4.5 mg, about 5 mg, about 5.5 mg, about 6 mg, about 6.5 mg.kg, about 7 mg, about 7.5 mg, about 8 mg, about 8.5 mg, about 9 mg, about 9.5 mg, about 10 mg, about 11 mg, about 12 mg, about 13 mg, about 14/mg, about 15 mg, about 16 mg, about 17 mg, about 18 mg, about 19 mg, about 20 mg, about 21 mg, about 22 mg, about 23 mg, about 24 mg, or about 25 mg per dose.

In some aspects, the ASO (e.g., ASO1 of SEQ ID NO:1, nusinersen, an analog thereof, a variant thereof, a derivative thereof, any ASO targeting SMN2 mRNA disclosed in the present specification, or a combination thereof) can be administered at a dosage of at least 0.01 mg, at least 0.02 mg, at least 0.03 mg, at least 0.04 mg, at least 0.05 mg, at least 0.06 mg, at least 0.07 mg, at least 0.08 mg, at least 0.09 mg, at least 0.1 mg, at least 0.2 mg, at least 0.3 mg, at least 0.4 mg, at least 0.5 mg, at least 0.6 mg, at least 0.7 mg, at least 0.8 mg, at least 0.9 mg, at least 1 mg, at least 1.1 mg, at least 1.2 mg, at least 1.3 mg, at least 1.4 mg, at least 1.5 mg, at least 1.6 mg, at least 1.7 mg, at least 1.8 mg, at least 1.9 mg, at least 2 mg, at least 2.5 mg, at least 3 mg/mg, at least 3.5 mg, at least 4 mg, at least 4.5 mg, at least 5 mg, at least 5.5 mg, at least 6 mg, at least 6.5 mg.kg, at least 7 mg, at least 7.5 mg, at least 8 mg, at least 8.5 mg, at least 9 mg, at least 9.5 mg, at least 10 mg, at least 11 mg, at least 12 mg, at least 13 mg, at least 14/mg, at least 15 mg, at least 16 mg, at least 17 mg, at least 18 mg, at least 19 mg, at least 20 mg, at least 21 mg, at least 22 mg, at least 23 mg, at least 24 mg, or at least 25 mg per dose.

In some aspects, the ASO (e.g., ASO1 of SEQ ID NO:1, nusinersen, an analog thereof, a variant thereof, a derivative thereof, any ASO targeting SMN2 mRNA disclosed in the present specification, or a combination thereof) can be administered at a dosage between about 0.01 mg and about 0.05 mg, between about 0.05 mg and about 0.1 mg, between about 0.1 mg and about 0.5 mg, between about 0.5 mg and about 1 mg, between about 1 mg and about 2 mg, between about 2 mg and about 3 mg, between about 3 mg and about 4 mg, between about 4 mg and about 5 mg, between about 5 mg and about 6 mg, between about 6 mg and about 7 mg, between about 7 mg and about 8 mg, between about 8 mg and about 9 mg, between about 9 mg and about 10 mg, between about 10 mg and about 11 mg, between about 11 mg and about 12 mg, between about 12 mg and about 13 mg, between about 13 mg and about 14 mg, between about 14 mg and about 15 mg, between about 15 mg and about 16 mg, between about 16 mg and about 17 mg, between about 17 mg and about 18 mg, between about 18 mg and about 19 mg, between about 19 mg and about 20 mg, between about 20 mg and about 21 mg, between about 21 mg and about 22 mg, between about 23 mg and about 24 mg, between about 24 mg and about 25 mg, between about 1 mg and about 5 mg, between about 5 mg and about 10 mg, between about 10 mg and about 15 mg, between about 15 mg and about 20 mg, between about 20 mg and about 25 mg per dose.

In some aspects, the ASO (e.g., ASO1 of SEQ ID NO:1, nusinersen, an analog thereof, a variant thereof, a derivative thereof, any ASO targeting SMN2 mRNA disclosed in the present specification, or a combination thereof) can be administered at a dosage lower that about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.5 mg, about 3 mg/mg, about 3.5 mg, about 4 mg, about 4.5 mg, about 5 mg, about 5.5 mg, about 6 mg, about 6.5 mg.kg, about 7 mg, about 7.5 mg, about 8 mg, about 8.5 mg, about 9 mg, about 9.5 mg, about 10 mg, about 11 mg, about 12 mg, about 13 mg, about 14/mg, about 15 mg, about 16 mg, about 17 mg, about 18 mg, about 19 mg, about 20 mg, about 21 mg, about 22 mg, about 23 mg, about 24 mg, or about 25 mg per dose.

Nusinersen is generally administered at 0.2 mg/kg. Accordingly, since the co-administration of a histone deacetylase inhibitor disclosed herein such as valproic acid potentiates the effect of the ASO, in some aspects, the ASO (e.g., ASO1 or nusinersen, an analog thereof, a variant thereof, a derivative thereof, or a combination thereof) can be administered at a dose lower than 0.2 mg/kg. For example, in some aspects, the ASO (e.g., ASO1 or nusinersen, an analog thereof, a variant thereof, a derivative thereof, or a combination thereof) can be administered at a dose lower than about 0.2 mg/kg, lower than about 0.19 mg/kg, lower than about 0.18 mg/kg, lower than about 0.17 mg/kg, lower than about 0.16 mg/kg, lower than about 0.15 mg/kg, lower than about 0.14 mg/kg, lower than about 0.13 mg/kg, lower than about 0.12 mg/kg, lower than about 0.11 mg/kg, lower than about 0.1 mg/kg, lower than about 0.09 mg/kg, lower than about 0.08 mg/kg, lower than about 0.07 mg/kg, lower than about 0.06 mg/kg, lower than about 0.05 mg/kg, lower than about 0.04 mg/kg, lower than about 0.03 mg/kg, lower than about 0.02 mg/kg, or lower than about 0.01 mg/kg per dose.

Nusinersen is generally administered at 12 mg/dose. Accordingly, since the co-administration of a histone deacetylase inhibitor disclosed herein such as valproic acid potentiates the effect of the ASO, in some aspects, the ASO (e.g., ASO1 or nusinersen, an analog thereof, a variant thereof, a derivative thereof, or a combination thereof) can be administered at a dose lower than 12 mg/dose. For example, in some aspects, the ASO (e.g., ASO1 or nusinersen, an analog thereof, a variant thereof, a derivative thereof, or a combination thereof) can be administered at a dose lower than about 12 mg/dose, lower than about 11 mg/dose, lower than about 10 mg/dose, lower than about 9 mg/dose, lower than about 8 mg/dose, lower than about 7 mg/dose, lower than about 6 mg/dose, lower than about 5 mg/dose, lower than about 4 mg/dose, lower than about 3 mg/dose, lower than about 2 mg/dose, or lower than about 1 mg/dose.

Nusinersen is generally administered at 12 mg/dose/day. Accordingly, since the co-administration of a histone deacetylase inhibitor disclosed herein such as valproic acid potentiates the effect of the ASO, in some aspects, the ASO (e.g., ASO1 or nusinersen, an analog thereof, a variant thereof, a derivative thereof, or a combination thereof) can be administered at a dose lower than 12 mg/dose/day. For example, in some aspects, the ASO (e.g., ASO1 or nusinersen, an analog thereof, a variant thereof, a derivative thereof, or a combination thereof) can be administered at a dose lower than about 12 mg/dose/day, lower than about 11 mg/dose/day, lower than about 10 mg/dose/day, lower than about 9 mg/dose/day, lower than about 8 mg/dose/day, lower than about 7 mg/dose/day, lower than about 6 mg/dose/day, lower than about 5 mg/dose/day, lower than about 4 mg/dose/day, lower than about 3 mg/dose/day, lower than about 2 mg/dose/day, or lower than about 1 mg/dose/day.

In some aspects, the ASO (e.g., ASO1, nusinersen, an analog thereof, a variant thereof, a derivative thereof, any ASO targeting SMN2 mRNA disclosed in the present specification, or a combination thereof) can be administered (before, after, or concurrently) with a histone deacetylase (e.g., valproic acid, trichostatin A, or a combination thereof, e.g., in a subclinical dose).

In some aspects, the histone deacetylase inhibitor (e.g., valproic acid, trichostatin A, or a combination thereof) can be administered at a dosage of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/mg, about 17 mg/kg, about 18 mg/kg, about 19 mg/kg, about 20 mg/kg, about 21 mg/kg, about 22 mg/kg, about 23 mg.kg, about 24 mg/kg, about 25 mg/kg, about 26 mg/kg, about 28 mg/kg, about 30 mg/kg, about 32 mg/kg, about 34 mg/kg, about 36 mg/kg, about 38 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg per dose.

In some aspects, the histone deacetylase inhibitor (e.g., valproic acid, trichostatin A, or a combination thereof) can be administered at a dosage of at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 11 mg/kg, at least 12 mg/kg, at least 13 mg/kg, at least 14 mg/kg, at least 15 mg/kg, at least 16 mg/mg, at least 17 mg/kg, at least 18 mg/kg, at least 19 mg/kg, at least 20 mg/kg, at least 21 mg/kg, at least 22 mg/kg, at least 23 mg.kg, at least 24 mg/kg, at least 25 mg/kg, at least 26 mg/kg, at least 28 mg/kg, at least 30 mg/kg, at least 32 mg/kg, at least 34 mg/kg, at least 36 mg/kg, at least 38 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg per dose.

In some aspects, the histone deacetylase inhibitor (e.g., valproic acid, trichostatin A, or a combination thereof) can be administered at a dosage between about 1 mg/kg and about 5 mg/kg, between about 5 mg/kg and about 10 mg/kg, between about 10 mg/kg and about 20 mg/kg, between about 20 mg/kg and about 30 mg/kg, between about 30 mg/kg and about 40 mg/kg, between about 40 mg/kg and about 50 mg/kg, between about 50 mg/kg and about 60 mg/kg, between about 60 mg/kg and about 70 mg/kg, between about 70 mg/kg and about 80 mg/kg, between about 80 mg/kg and about 90 mg/kg, between about 90 mg/kg and about 100 mg/kg, between about 15 mg/kg and about 25 mg/kg, between about 25 mg/kg and about 35 mg/kg, between about 35 mg/kg and about 45 mg/kg, between about 45 mg/kg and about 55 mg/kg, between about 55 mg/kg and about 65 mg/kg, between about 65 mg/kg and about 75 mg/kg, between about 75 mg/kg and about 85 mg/kg, between about 85 mg/kg and about 95 mg/kg, between about 10 mg/kg and about 30 mg/kg, between about 20 mg/kg and about 40 mg/kg, between about 30 mg/kg and about 50 mg/kg, between about 40 mg/kg and about 60 mg/kg, between about 50 mg/kg and about 70 mg/kg, between about 60 mg/kg and about 80 mg/kg, between about 70 mg/kg and about 90 mg/kg, between about 10 mg/kg and about 40 mg/kg, between about 20 mg/kg and about 50 mg/kg, between about 30 mg/kg and about 60 mg/kg, between about 40 mg/kg and about 70 mg/kg, between about 50 mg/kg and about 80 mg/kg, between about 60 mg/kg and about 90 mg/kg per dose.

In some aspects, the histone deacetylase inhibitor (e.g., valproic acid, trichostatin A, or a combination thereof) can be administered at a dosage lower than about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/mg, about 17 mg/kg, about 18 mg/kg, about 19 mg/kg, about 20 mg/kg, about 21 mg/kg, about 22 mg/kg, about 23 mg.kg, about 24 mg/kg, about 25 mg/kg, about 26 mg/kg, about 28 mg/kg, about 30 mg/kg, about 32 mg/kg, about 34 mg/kg, about 36 mg/kg, about 38 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg per dose.

In some aspects, the histone deacetylase inhibitor (e.g., valproic acid, trichostatin A, or a combination thereof) can be administered at a dosage of about 10 mg, about 20 mg, about 30 mg, about 40 mg, about 50 mg, about 60 mg, about 70 mg, about 80 mg, about 90 mg, about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg/mg, about 2300 mg, about 2400 mg, about 2500 mg, about 2600 mg, about 2700 mg, about 2800 mg, about 2900 mg.kg, about 3000 mg, about 3100 mg, about 3200 mg, about 3300 mg, about 3400 mg, about 3500 mg, about 3600 mg, about 3700 mg, about 3800 mg, about 3900 mg, or about 4000 mg.

In some aspects, the histone deacetylase inhibitor (e.g., valproic acid, trichostatin A, or a combination thereof) can be administered at a dosage of at least 10 mg, at least 20 mg, at least 30 mg, at least 40 mg, at least 50 mg, at least 60 mg, at least 70 mg, at least 80 mg, at least 90 mg, at least 100 mg, at least 200 mg, at least 300 mg, at least 400 mg, at least 500 mg, at least 600 mg, at least 700 mg, at least 800 mg, at least 900 mg, at least 1000 mg, at least 1100 mg, at least 1200 mg, at least 1300 mg, at least 1400 mg, at least 1500 mg, at least 1600 mg, at least 1700 mg, at least 1800 mg, at least 1900 mg, at least 2000 mg, at least 2100 mg, at least 2200 mg/mg, at least 2300 mg, at least 2400 mg, at least 2500 mg, at least 2600 mg, at least 2700 mg, at least 2800 mg, at least 2900 mg.kg, at least 3000 mg, at least 3100 mg, at least 3200 mg, at least 3300 mg, at least 3400 mg, at least 3500 mg, at least 3600 mg, at least 3700 mg, at least 3800 mg, at least 3900 mg, or at least 4000 mg.

In some aspects, the histone deacetylase inhibitor (e.g., valproic acid, trichostatin A, or a combination thereof) can be administered at a dosage between about 10 mg and about 20 mg, between about 20 mg and about 50 mg, between about 50 mg and about 100 mg, between about 100 mg and about 200 mg, between about 200 mg and about 300 mg, between about 300 mg and about 400 mg, between about 400 mg and about 500 mg, between about 500 mg and about 600 mg, between about 600 mg and about 700 mg, between about 700 mg and about 800 mg, between about 800 mg and about 900 mg, between about 900 mg and about 1000 mg, between about 1000 mg and about 1200 mg, between about 1200 mg and about 1400 mg, between about 1400 mg and about 1600 mg, between about 1600 mg and about 1800 mg, between about 1800 mg and about 2000 mg, between about 2000 mg and about 2200 mg, between about 2200 mg and about 2400 mg, between about 2400 mg and about 2600 mg, between about 2600 mg and about 2800 mg, between about 2800 mg and about 3000 mg, between about 3000 mg and about 3200 mg, between about 3200 mg and about 3400 mg, between about 3400 mg and about 3600 mg, between about 3600 mg and about 3800 mg, or between about 3800 mg and about 4000 per dose.

In some aspects, the histone deacetylase inhibitor (e.g., valproic acid, trichostatin A, or a combination thereof) can be administered at a dosage lower that about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg/mg, about 1200 mg, about 1300 mg, about 1400 mg, about 1500 mg, about 1600 mg, about 1700 mg, about 1800 mg.kg, about 1900 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, about 3600 mg, about 3700 mg, about 3800 mg, about 3900 mg, or about 4000 mg per dose. In some aspects, doses larger than, e.g., about 250 mg per dose, are administered as divided doses.

In some aspects, the histone deacetylase inhibitor (e.g., valproic acid, trichostatin A, or a combination thereof) can be administered at a dose lower than about 15 mg/kg, lower than about 15 mg/kg, lower than about 13 mg/kg, lower than about 12 mg/kg, lower than about 11 mg/kg, lower than about 10 mg/kg, lower than about 9 mg/kg, lower than about 8 mg/kg, lower than about 7 mg/kg, lower than about 6 mg/kg, lower than about 5 mg/kg, lower than about 4 mg/kg, lower than about 3 mg/kg, lower than about 2 mg/kg, lower than about 1 mg/kg per dose.

In some aspects, the histone deacetylase inhibitor (e.g., valproic acid, trichostatin A, or a combination thereof) can be administered at a dose lower than about 600 mg/dose, lower than about 550 mg/dose, lower than about 500 mg/dose, lower than about 550 mg/dose, lower than about 500 mg/dose, lower than about 450 mg/dose, lower than about 400 mg/dose, lower than about 350 mg/dose, lower than about 300 mg/dose, lower than about 250 mg/dose, lower than about 200 mg/dose, lower than about 175 mg/dose, lower than about 150 mg/dose, lower than about 125 mg/dose, lower than about 100 mg/dose, lower than about 90 mg/dose, lower than about 80 mg/dose, lower than about 70 mg/dose, lower than about 60 mg/dose, lower than about 50 mg/dose, lower than about 40 mg/dose, lower than about 30 mg/dose, lower than about 20 mg/dose, or lower than about 10 mg/dose.

In some aspects, the histone deacetylase inhibitor (e.g., valproic acid, trichostatin A, or a combination thereof) can be administered at a dose lower than about 600 mg/dose/day, lower than about 550 mg/dose/day, lower than about 500 mg/dose/day, lower than about 550 mg/dose/day, lower than about 500 mg/dose/day, lower than about 450 mg/dose/day, lower than about 400 mg/dose/day, lower than about 350 mg/dose/day, lower than about 300 mg/dose/day, lower than about 250 mg/dose/day, lower than about 200 mg/dose/day, lower than about 175 mg/dose/day, lower than about 150 mg/dose/day, lower than about 125 mg/dose/day, lower than about 100 mg/dose/day, lower than about 90 mg/dose/day, lower than about 80 mg/dose/day, lower than about 70 mg/dose/day, lower than about 60 mg/dose/day, lower than about 50 mg/dose/day, lower than about 40 mg/dose/day, lower than about 30 mg/dose/day, lower than about 20 mg/dose/day, or lower than about 10 mg/dose/day.

In some aspects, the ASO (e.g., ASO1 or nusinersen, an analog thereof, a variant thereof, a derivative thereof, or a combination thereof) can be administered at a dose lower than about 0.2 mg/kg, lower than about 0.19 mg/kg, lower than about 0.18 mg/kg, lower than about 0.17 mg/kg, lower than about 0.16 mg/kg, lower than about 0.15 mg/kg, lower than about 0.14 mg/kg, lower than about 0.13 mg/kg, lower than about 0.12 mg/kg, lower than about 0.11 mg/kg, lower than about 0.1 mg/kg, lower than about 0.09 mg/kg, lower than about 0.08 mg/kg, lower than about 0.07 mg/kg, lower than about 0.06 mg/kg, lower than about 0.05 mg/kg, lower than about 0.04 mg/kg, lower than about 0.03 mg/kg, lower than about 0.02 mg/kg, or lower than about 0.01 mg/kg per dose; and the histone deacetylase inhibitor (e.g., valproic acid, trichostatin A, or a combination thereof) can be administered at a dose lower than about 15 mg/kg, lower than about 15 mg/kg, lower than about 13 mg/kg, lower than about 12 mg/kg, lower than about 11 mg/kg, lower than about 10 mg/kg, lower than about 9 mg/kg, lower than about 8 mg/kg, lower than about 7 mg/kg, lower than about 6 mg/kg, lower than about 5 mg/kg, lower than about 4 mg/kg, lower than about 3 mg/kg, lower than about 2 mg/kg, lower than about 1 mg/kg per dose.

In some aspects, the ASO (e.g., ASO1 or nusinersen, an analog thereof, a variant thereof, a derivative thereof, or a combination thereof) can be administered at a dose lower than about 12 mg/dose, lower than about 11 mg/dose, lower than about 10 mg/dose, lower than about 9 mg/dose, lower than about 8 mg/dose, lower than about 7 mg/dose, lower than about 6 mg/dose, lower than about 5 mg/dose, lower than about 4 mg/dose, lower than about 3 mg/dose, lower than about 2 mg/dose, or lower than about 1 mg/dose; and the histone deacetylase inhibitor (e.g., valproic acid, trichostatin A, or a combination thereof) can be administered at a dose lower than about 600 mg/dose, lower than about 550 mg/dose, lower than about 500 mg/dose, lower than about 550 mg/dose, lower than about 500 mg/dose, lower than about 450 mg/dose, lower than about 400 mg/dose, lower than about 350 mg/dose, lower than about 300 mg/dose, lower than about 250 mg/dose, lower than about 200 mg/dose, lower than about 175 mg/dose, lower than about 150 mg/dose, lower than about 125 mg/dose, lower than about 100 mg/dose, lower than about 90 mg/dose, lower than about 80 mg/dose, lower than about 70 mg/dose, lower than about 60 mg/dose, lower than about 50 mg/dose, lower than about 40 mg/dose, lower than about 30 mg/dose, lower than about 20 mg/dose, or lower than about 10 mg/dose.

In some aspects, the ASO (e.g., ASO1 or nusinersen, an analog thereof, a variant thereof, a derivative thereof, or a combination thereof) can be administered at a dose lower than about 12 mg/dose/day, lower than about 11 mg/dose/day, lower than about 10 mg/dose/day, lower than about 9 mg/dose/day, lower than about 8 mg/dose/day, lower than about 7 mg/dose/day, lower than about 6 mg/dose/day, lower than about 5 mg/dose/day, lower than about 4 mg/dose/day, lower than about 3 mg/dose/day, lower than about 2 mg/dose/day, or lower than about 1 mg/dose/day; and the histone deacetylase inhibitor (e.g., valproic acid, trichostatin A, or a combination thereof) can be administered at a dose lower than about 600 mg/dose/day, lower than about 550 mg/dose/day, lower than about 500 mg/dose/day, lower than about 550 mg/dose/day, lower than about 500 mg/dose/day, lower than about 450 mg/dose/day, lower than about 400 mg/dose/day, lower than about 350 mg/dose/day, lower than about 300 mg/dose/day, lower than about 250 mg/dose/day, lower than about 200 mg/dose/day, lower than about 175 mg/dose/day, lower than about 150 mg/dose/day, lower than about 125 mg/dose/day, lower than about 100 mg/dose/day, lower than about 90 mg/dose/day, lower than about 80 mg/dose/day, lower than about 70 mg/dose/day, lower than about 60 mg/dose/day, lower than about 50 mg/dose/day, lower than about 40 mg/dose/day, lower than about 30 mg/dose/day, lower than about 20 mg/dose/day, or lower than about 10 mg/dose/day.

In some aspects, the administration of the compositions disclose herein (e.g., a first composition comprising a splicing-modulatory ASO drug such as ASO1, nusinersen, an analog thereof, a variant thereof, a derivative thereof, or any ASO targeting SMN2 mRNA disclosed in the present specification; and a second composition comprising a histone deacetylase inhibitor such as, e.g., valproic acid, trichostatin A, or a combination thereof, where the first and second composition can be administered together or separately) to a subject with SMA according to the methods disclosed here (e.g., prior, concurrent, or sequential administration of the first and second composition) can result in an increase in inclusion of exon 7 of SMN2, an increase in the expression of SMN2 protein with exon 7, a decrease in the expression of SMN2 protein without exon 7, or a combination thereof.

In some aspects, the administration of a composition disclosed herein (e.g., a first composition comprising a splicing-modulatory ASO drug such as ASO1 or nusinersen, an analog thereof, a variant thereof, a derivative thereof, or any ASO targeting SMN2 mRNA disclosed in the present specification; and a second composition comprising a histone deacetylase inhibitor such as, e.g., valproic acid, trichostatin A, or a combination thereof, where the first and second composition can be administered together or separately) to a subject with SMA according to the methods disclosed here (e.g., prior, concurrent, or sequential administration of the first and second composition) can result in an increase in inclusion of exon 7 of SMN2 of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to a subject not treated with a composition disclosed herein.

In some aspects, the administration of a composition disclosed herein (e.g., a first composition comprising a splicing-modulatory ASO drug such as ASO1 or nusinersen, an analog thereof, a variant thereof, a derivative thereof, or any ASO targeting SMN2 mRNA disclosed in the present specification; and a second composition comprising a histone deacetylase inhibitor such as, e.g., valproic acid, trichostatin A, or a combination thereof, where the first and second composition can be administered together or separately) to a subject with SMA according to the methods disclosed here (e.g., prior, concurrent, or sequential administration of the first and second composition) can result in an increase in the expression of SMN2 protein with exon 7 of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to a subject not treated with a composition disclosed herein.

In some aspects, the administration of a composition disclosed herein (e.g., a first composition comprising a splicing-modulatory ASO drug such as ASO1 or nusinersen, a an analog thereof, variant thereof, a derivative thereof, or any ASO targeting SMN2 mRNA disclosed in the present specification; and a second composition comprising a histone deacetylase inhibitor such as, e.g., valproic acid, trichostatin A, or a combination thereof, where the first and second composition can be administered together or separately) to a subject with SMA according to the methods disclosed here (e.g., prior, concurrent, or sequential administration of the first and second composition) can result in a decrease in the expression of SMN2 protein without exon 7 of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to a subject not treated with a composition disclosed herein.

In some aspects, the administration of the compositions disclose herein (e.g., a first composition comprising a splicing-modulatory ASO drug such as ASO1 or nusinersen, an analog thereof, a variant thereof, a derivative thereof, or any ASO targeting SMN2 mRNA disclosed in the present specification; and a second composition comprising a histone deacetylase inhibitor such as, e.g., valproic acid, trichostatin A, or a combination thereof, where the first and second composition can be administered together or separately) to a subject with SMA according to the methods disclosed here (e.g., prior, concurrent, or sequential administration of the first and second composition) can result in an increase in time of survival, increase in body mass, increase in muscle coordination, improvement in neuromuscular function, or any combination thereof.

In some aspects, the administration of a composition disclosed herein (e.g., a first composition comprising a splicing-modulatory ASO drug such as ASO1 or nusinersen, an analog thereof, a variant thereof, a derivative thereof, or any ASO targeting SMN2 mRNA disclosed in the present specification; and a second composition comprising a histone deacetylase inhibitor such as, e.g., valproic acid, trichostatin A, or a combination thereof, where the first and second composition can be administered together or separately) to a subject with SMA according to the methods disclosed here (e.g., prior, concurrent, or sequential administration of the first and second composition) can result in an increase in time of survival of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to a subject not treated with a composition disclosed herein.

In some aspects, the administration of a composition disclosed herein (e.g., a first composition comprising a splicing-modulatory ASO drug such as ASO1 or nusinersen, an analog thereof, a variant thereof, a derivative thereof, or any ASO targeting SMN2 mRNA disclosed in the present specification; and a second composition comprising a histone deacetylase inhibitor such as, e.g., valproic acid, trichostatin A, or a combination thereof, where the first and second composition can be administered together or separately) to a subject with SMA according to the methods disclosed here (e.g., prior, concurrent, or sequential administration of the first and second composition) can result in an increase in body mass of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to a subject not treated with a composition disclosed herein.

In some aspects, the administration of a composition disclosed herein (e.g., a first composition comprising a splicing-modulatory ASO drug such as ASO1 or nusinersen, an analog thereof, a variant thereof, a derivative thereof, or any ASO targeting SMN2 mRNA disclosed in the present specification; and a second composition comprising a histone deacetylase inhibitor such as, e.g., valproic acid, trichostatin A, or a combination thereof, where the first and second composition can be administered together or separately) to a subject with SMA according to the methods disclosed here (e.g., prior, concurrent, or sequential administration of the first and second composition) can result in an increase in muscle coordination of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to a subject not treated with a composition disclosed herein.

In some aspects, the administration of a composition disclosed herein (e.g., a first composition comprising a splicing-modulatory ASO drug such as ASO1 or nusinersen, an analog thereof, a variant thereof, a derivative thereof, or any ASO targeting SMN2 mRNA disclosed in the present specification; and a second composition comprising a histone deacetylase inhibitor such as, e.g., valproic acid, trichostatin A, or a combination thereof, where the first and second composition can be administered together or separately) to a subject with SMA according to the methods disclosed here (e.g., prior, concurrent, or sequential administration of the first and second composition) can result in an improvement in neuromuscular function of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% with respect to a subject not treated with a composition disclosed herein.

In some aspects, the compositions of the present disclosure are administered to the subject by intrathecal administration. In some aspects, the compositions of the present disclosure are administered via an injection into the spinal canal, or into the subarachnoid space so that it reaches the cerebrospinal fluid (CSF). In some aspects, the compositions of the present disclosure are administered by lumbar puncture. In some aspects, e.g., to achieve a systemic effects (e.g., to target peripherally expressed SMN2), the compositions of the present disclosure can be administered intravenously, e.g., via bolus injection or intravenous infusion.

In some aspects, a composition of the present disclosure comprises one or more components of a combination treatment disclosed herein, e.g., in some aspects a composition comprises ASO1, nusinersen, a nusinersen analog, a nusinersen variant, a nusinersen derivative, or another splicing-modulatory drug, and a second composition comprises a histone deacetylase inhibitor (e.g., valproic acid, trichostatin A, or combination thereof; e.g., a subclinical dose). In certain aspects, when the compositions of the present disclosure are in two separate compositions, such compositions can be administered concurrently and/or consecutively. In some aspects, the compositions of the present disclosure are co-administered with one or more additional therapeutic agents (e.g., a methylation inhibitor such as 5-azacytidine).

In some aspects, the composition of the present disclosure, e.g., e.g., ASO1, nusinersen, a nusinersen analog, a nusinersen variant, a nusinersen derivative, or another splicing-modulatory drug in combination with a histone deacetylase inhibitor (e.g., valproic acid, trichostatin A, or combination thereof; e.g., a subclinical dose), and the one or more additional therapeutic agents (e.g., a methylation inhibitor such as 5-azacytidine) can be administered in the same composition.

In some aspects, the composition of the present disclosure, e.g., a composition comprising ASO1 or nusinersen, a nusinersen analog, a nusinersen variant, a nusinersen derivative, or another splicing-modulatory drug in combination with a histone deacetylase inhibitor (e.g., valproic acid, trichostatin A, or combination thereof; e.g., a subclinical dose) is administered prior to the administration of the additional therapeutic agent(s) (e.g., a methylation inhibitor such as 5-azacytidine).

In some aspects, the composition of the disclosure, e.g., a composition comprising ASO1 or nusinersen, a nusinersen analog, a nusinersen, a nusinersen derivative, or another splicing-modulatory drug in combination with a histone deacetylase inhibitor (e.g., valproic acid, trichostatin A, or combination thereof; e.g., a subclinical dose) is administered after the administration of the additional therapeutic agent(s) (e.g., a methylation inhibitor such as 5-azacytidine).

In other aspects, the composition of the disclosure, e.g., a composition comprising ASO1, nusinersen, a nusinersen analog, a nusinersen variant, a nusinersen derivative, or another splicing-modulatory drug in combination with a histone deacetylase inhibitor (e.g., valproic acid, trichostatin A, or combination thereof; e.g., a subclinical dose) is administered after the administration of the additional therapeutic agent(s) (e.g., a methylation inhibitor such as 5-azacytidine).

In further aspects, the composition of the disclosure, e.g., a composition comprising ASO1, nusinersen, a nusinersen analog, a nusinersen variant, a nusinersen derivative, or another splicing-modulatory drug in combination with a histone deacetylase inhibitor (e.g., valproic acid, trichostatin A, or combination thereof; e.g., a subclinical dose) is administered concurrently with the additional therapeutic agent(s) (e.g., a methylation inhibitor such as 5-azacytidine).

In some aspects, the subject is a human.

In some aspects, the compositions and methods disclosed herein, e.g., for the treatment of SMA further comprise at least one additional active agent or supportive therapy selected from the group consisting of SMN1 gene replacement (e.g., AVXS-101 gene therapy) SMN2 alternative splicing modulation (e.g., branaplam, RG7916, RG3039, PTK-SMA1, RG7800, sodium orthovanadate, and aclarubicin), SMN2 gene activation (e.g., salbutamol, butyrates, hydroxycarbamide), benzamide M344, hydroxamic acids (e.g., CBHA, SBHA, entinostat, panobinostat, vorinostat), prolactin, natural polyphenol compounds (e.g., resveratrol and curcumin), p38 pathway activators (e.g., celecoxib), SMN stabilization (e.g., aminoglycosides and indoprofen), neuroprotection (e.g., olesoxime, thyrotropin-releasing hormone, riluzole, beta-lactam antibiotics (e.g., ceftriaxone), muscle restoration (e.g., CK-2127107), and any combination thereof.

In some aspects of the present disclosure, a human dose is calculated or estimated from data from animal experiments, such as those described herein. In some aspects, a human dose is calculated or estimated from data from monkey and/or mouse experiments, such as those described herein. In some aspects, a human dose is calculated or estimated from data from mouse experiments, such as those described herein. In some aspects, appropriate human doses can be calculated using pharmacokinetic data from mouse along with knowledge of brain weight and/or cerebrospinal fluid (CSF) turnover rates. For example, the mouse brain weight is approximately 0.4 g, which is approximately 2% of its body weight. In humans, the average brain weight is 1.5 kg, which is approximately 2.5% of body weight. In some aspects, administration into the CSF results in elimination of a portion of the compound through uptake in brain tissue and subsequent metabolism. By using the ratio of human to mouse brain weight as a scaling factor an estimate of the elimination and clearance through the brain tissue can be calculated. Additionally, the CSF turnover rate can be used to estimate elimination of compound from the CSF to blood. Mouse CSF turnover rate is approximately 10-12 times per day (0.04 mL produced at 0.325 µl/min). Human CSF turnover rate is approximately 4 times per day (100-160 mL produced at 350 - 400 µl/min). Clearance, and therefore dosing requirements, can be based on brain weight elimination scaling, and/or the CSF turnover scaling. The extrapolated human CSF clearance can be used to estimate equivalent doses in humans that approximate doses in mice. In this way, human doses can be estimated that account for differences in tissue metabolism based on brain weight and CSF turnover rates. Such methods of calculation and estimate are known to those skilled in the art.

In some aspects, an equivalent human dose can be estimated from a desired mouse dose by multiplying the mg/kg mouse dose by a factor from about 0.25 to about 1.25 depending on the determined clearance and elimination of a particular compound. Thus, for example, in some aspects, a human dose equivalent of a 0.01 mg dose for a 20 g mouse will range from about 8.75 mg to about 43.75 mg total dose for a 70 kg human. Likewise, in some aspects, a human dose equivalent of a 0.01 mg dose for a 4 g newborn mouse will range from about 1.9 mg to about 9.4 mg total dose for a 3 kg newborn human. These example doses are merely to illustrate how one of skill may determine an appropriate human dose, and are not intended to limit the present disclosure.

In some aspects, a human dose for systemic delivery (whether administered alone or in combination with CSF delivery) is calculated or estimated from data from animal experiments, such as those described herein. Typically, an appropriate human dose (in mg/kg) for systemic dose is between 0.1 and 10 times an effective dose in animals. Thus, solely for example, a subcutaneous dose of 50 µg in a 2 g newborn mouse is a dose of 25 mg/kg. The corresponding dose for a human is predicted to be between 2.5 mg/kg and 250 mg/kg. For a 3 kilogram infant, the corresponding dose is between 7.5 mg and 750 mg. For a 25 kg child, the corresponding dose is from 62.5 mg to 6250 mg.

### III. Histone Deacetylase Inhibitors

In some aspects, the compositions and methods of the present disclose comprise the administration of a histone deacetylase inhibitor or a combination thereof, e.g., at a subclinical dose. The terms “histone deacetylase inhibitor,” and “HDAC inhibitor” refer to compounds that have the ability to interact with histone deacetylase (HDAC) and inhibit its enzymatic activity. Inhibiting histone deacetylase enzyme activity means reducing or preventing the ability of histone deacetylase to remove acetyl groups from lysine residues histones that would otherwise lead to the formation of a condensed and transcriptionally silenced chromatin. In some embodiments, such a reduction in histone deacetylase activity is at least about 50%, more preferably at least about 75%, and even more preferably at least about 90%. In another preferred embodiment, the histone deacetylase activity is reduced by at least 95%, more preferably at least 99%.

Representative HDAC inhibitors include, for example, valproic acid, trichostatin A, butyric acid, MS-27-275, SAHA, apicidin, oxanflatin, FK228, and trapoxin. Based on their structure, these inhibitors are short-chain fatty acids (e.g., butyrates such as phenylbutyrate, and valproic acid), hydroxamic acids (e.g., trichostatin A, vorinostat (SAHA), belinostat (PXD101), LAQ824, and panobinostat (LBH589)), cyclic tetrapeptides (e.g., depsipeptide and trapoxin B), benzamides (e.g., MS-27-275, 49CI944, and mocetinostat (MGCD0103)), letrophilic ketones, epoxide-containing agents (e.g., trapoxins such as trapoxin A), nicotinamide, derivatives of NAD, dihydrocoumarin, naphthopyranone, and 2-hydroxynaphthaldehydes.

Most HDAC inhibitors reversibly inhibit HDACs, with the exception of trapoxins, which have an epoxy group capable of irreversibly alkylating HDACs. Reversible inhibitors generally have a long aliphatic tail containing a nucleophilic terminus such as —SH or —OH that interacts with an active zinc center located at the bottom of the HDAC binding pocket. Other HDAC inhibitors are emerging. Most of the new agents are derivatives of hydroxamic acids, including amide analogs of trichostatin A (TSA) and thio/phosphorus based SAHA. Replacing the amide bond in the MS-27-275 structure with a sulfonamide has led to the discovery of a new class of potent HDAC inhibitors. HDAC inhibitors that have entered clinical trials include the hydroxamic acid derivative LAQ824, the butyric acid derivative Titan, valproic acid, MS-27-275, SAHA, and the depsipeptide FK228.

In some aspects of the present disclosure, the HDAC inhibitor is valproic acid, a salt thereof, or a combination thereof. In some aspects of the present disclosure, the HDAC inhibitor is valproic acid, a salt thereof, or a combination thereof at a subclinical dose. In some aspects of the present disclosure, the HDAC inhibitor is trichostatin A. In some aspects of the present disclosure, the HDAC inhibitor is trichostatin A at a subclinical dose. In some aspects, the HDAC inhibitor comprises valproic acid and trichostatin A. In some aspects, the HDAC inhibitor comprises valproic acid and trichostatin A at a subclinical dose.

### IV. Antisense Oligonucleotides (ASOs)

In some aspects, the present disclosure employs splicing-modulatory antisense oligonucleotides (ASOs) for use in modulating splicing of SMN2 mRNA in a subject. SMN2 contains a translationally silent mutation (C→T) at position +6 of exon 7, which results in inefficient inclusion of exon 7 in SMN2 transcripts. Therefore, the predominant form of SMN2, one which lacks exon 7, is unstable and inactive. Therapeutic compounds capable of modulating SMN2 splicing such that the percentage of SMN2 transcripts containing exon 7 is increased are therefore useful for the treatment of SMA.

In some aspects, the ASOs disclosed herein, e.g., ASO1, nusinersen, analogs thereof, variants thereof, or derivatives thereof, can be administered at doses below the recommended dosage. Thus, in some aspects, ASOs disclosed herein can be administered at doses that would be considered sub-optimal by a person skilled in the art.

In certain instances, the present disclosure provides antisense compounds complementary to a pre-mRNA encoding SMN2. In certain such instances, the antisense compound alters splicing of SMN2. Certain sequences and regions useful for altering splicing of SMN2 can be found, e.g., in WO 2007/002390. Other ASOs that can be used as an ASO component of a compositions disclosed herein, or in a methods disclosed herein included, for example, those disclosed in U.S. Pat. Nos. 9717750B2, 10436802B2, or 9926559B2, U.S. Publications No. 2017/0044538A1, or PCT Publ. No. WO2019075357A1, all of which are herein incorporated by reference in their entireties.

In certain aspects, the ASO used in the compositions and methods of the present disclosure has a nucleobase sequence complementary to intron 7 of SMN2. Certain such nucleobase sequences are exemplified in the following non-limiting list: ATTCACTTTCATAATGCTGG (ASO1) (SEQ ID NO: 1), TGCTGGCAGACTTAC (SEQ ID NO:58), CATAATGCTGGCAGA (SEQ ID NO:59), TCATAATGCTGGCAG (SEQ ID NO:60), TTCATAATGCTGGCA (SEQ ID NO:61), TTTCATAATGCTGGC (SEQ ID NO:62), TCACTTTCATAATGCTGG (nusinersen) (SEQ ID NO:63), AGTAAGATTCACTTT (SEQ ID NO:64), CTTTCATAATGCTGG (SEQ ID NO:65), TCATAATGCTGG (SEQ ID NO:66), ACTTTCATAATGCTG (SEQ ID NO:67), TTCATAATGCTG (SEQ ID NO:68), CACTTTCATAATGCT (SEQ ID NO:69), TTTCATAATGCT (SEQ ID NO:70), TCACTTTCATAATGC (SEQ ID NO:71), CTTTCATAATGC (SEQ ID NO:72), TTCACTTTCATAATG (SEQ ID NO:73), ACTTTCATAATG (SEQ ID NO:74), ATTCACTTTCATAAT (SEQ ID NO:75), CACTTTCATAAT (SEQ ID NO:76), GATTCACTTTCATAA (SEQ ID NO:77), TCACTTTCATAA (SEQ ID NO:78), TTCACTTTCATA (SEQ ID NO:79), and ATTCACTTTCAT (SEQ ID NO:80).

In some aspects, an ASO used in the compositions and methods of the present disclosure, comprises at least 5, 6, 7, 8, 9, 10, 11, 12, 13, 14, 15, 16, 17, 18, 19, or 20 nucleobases from a sequence selected from the group consisting of SEQ ID NOS: 1, and 58-80. In some aspects, an ASO used in the compositions and methods of the present disclosure, comprises about 5, 6, 7, 8, 9, 10, 11, 12, 13, 14, 15, 16, 17, 18, 19, or 20 nucleobases from a sequence selected from the group consisting of SEQ ID NOS: 1, and 58-80. In some aspects, an ASO used in the compositions and methods of the present disclosure, comprises consist or consists essentially of 5, 6, 7, 8, 9, 10, 11, 12, 13, 14, 15, 16, 17, 18, 19, or 20 nucleobases from a sequence selected from the group consisting of SEQ ID NOS: 1, and 58-80.

In some aspects, the ASO used in the compositions and methods of the present disclosure, has at least about 80% sequence identity to a sequence selected from the group consisting of SEQ ID NOS: 1, and 58-80, such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity.

In some aspects, the ASO used in the compositions and methods of the present disclosure is selected from, or comprises, one of the sequences selected from the group consisting of SEQ ID NOS: 1, and 58-80 or a region of at least 10 contiguous nucleotides thereof, wherein the ASO (or contiguous nucleotide portion thereof) can optionally comprise one, two, three, or four mismatches when compared to the corresponding sequence in the SMN2 mRNA.

In some aspects, the ASO used in the compositions and methods of the present disclosure comprises, consists, or consists essentially of the sequence as set forth in SEQ ID NO: 1, or an analog, variant, or derivative thereof. In aspects, the ASO used in the compositions and methods of the present disclosure comprises an ASO of SEQ ID NO:1, or an analog, variant, or derivative thereof, and a second ASO combined.

In some aspects, the ASO used in the compositions and methods of the present disclosure comprises, consists, or consists essentially of the sequence as set forth in SEQ ID NO: 58, or an analog, variant, or derivative thereof. In aspects, the ASO used in the compositions and methods of the present disclosure comprises, consists, or consists essentially of the sequence as set forth in SEQ ID NO: 59, or an analog, variant, or derivative thereof. In aspects, the ASO used in the compositions and methods of the present disclosure comprises, consists, or consists essentially of the sequence as set forth in SEQ ID NO: 60, or an analog, variant, or derivative thereof. In aspects, the ASO used in the compositions and methods of the present disclosure comprises, consists, or consists essentially of the sequence as set forth in SEQ ID NO: 61, or an analog, variant, or derivative thereof. In aspects, the ASO used in the compositions and methods of the present disclosure comprises, consists, or consists essentially of the sequence as set forth in SEQ ID NO: 62, or an analog, variant, or derivative thereof. In aspects, the ASO used in the compositions and methods of the present disclosure comprises, consists, or consists essentially of the sequence as set forth in SEQ ID NO: 63, or an analog, variant, or derivative thereof. In aspects, the ASO used in the compositions and methods of the present disclosure comprises, consists, or consists essentially of the sequence as set forth in SEQ ID NO: 64, or an analog, variant, or derivative thereof. In aspects, the ASO used in the compositions and methods of the present disclosure comprises, consists, or consists essentially of the sequence as set forth in SEQ ID NO: 65, or an analog, variant, or derivative thereof. In aspects, the ASO used in the compositions and methods of the present disclosure comprises, consists, or consists essentially of the sequence as set forth in SEQ ID NO: 66, or an analog, variant, or derivative thereof. In aspects, the ASO used in the compositions and methods of the present disclosure comprises, consists, or consists essentially of the sequence as set forth in SEQ ID NO: 67, or an analog, variant, or derivative thereof. In aspects, the ASO used in the compositions and methods of the present disclosure comprises, consists, or consists essentially of the sequence as set forth in SEQ ID NO: 68, or an analog, variant, or derivative thereof. In aspects, the ASO used in the compositions and methods of the present disclosure comprises, consists, or consists essentially of the sequence as set forth in SEQ ID NO: 69, or an analog, variant, or derivative thereof. In aspects, the ASO used in the compositions and methods of the present disclosure comprises, consists, or consists essentially of the sequence as set forth in SEQ ID NO: 70, or an analog, variant, or derivative thereof. In aspects, the ASO used in the compositions and methods of the present disclosure comprises, consists, or consists essentially of the sequence as set forth in SEQ ID NO: 71, or an analog, variant, or derivative thereof. In aspects, the ASO used in the compositions and methods of the present disclosure comprises, consists, or consists essentially of the sequence as set forth in SEQ ID NO: 72, or an analog, variant, or derivative thereof. In aspects, the ASO used in the compositions and methods of the present disclosure comprises, consists, or consists essentially of the sequence as set forth in SEQ ID NO: 73, or an analog, variant, or derivative thereof. In aspects, the ASO used in the compositions and methods of the present disclosure comprises, consists, or consists essentially of the sequence as set forth in SEQ ID NO: 74, or an analog, variant, or derivative thereof. In aspects, the ASO used in the compositions and methods of the present disclosure comprises, consists, or consists essentially of the sequence as set forth in SEQ ID NO: 75, or an analog, variant, or derivative thereof. In aspects, the ASO used in the compositions and methods of the present disclosure comprises, consists, or consists essentially of the sequence as set forth in SEQ ID NO: 76, or an analog, variant, or derivative thereof. In aspects, the ASO used in the compositions and methods of the present disclosure comprises, consists, or consists essentially of the sequence as set forth in SEQ ID NO: 77, or an analog, variant, or derivative thereof. In aspects, the ASO used in the compositions and methods of the present disclosure comprises, consists, or consists essentially of the sequence as set forth in SEQ ID NO: 78, or an analog, variant, or derivative thereof. In aspects, the ASO used in the compositions and methods of the present disclosure comprises, consists, or consists essentially of the sequence as set forth in SEQ ID NO: 79, or an analog, variant, or derivative thereof. In aspects, the ASO used in the compositions and methods of the present disclosure comprises, consists, or consists essentially of the sequence as set forth in SEQ ID NO: 80, or an analog, variant, or derivative thereof.

In some aspects, the ASO consists of the sequence as set forth in SEQ ID NO: 1 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 58 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 59 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 60 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 61 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 62 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 63 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 64 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 65 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 66 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 67 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 68 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 69 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 70 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 71 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 72 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 73 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 74 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 75 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 76 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 77 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 78 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 79 plus 1, 2, 3, 4, or 5 additional 5′ nucleotides complementary to the corresponding sequence in the SMN2 mRNA (see SEQ ID NO:57). In some aspects, the ASO consists of the sequence as set forth in SEQ ID NO: 80 plus 1, 2, 3, 4, or 5 additional 5′ nucleotides complementary to the corresponding sequence in the SMN2 mRNA (see SEQ ID NO:57).

In some aspects, the ASO consists of the sequence as set forth in SEQ ID NO: 1 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 58 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 59 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 60 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 61 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 62 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 63 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 64 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 65 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 66 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 67 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 68 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 69 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 70 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 71 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 72 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 73 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 74 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 75 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 76 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 77 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 78 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 79 plus 1, 2, 3, 4, or 5 additional 3′ nucleotides complementary to the corresponding sequence in the SMN2 mRNA (see SEQ ID NO:57). In some aspects, the ASO consists of the sequence as set forth in SEQ ID NO: 80 plus 1, 2, 3, 4, or 5 additional 3′ nucleotides complementary to the corresponding sequence in the SMN2 mRNA (see SEQ ID NO:57).

In some aspects, the ASO consists of the sequence as set forth in SEQ ID NO: 1 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 58 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 59 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 60 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 61 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 62 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 63 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 64 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 65 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 66 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 67 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 68 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 69 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 70 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 71 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 72 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 73 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 74 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 75 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 76 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 77 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 78 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 79 plus 1, 2, 3, 4, or 5 additional 5′ and/or 3′ nucleotides complementary to the corresponding sequence in the SMN2 mRNA (see SEQ ID NO:57). In some aspects, the ASO consists of the sequence as set forth in SEQ ID NO: 80 plus 1, 2, 3, 4, or 5 additional 5′ and/or 3′ nucleotides complementary to the corresponding sequence in the SMN2 mRNA (see SEQ ID NO:57).

In some aspects, the ASO of the disclosure binds to the target nucleic acid sequence (e.g., a SMN2 mRNA transcript) and is capable of increasing the level of inclusion of an exon, e.g., exon 7 of SMN2. In some aspects, the inclusion of the exon e.g., exon 7 of SMN2, provides an mRNA that encodes a protein that is less associated or not associated with a condition, disease, or disorder, (e.g., SMA or ALS) compared to a protein encoded by a corresponding mRNA which does not include the exon but otherwise has the same exons. In some aspects, the exon included is exon 7 of SMN2.

In some aspects, administration of an ASO of the disclosure increases the inclusion of exon 7 of SMN2 by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% compared to the exon 7 inclusion level in the absence of treatment with the ASO, or a composition comprising the ASO (e.g., a composition of the present disclosure comprising an ASO disclosed herein and a histone deacetylase inhibitor, e.g., valproic acid, e.g., at a subclinical dose).

In some aspects, administration of an ASO of the disclosure increases the expression level of an SMN2 protein comprising the amino acid sequence encoded by exon 7 of SMN2 by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% compared to the expression level of the SMN2 protein comprising the amino acid sequence encoded by exon 7 of SMN2 exon 7 in the absence of treatment with the ASO or a composition comprising the ASO (e.g., a composition of the present disclosure comprising an ASO disclosed herein and a histone deacetylase inhibitor, e.g., valproic acid, e.g., at a subclinical dose).

In some aspects, administration of an ASO of the disclosure increases the expression level of an SMN2 protein comprising the amino acid sequence encoded by exon 7 of SMN2 by at least about 1-fold, at least about 2-fold, at least about 3-fold, at least about 4-fold, at least about 5-fold, at least about 6-fold, at least about 7-fold, at least about 8-fold, at least about 9-fold, or at least about 1-fold compared to the expression level of the SMN2 protein comprising the amino acid sequence encoded by exon 7 of SMN2 exon 7 in the absence of treatment with the ASO, or a composition comprising the ASO (e.g., a composition of the present disclosure comprising an ASO disclosed herein and a histone deacetylase inhibitor, e.g., valproic acid, e.g., at a subclinical dose).

In some aspects, the ASO can tolerate 1, 2, 3, or 4 (or more) mismatches, when hybridizing to the target sequence and still sufficiently bind to the target to show the desired effect, i.e., inclusion of exon 7 of SMN2, and therefore an increase in the expression levels of SMN2 protein comprising the amino acid sequence encoded by exon 7. Mismatches can be compensated, for example, by increased length of the ASO nucleotide sequence and/or an increased number of nucleotide analogs, which are disclosed elsewhere herein.

In some aspects, the ASO of the disclosure comprises no more than three mismatches when hybridizing to the target sequence. In other aspects, the contiguous nucleotide sequence comprises no more than two mismatches when hybridizing to the target sequence. In other aspects, the contiguous nucleotide sequence comprises no more than one mismatch when hybridizing to the target sequence.

In some aspects, the ASO of the disclosure comprises a moiety capable of binding to ASGPR (Asialoglycoprotein Receptor). In some aspects, the present disclosure provides an ASO conjugated to a moiety capable of binding to an ASGP receptor in the brain. In some aspects, the moiety capable of binding to an ASGPR is a ASGPR ligand. In some aspects, the moiety capable of binding to an ASGPR is lactose, galactose, N-acetylgalactosamine (GalNAc), galactosamine, N-formylgalactosamine, N- propionylgalactosamine, N-n-butanoyl-galactosamine, or N-iso-butanoyl-galactosamine. In some aspects, the moiety capable of binding to a ASGP receptor is carbohydrate. In some aspects, the moiety capable of binding to a ASGP receptor is GalNAc or a derivative thereof. In some aspects, the GalNAc moiety comprises, e.g., GalNAc or a variant or derivative thereof, as described, e.g., in any of: Migawa et al.2016 Bioorg. Med. Chem. Lett.26: 2914-7; Ostergaard et al.2015 Bioconjug. Chem.26: 1451-1455; Prakash et al.2014 Nucl. Acids Res. 42: 8796-8807; Prakash et al.2016 J. Med. Chem.59: 2718-33; Shemesh et al.2016 Mol. Ther. Nucl. Acids 5: e319; St-Pierre et al.2016 Bioorg. Med. Chem.24: 2397-409; and/or Yu et al. 2016 Mol. Ther. Nucl. Acids 5: e317, all of which are herein incorporated by reference in their entireties.

### IV.A. ASO Length

The ASOs of the present disclosure (e.g., ASO1, nusinersen, and analog thereof, a variant thereof, a derivative thereof, or a combination thereof) can comprise a contiguous nucleotide sequence of a total of 5, 6, 7, 8, 9, 10, 11, 12, 13, 14, 15, 16, 17, 18, 19, 20, 21, 22, 23, 24, 25, 26, 27, 28, 29, or 30 contiguous nucleotides in length. It should be understood that when a range is given for an ASO, or contiguous nucleotide sequence length, the range includes the lower and upper lengths provided in the range, for example from (or between) 10-30, includes both 10 and 30.

In some aspects, the ASO of the present disclosure (e.g., ASO1, nusinersen, and analog thereof, a variant thereof, a derivative thereof, or a combination thereof) comprises a contiguous nucleotide sequence with a total of about 14 to about 20, i.e., about 14, about 15, about 16, about 17, about 18, about 19, or about 20 contiguous nucleotides in length. In some aspects, the ASO of the present disclosure (e.g., ASO1, nusinersen, and analog thereof, a variant thereof, a derivative thereof, or a combination thereof) comprises a contiguous nucleotide sequence of a total of about 20 contiguous nucleotides in length. In some aspects, the ASO of the present disclosure is 14 nucleotides in length. In some aspects, the ASO of the present disclosure is 15 nucleotides in length. In some aspects, the ASO of the present disclosure is 16 nucleotides in length. In some aspects, the ASO of the present disclosure is 17 nucleotides in length. In some aspects, the ASO of the present disclosure is 18 nucleotides in length. In some aspects, the ASO of the present disclosure is 19 nucleotides in length. In some aspects, the ASOs of the present disclosure is 20 nucleotides in length.

In some aspects, the ASO of the present disclosure (e.g., ASO1, nusinersen, and analog thereof, a variant thereof, a derivative thereof, or a combination thereof) comprises or consists of X to Y linked nucleosides, where X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, in some aspects, the disclosure provides ASOs comprising or consisting of: 8 to 9, 8 to 10, 8 to 11, 8 to 12, 8 to 13, 8 to 14, 8 to 15, 8 to 16, 8 to 17, 8 to 18, 8 to 19, 8 to 20, 8 to 21, 8 to 22, 8 to 23, 8 to 24, 8 to 25, 8 to 26, 8 to 27, 8 to 28, 8 to 29, 8 to 30, 9 to 10, 9 to 11, 9 to 12, 9 to 13, 9 to 14, 9 to 15, 9 to 16, 9 to 17, 9 to 18, 9 to 19, 9 to 20, 9 to 21, 9 to 22, 9 to 23, 9 to 24, 9 to 25, 9 to 26, 9 to 27, 9 to 28, 9 to 29, 9 to 30, 10 to 11, 10 to 12, 10 to 13, 10 to 14, 10 to 15, 10 to 16, 10 to 17, 10 to 18, 10 to 19, 10 to 20, 10 to 21, 10 to 22, 10 to 23, 10 to 24, 10 to 25, 10 to 26, 10 to 27, 10 to 28, 10 to 29, 10 to 30, 11 to 12, 11 to 13, 11 to 14, 11 to 15, 11 to 16, 11 to 17, 11 to 18, 11 to 19, 11 to 20, 11 to 21, 11 to 22, 11 to 23, 11 to 24, 11 to 25, 11 to 26, 11 to 27, 11 to 28, 11 to 29, 11 to 30, 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### IV.B. Nucleosides and Nucleoside Analogs

In one aspect, the ASOs of the present disclosure (e.g., ASO1, nusinersen, and analog thereof, a variant thereof, a derivative thereof, or a combination thereof) comprise one or more non-naturally occurring nucleoside analogs. “Nucleoside analogs” as used herein are variants of natural nucleosides, such as DNA or RNA nucleosides, by virtue of modifications in the sugar and/or base moieties. In principle, analogs could be merely “silent” or “equivalent” to the natural nucleosides in the context of the oligonucleotide, i.e. have no functional effect on the way the oligonucleotide works to inhibit target gene expression. Such “equivalent” analogs can nevertheless be useful if, for example, they are easier or cheaper to manufacture, or are more stable to storage or manufacturing conditions, or represent a tag or label. In some aspects, however, the analogs will have a functional effect on the way in which the ASO works to inhibit expression; for example by producing increased binding affinity to the target and/or increased resistance to intracellular nucleases and/or increased ease of transport into the cell. Specific examples of nucleoside analogs are described by e.g. Freier & Altmann; Nucl. Acid Res., 1997, 25, 4429-4443 and Uhlmann; Curr. Opinion in Drug Development, 2000, 3(2), 293-213, and in Scheme 1. The ASOs of the present disclosure can contain more than one, more than two, more than three, more than four, more than five, more than six, more than seven, more than eight, more than nine, more than 10, more than 11, more than 12, more than 13, more than 14, more than 15, more than 16, more than 18, more than 19, or more than 20 nucleoside analogs. In some aspects, the nucleoside analogs in the ASOs are the same. In other aspects, the nucleoside analogs in the ASOs are different. The nucleotide analogs in the ASOs can be any one of or combination of the following nucleoside analogs.

In some aspects, the nucleoside analog comprises a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; bicyclic nucleoside analog; or any combination thereof. In some aspects, the nucleoside analog comprises a sugar-modified nucleoside. In some aspects, the nucleoside analog comprises a nucleoside comprising a bicyclic sugar. In some aspects, the nucleoside analog comprises an LNA.

In some aspects, the nucleoside analog is selected from the group consisting of constrained ethyl nucleoside (cEt), 2′,4′-constrained 2′-O-methoxyethyl (cMOE), α-L-LNA, β-D-LNA, 2′-O,4′-C-ethylene-bridged nucleic acids (ENA), amino-LNA, oxy-LNA, thio-LNA, and any combination thereof. In some aspects, the ASO comprises one or more 5′-methyl-cytosine nucleobases.

### IV.B.1. Nucleobase

The term nucleobase includes the purine (e.g., adenine and guanine) and pyrimidine (e.g., uracil, thymine and cytosine) moiety present in nucleosides and nucleotides that form hydrogen bonds in nucleic acid hybridization. In the context of the present disclosure, the term nucleobase also encompasses modified nucleobases that can differ from naturally occurring nucleobases, but are functional during nucleic acid hybridization. In some aspects, the nucleobase moiety is modified by modifying or replacing the nucleobase. In this context, “nucleobase” refers to both naturally occurring nucleobases such as adenine, guanine, cytosine, thymidine, uracil, xanthine and hypoxanthine, as well as non-naturally occurring variants. Such variants are for example described in Hirao et al., (2012) Accounts of Chemical Research vol 45 page 2055 and Bergstrom (2009) Current Protocols in Nucleic Acid Chemistry Suppl. 37 1.4.1.

In a some aspects, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl-cytosine, 5-thiozolo-cytosine, 5-propynyl-cytosine, 5-propynyl-uracil, 5-bromouracil, 5-thiazolo-uracil, 2-thio-uracil, 2′thio-thymine, inosine, diaminopurine, 6-aminopurine, 2-aminopurine, 2,6-diaminopurine, and 2-chloro-6-aminopurine.

The nucleobase moieties can be indicated by the letter code for each corresponding nucleobase, e.g., A, T, G, C, or U, wherein each letter can optionally include modified nucleobases of equivalent function. For example, in the exemplified oligonucleotides, the nucleobase moieties are selected from A, T, G, C, and 5-methyl-cytosine. Optionally, for LNA gapmers, 5-methyl-cytosine LNA nucleosides can be used.

### IV.B.2. Sugar Modification

The ASO of the disclosure can comprise one or more nucleosides that have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2′ and C4′ carbons on the ribose ring (LNA), or an unlinked ribose ring which typically lacks a bond between the C2′ and C3′ carbons (e.g., UNA). Other sugar-modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in RNA nucleosides. Substituents can, for example be introduced at the 2′, 3′, 4′, or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

### IV.B.2.a 2′ Modified Nucleosides

A 2′ sugar modified nucleoside is a nucleoside which has a substituent other than H or -OH at the 2′ position (2′ substituted nucleoside) or comprises a 2′ linked biradical, and includes 2′ substituted nucleosides and LNA (2′ - 4′ biradical bridged) nucleosides. For example, the 2′ modified sugar can provide enhanced binding affinity (e.g., affinity enhancing 2′ sugar modified nucleoside) and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acids (ANA), and 2′-Fluoro-ANA nucleoside. For further examples, please see, e.g., Freier & Altmann; Nucl. Acid Res., 1997, 25, 4429-4443; Uhlmann, Curr. Opinion in Drug Development, 2000, 3(2), 293-213; and Deleavey and Damha, Chemistry and Biology 2012, 19, 937. Below are illustrations of some 2′ substituted modified nucleosides.

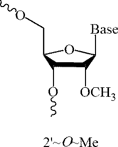

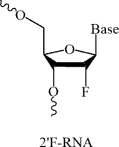

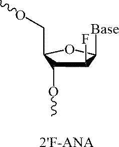

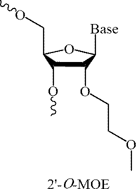

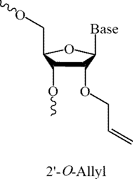

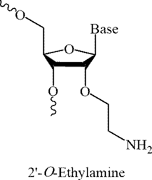

### IV.B.2.b Locked Nucleic Acid Nucleosides (LNA)

LNA nucleosides are modified nucleosides which comprise a linker group (referred to as a biradical or a bridge) between C2′ and C4′ of the ribose sugar ring of a nucleoside (i.e., 2′-4′ bridge), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med.Chem. Lett. 12, 73-76, Seth et al., J. Org. Chem. 2010, Vol 75(5) pp. 1569-81, and Mitsuoka et al., Nucleic Acids Research 2009, 37(4), 1225-1238.

In some aspects, the modified nucleoside or the LNA nucleosides of the ASO of the disclosure has a general structure of the formula I or II:

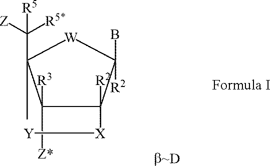

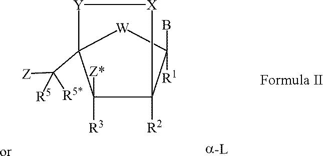

wherein
W is selected from —O—, —S—, —N(R^{a})—, —C(R^{a}R^{b})—, in particular —O—;
B is a nucleobase or a modified nucleobase moiety;
Z is an internucleoside linkage to an adjacent nucleoside or a 5′-terminal group;
Z* is an internucleoside linkage to an adjacent nucleoside or a 3′-terminal group;
R¹, R², R³, R⁵ and R^{5*} are independently selected from hydrogen, halogen, alkyl, alkenyl, alkynyl, hydroxy, alkoxy, alkoxyalkyl, alkenyloxy, carboxyl, alkoxycarbonyl, alkylcarbonyl, formyl, azide, heterocycle and aryl; and
X, Y, R^{a} and R^{b} are as defined herein.

In some aspects, —X—Y—, R^{a} is hydrogen or alkyl, in particular hydrogen or methyl. In some aspects of —X—Y—, R^{b} is hydrogen or alkyl, in particular hydrogen or methyl. In other aspects of —X—Y—, one or both of R^{a} and R^{b} are hydrogen. In further aspects of —X—Y—, only one of R^{a} and R^{b} is hydrogen. In some aspects of —X—Y—, one of R^{a} and R^{b} is methyl and the other one is hydrogen. In certain aspects of —X—Y—, R^{a} and R^{b} are both methyl at the same time.

In some aspects, —X—, R^{a} is hydrogen or alkyl, in particular hydrogen or methyl. In some aspects of —X—, R^{b} is hydrogen or alkyl, in particular hydrogen or methyl. In other aspects of —X—, one or both of R^{a} and R^{b} are hydrogen. In certain aspects of —X—, only one of R^{a} and R^{b} is hydrogen. In certain aspects of —X—, one of R^{a} and R^{b} is methyl and the other one is hydrogen. In other aspects of —X—, R^{a} and R^{b} are both methyl at the same time.

In some aspects, —Y—, R^{a} is hydrogen or alkyl, in particular hydrogen or methyl. In certain aspects of —Y—, R^{b} is hydrogen or alkyl, in particular hydrogen or methyl. In other aspects of —Y—, one or both of R^{a} and R^{b} are hydrogen. In some aspects of —Y—, only one of R^{a} and R^{b} is hydrogen. In other aspects of —Y—, one of R^{a} and R^{b} is methyl and the other one is hydrogen. In some aspects of —Y—, R^{a} and R^{b} are both methyl at the same time.

In some aspects, R¹, R², R³, R⁵ and R^{5*} are independently selected from hydrogen and alkyl, in particular hydrogen and methyl.

In some aspects, R¹, R², R³, R⁵ and R^{5*} are all hydrogen at the same time.

In some aspects, R¹, R², R³, are all hydrogen at the same time, one of R⁵ and R^{5*} is hydrogen and the other one is as defined above, in particular alkyl, more particularly methyl.

In some aspects, R¹, R², R³, are all hydrogen at the same time, one of R⁵ and R^{5*} is hydrogen and the other one is azide..

In some aspects, —X—Y— is —O—CH₂—, W is oxygen and R¹, R², R³, R⁵ and R^{5*} are all hydrogen at the same time. Such LNA nucleosides are disclosed in WO 99/014226, WO 00/66604, WO 98/039352 and WO 2004/046160, which are all hereby incorporated by reference, and include what are commonly known in the art as beta-D-oxy LNA and alpha-L-oxy LNA nucleosides.

In some aspects, —X—Y— is —S—CH₂—, W is oxygen and R¹, R², R³, R⁵ and R^{5*} are all hydrogen at the same time. Such thio LNA nucleosides are disclosed in PCT Publications WO 99/014226 and WO 2004/046160 which are hereby incorporated by reference.

In some aspects, —X—Y— is —NH—CH₂—, W is oxygen and R¹, R², R³, R⁵ and R^{5*} are all hydrogen at the same time. Such amino LNA nucleosides are disclosed in WO 99/014226 and WO 2004/046160, which are hereby incorporated by reference.

In some aspects, —X—Y— is —O—CH₂CH₂— or —OCH₂CH₂CH₂—, W is oxygen, and R¹, R², R³, R⁵ and R^{5*} are all hydrogen at the same time. Such LNA nucleosides are disclosed in WO 00/047599 and Morita et al., Bioorganic & Med.Chem. Lett. 12, 73-76, which are hereby incorporated by reference, and include what are commonly known in the art as 2′-O-4′C-ethylene bridged nucleic acids (ENA).

In some aspects, —X—Y— is —O—CH₂—, W is oxygen, R¹, R², R³ are all hydrogen at the same time, one of R⁵ and R⁵* is hydrogen and the other one is not hydrogen, such as alkyl, for example methyl. Such 5′ substituted LNA nucleosides are disclosed in WO 2007/134181, which is hereby incorporated by reference.

In some aspects, —X—Y— is —O—CR^{a}R^{b}—, wherein one or both of R^{a} and R^{b} are not hydrogen, in particular alkyl such as methyl, W is oxygen, R¹, R², R³ are all hydrogen at the same time, one of R³ and R^{5*} is hydrogen and the other one is not hydrogen, in particular alkyl, for example methyl. Such bis modified LNA nucleosides are disclosed in WO 2010/077578, which is hereby incorporated by reference.

In some aspects, —X—Y— is —O—CH(CH₂—O—CH₃)— (“2′ O-methoxyethyl bicyclic nucleic acid”, Seth et al., J. Org. Chem. 2010, Vol 75(5) pp. 1569-81).

In some aspects, —X—Y— is —O—CHR^{a}—, W is oxygen and R¹, R², R³, R⁵ and R^{5*} are all hydrogen at the same time. Such 6′-substituted LNA nucleosides are disclosed in WO 2010/036698 and WO 2007/090071, which are both hereby incorporated by reference. In such 6′-substituted LNA nucleosides, Rª is in particular C1-C6 alkyl, such as methyl.

In some aspects, —X—Y— is —O—CH(CH₂—O—CH₃)—, W is oxygen and R¹, R², R³, R⁵ and R^{5*} are all hydrogen at the same time. Such LNA nucleosides are also known in the art as cyclic MOEs (cMOE) and are disclosed in PCT Publication WO 2007/090071.

In some aspects, —X—Y— is —O—CH(CH₃)—.

In some aspects, —X—Y— is —O—CH₂—O—CH₂— (Seth et al., J. Org. Chem 2010 op. cit.)

In some aspects, —X—Y— is —O—CH(CH₃)—, W is oxygen and R¹, R², R³, R⁵ and R^{5*} are all hydrogen at the same time. Such 6′-methyl LNA nucleosides are also known in the art as cET nucleosides, and can be either (S)-cET or (R)-cET diastereoisomers, as disclosed in PCT Publications WO 2007/090071 (beta-D) and WO 2010/036698 (alpha-L) which are both hereby incorporated by reference.

In some aspects, —X—Y— is —O—CR^{a}R^{b}—, wherein neither R^{a} nor R^{b} is hydrogen, W is oxygen, and R¹, R², R³, R⁵ and R⁵* are all hydrogen at the same time. In certain aspects, R^{a} and R^{b} are both alkyl at the same time, in particular both methyl at the same time. Such 6′-disubstituted LNA nucleosides are disclosed in PCT Publication WO 2009/006478, which is hereby incorporated by reference.

In some aspects, —X—Y— is —S—CHR^{a}—, W is oxygen, and R¹, R², R³, R⁵ and R⁵* are all hydrogen at the same time. Such 6′-substituted thio LNA nucleosides are disclosed in WO 2011/156202, which is hereby incorporated by reference. In certain aspects of such 6′-substituted thio LNA, R^{a} is alkyl, in particular methyl.

In some aspects, —X—Y— is —C(═CH₂)C(R^{a}R^{b})—, such as, W is oxygen, and R¹, R², R³, R⁵ and R^{5*} are all hydrogen at the same time. Such vinyl carbo LNA nucleosides are disclosed in PCT Publications WO 2008/154401 and WO 2009/067647, which are both hereby incorporated by reference.

In some aspects, —X—Y— is —N(OR^{a})—CH₂—, W is oxygen and R¹, R², R³, R⁵ and R^{5*} are all hydrogen at the same time. In some aspects, R^{a} is alkyl such as methyl. Such LNA nucleosides are also known as N substituted LNAs and are disclosed in PCT Publication WO 2008/150729, which is hereby incorporated by reference.

In some aspects, —X—Y— is —O—NCH₃— (Seth et al., J. Org. Chem 2010 op. cit.).

In some aspects, —X—Y— is ON(R^{a})— —N(R^{a})—O—,—NR^{a}—CR^{a}R^{b}—CR^{a}R^{b}—, or —NR^{a}—CR^{a}R^{b}—, W is oxygen, and R¹, R², R³, R⁵ and R^{5*} are all hydrogen at the same time. In certain aspects, R^{a} is alkyl, such as methyl. (Seth et al., J. Org. Chem 2010 op. cit.).

In some aspects, R⁵ and R⁵* are both hydrogen at the same time. In other aspects, one of R⁵ and R^{5*} is hydrogen and the other one is alkyl, such as methyl. In such aspects, R¹, R² and R³ can be in particular hydrogen and —X—Y— can be in particular —O—CH₂— or —O—CHC(R^{a})₃—, such as —O—CH(CH₃)—.

In some aspects, —X—Y— is —CR^{a}R^{b}—O—CR^{a}R^{b}—, such as —CH₂—O—CH₂—, W is oxygen and R¹, R², R³, R⁵ and R^{5*} are all hydrogen at the same time. In such aspects, R^{a} can be in particular alkyl such as methyl. Such LNA nucleosides are also known as conformationally restricted nucleotides (CRNs) and are disclosed in PCT Publication WO 2013/036868, which is hereby incorporated by reference.

In some aspects, —X—Y— is —O—CR^{a}R^{b}—O—CR^{a}R^{b}—, such as —O—CH₂—O—CH₂—, W is oxygen and R¹, R², R³, R⁵ and R^{5*} are all hydrogen at the same time. In certain aspects, R^{a} can be in particular alkyl such as methyl. Such LNA nucleosides are also known as COC nucleotides and are disclosed in Mitsuoka et al., Nucleic Acids Research 2009, 37(4), 1225-1238, which is hereby incorporated by reference.

It will be recognized than, unless specified, the LNA nucleosides can be in the beta-D or alpha-L stereoisoform.

Certain examples of LNA nucleosides are presented in Scheme 1.

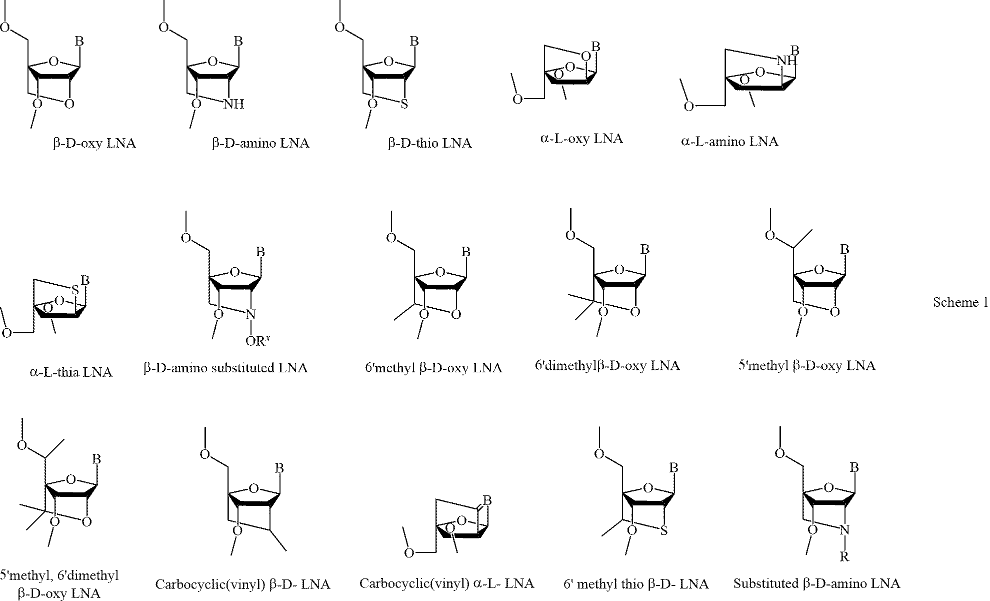

As illustrated elsewhere, in some aspects of the disclosure the LNA nucleosides in the oligonucleotides are beta-D-oxy-LNA nucleosides.

### IV.C. Nuclease Mediated Degradation

Nuclease mediated degradation refers to an oligonucleotide, e.g., an ASO of the present disclosure (e.g., ASO1, nusinersen, and analog thereof, a variant thereof, a derivative thereof, or a combination thereof), capable of mediating degradation of a complementary nucleotide sequence when forming a duplex with such a sequence.

In some aspects, the oligonucleotide, e.g., an ASO of the present disclosure (e.g., ASO1, nusinersen, and analog thereof, a variant thereof, a derivative thereof, or a combination thereof), can function via nuclease mediated degradation of the target nucleic acid, where the oligonucleotides of the disclosure are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example, gapmers.

### IV.D. RNase H Activity and Recruitment

The RNase H activity of an ASO refers to its ability to recruit RNase H when in a duplex with a complementary RNA molecule and induce degradation of the complementary RNA molecule. PCT Publication WO01/23613, which is herein incorporated by reference in its entirety, provides in vitro methods for determining RNaseH activity, which can be used to determine the ability to recruit RNaseH. Typically, an oligonucleotide, e.g., an ASO of the present disclosure (e.g., ASO1, nusinersen, and analog thereof, a variant thereof, a derivative thereof, or a combination thereof), is deemed capable of recruiting RNase H if, when provided with a complementary target nucleic acid sequence, it has an initial rate, as measured in pmol/l/min, of at least 5%, such as at least 10% or more than 20% of the of the initial rate determined when using a oligonucleotide, e.g., an ASO of the present disclosure (e.g., ASO1, nusinersen, and analog thereof, a variant thereof, a derivative thereof, or a combination thereof), having the same base sequence as the modified oligonucleotide being tested, but containing only DNA monomers, with phosphorothioate linkages between all monomers in the oligonucleotide, and using the methodology provided by Example 91 - 95 of PCT Publication WO01/23613.

In some aspects, an oligonucleotide, e.g., an ASO of the present disclosure (e.g., ASO1, nusinersen, and analog thereof, a variant thereof, a derivative thereof, or a combination thereof), is deemed essentially incapable of recruiting RNaseH if, when provided with the complementary target nucleic acid, the RNaseH initial rate, as measured in pmol/l/min, is less than 20%, such as less than 10%, such as less than 5% of the initial rate determined when using a oligonucleotide having the same base sequence as the oligonucleotide being tested, but containing only DNA monomers, with no 2′ substitutions, with phosphorothioate linkages between all monomers in the oligonucleotide, and using the methodology provided by Example 91 - 95 of PCT Publication WO01/23613.

### IV.E. ASO Design

The ASO of the disclosure (e.g., ASO1, nusinersen, and analog thereof, a variant thereof, a derivative thereof, or a combination thereof) can comprise a nucleotide sequence that comprises both nucleosides and nucleoside analogs, and can be in the form of a gapmer. Examples of configurations of a gapmer that can be used with the ASO of the disclosure are described in U.S. Pat. Appl. Publ. No. 2012/0322851.

The term “gapmer” as used herein refers to an antisense oligonucleotide which comprises a region of RNase H recruiting oligonucleotides (gap) which is flanked 5′ and 3′ by one or more affinity enhancing modified nucleosides (flanks). The term “LNA gapmer” is a gapmer oligonucleotide wherein at least one of the affinity enhancing modified nucleosides is an LNA nucleoside. The term “mixed wing gapmer” refers to an LNA gapmer wherein the flank regions comprise at least one LNA nucleoside and at least one DNA nucleoside or non-LNA modified nucleoside, such as at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acid (ANA), and 2′-Fluoro-ANA nucleoside(s).

In some aspects, the ASO of the disclosure (e.g., ASO1, nusinersen, and analog thereof, a variant thereof, a derivative thereof, or a combination thereof) can be in the form of a mixmer. In some aspects, the ASO of the disclosure can be in the form of a totalmer. In some aspects, in addition to enhancing affinity of the ASO for the target region, some nucleoside analogs also mediate RNase (e.g., RNaseH) binding and cleavage. Since α-L-LNA monomers recruit RNaseH activity to a certain extent, in some aspects, gap regions (e.g., region B as referred to herein) of ASOs containing α-L-LNA monomers consist of fewer monomers recognizable and cleavable by the RNaseH, and more flexibility in the mixmer construction is introduced.

### IV.E.1. Gapmer Design

In some aspects, the ASO of the disclosure (e.g., ASO1, nusinersen, and analog thereof, a variant thereof, a derivative thereof, or a combination thereof) is a gapmer and comprises a contiguous stretch of nucleotides (e.g., one or more DNA) which is capable of recruiting an RNase, such as RNaseH, referred to herein in as region B (B), wherein region B is flanked at both 5′ and 3′ by regions of nucleoside analogs 5′ and 3′ to the contiguous stretch of nucleotides of region B- these regions are referred to as regions A (A) and C (C), respectively. In some aspects, the nucleoside analogs are sugar modified nucleosides (e.g., high affinity sugar modified nucleosides). In certain aspects, the sugar-modified nucleosides of regions A and C enhance the affinity of the ASO for the target nucleic acid (i.e., affinity enhancing 2′ sugar modified nucleosides). In some aspects, the sugar-modified nucleosides are 2′ sugar modified nucleosides, such as high affinity 2′ sugar modifications, such as LNA and/or 2′-MOE.

In a gapmer, the 5′ and 3′ most nucleosides of region B are DNA nucleosides, and are positioned adjacent to nucleoside analogs (e.g., high affinity sugar modified nucleosides) of regions A and C, respectively. In some aspects, regions A and C can be further defined by having nucleoside analogs at the end most distant from region B (i.e., at the 5′ end of region A and at the 3′ end of region C).

In some aspects, the ASO of the present disclosure (e.g., ASO1, nusinersen, and analog thereof, a variant thereof, a derivative thereof, or a combination thereof) comprise a nucleotide sequence of formula (5′ to 3′) A-B-C, wherein: (A) (5′ region or a first wing sequence) comprises at least one nucleoside analog (e.g., 3-5 LNA units); (B) comprises at least four consecutive nucleosides (e.g., 4-24 DNA units), which are capable of recruiting RNase (when formed in a duplex with a complementary RNA molecule, such as the mRNA target); and (C) (3′ region or a second wing sequence) comprises at least one nucleoside analog (e.g., 3-5 LNA units).

In some aspects, region A comprises 3-5 nucleoside analogs, such as LNA, region B consists of 6-24 (e.g., 6, 7, 8, 9, 10, 11, 12, 13, or 14) DNA units, and region C consists of 3 or 4 nucleoside analogs, such as LNA. Such designs include (A-B-C) 3-14-3, 3-11-3, 3-12-3, 3-13-3, 4-9-4, 4-10-4, 4-11-4, 4-12-4, and 5-10-5 . In some aspects, the ASO has a design of LLLDₙLLL, LLLLDₙLLLL, or LLLLLDₙLLLLL, wherein the L is a nucleoside analog, the D is DNA, and n can be any integer between 4 and 24. In some aspects, n can be any integer between 6 and 14. In some aspects, n can be any integer between 8 and 12. In some aspects, the ASO has a design of LLLMMDₙMMLLL, LLLMDnMLLL, LLLLMMDnMMLLLL, LLLLMDnMLLLL, LLLLLLMMDnMMLLLLL, or LLLLLLMDnMLLLLL, wherein the D is DNA, n can be any integer between 3 and 15, the L is LNA, and the M is 2′MOE.

Further gapmer designs are disclosed in PCT Publications WO2004/046160, WO 2007/146511, and WO2008/113832, each of which is hereby incorporated by reference in its entirety.

### IV.F. Internucleotide Linkages

The monomers of the ASOs described herein (e.g., ASO1, nusinersen, and analog thereof, a variant thereof, a derivative thereof, or a combination thereof) are coupled together via linkage groups. Suitably, each monomer is linked to the 3′ adjacent monomer via a linkage group.

The person having ordinary skill in the art would understand that, in the context of the present disclosure, the 5′ monomer at the end of an ASO does not comprise a 5′ linkage group, although it can or cannot comprise a 5′ terminal group.

In some aspects, the contiguous nucleotide sequence comprises one or more modified internucleoside linkages. The terms “linkage group” or “internucleoside linkage” are intended to mean a group capable of covalently coupling together two nucleosides. Nonlimiting examples include phosphate groups and phosphorothioate groups.

The nucleosides of the ASO of the disclosure (e.g., ASO1, nusinersen, and analog thereof, a variant thereof, a derivative thereof, or a combination thereof) or contiguous nucleosides sequence thereof are coupled together via linkage groups. Suitably, each nucleoside is linked to the 3′ adjacent nucleoside via a linkage group.

In some aspects, the internucleoside linkage is modified from its normal phosphodiester to one that is more resistant to nuclease attack, such as phosphorothioate, which is cleavable by RNaseH, also allows that route of antisense inhibition in reducing the expression of the target gene. In some aspects, at least 75%, at least 80%, at least 85%, at least 90%, at least 91%, at least 92%, at least 93%, at least 94%, at least 95%, at least 96%, at least 97%, at least 98%, at least 99%, or 100% of internucleoside linkages are modified.

### V. Pharmaceutical Compositions

Provided herein are pharmaceutical compositions comprising compositions of the present disclosure, e.g., ASO1, nusinersen, a nusinersen analog, a nusinersen variant, a nusinersen derivative, or another splicing-modulatory drug in combination with a histone deacetylase inhibitor (e.g., valproic acid, trichostatin A, or combination thereof; e.g., at a subclinical dose), and a pharmaceutically acceptable carrier or excipient, in a form suitable for administration to a subject, e.g., a subject having SMA.

Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington’s Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition of the present disclosure comprises one or more components of a combination treatment disclosed herein, e.g., in some aspects a pharmaceutical composition comprises ASO1, nusinersen, a nusinersen analog, a nusinersen variant, a nusinersen derivative, or another splicing-modulatory drug, and a second pharmaceutical composition comprises a histone deacetylase inhibitor (e.g., valproic acid, trichostatin A, or combination thereof; e.g., at a subclinical dose). In certain aspects, when the compositions of the present disclosure are in two separate pharmaceutical compositions, such pharmaceutical compositions can be administered concurrently and/or consecutively. In some aspects, the pharmaceutical compositions of the present disclosure are co-administered with one or more additional therapeutic agents (e.g., a methylation inhibitor such as 5-azacytidine) in a pharmaceutically acceptable carrier.

In some aspects, the composition of the present disclosure, e.g., ASO1, nusinersen, a nusinersen analog, a nusinersen variant, a nusinersen derivative, or another splicing-modulatory drug in combination with a histone deacetylase inhibitor (e.g., valproic acid, trichostatin A, or combination thereof; e.g., at a subclinical dose), and the one or more additional therapeutic agents (e.g., a methylation inhibitor such as 5-azacytidine) can be administered in the same pharmaceutical composition.

In some aspects, the pharmaceutical composition disclosure, e.g., a composition comprising ASO1, nusinersen, a nusinersen analog, a nusinersen variant, a nusinersen derivative, or another splicing-modulatory drug in combination with a histone deacetylase inhibitor (e.g., valproic acid, trichostatin A, or combination thereof; e.g., at a subclinical dose) is administered prior to the administration of the additional therapeutic agent(s) (e.g., a methylation inhibitor such as 5-azacytidine).

In some aspects, the pharmaceutical composition of the disclosure, e.g., a composition comprising ASO1, nusinersen, a nusinersen analog, a nusinersen variant, a nusinersen derivative, or another splicing-modulatory drug in combination with a histone deacetylase inhibitor (e.g., valproic acid, trichostatin A, or combination thereof; e.g., at a subclinical dose) is administered after the administration of the additional therapeutic agent(s) (e.g., a methylation inhibitor such as 5-azacytidine).

In some aspects, the pharmaceutical composition of the disclosure, e.g., a composition comprising ASO1, nusinersen, a nusinersen analog, a nusinersen variant, a nusinersen derivative, or another splicing-modulatory drug in combination with a histone deacetylase inhibitor (e.g., valproic acid, trichostatin A, or combination thereof; e.g., at a subclinical dose) is administered after the administration of the additional therapeutic agent(s) (e.g., a methylation inhibitor such as 5-azacytidine).

In further aspects, the pharmaceutical composition of the disclosure, e.g., a composition comprising ASO1, nusinersen, a nusinersen analog, a nusinersen variant, a nusinersen derivative, or another splicing-modulatory drug in combination with a histone deacetylase inhibitor (e.g., valproic acid, trichostatin A, or combination thereof; e.g., at a subclinical dose) is administered concurrently with the additional therapeutic agent(s) (e.g., a methylation inhibitor such as 5-azacytidine).

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer’s solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration.

The pharmaceutical compositions of the disclosure, e.g., a first pharmaceutical composition comprising ASO1, nusinersen, a nusinersen analog, a nusinersen variant, a nusinersen derivative, or another splicing-modulatory drug, and a second pharmaceutical composition comprising a histone deacetylase inhibitor (e.g., valproic acid, trichostatin A, or combination thereof; e.g., at a subclinical dose) can be administered concurrently, or sequentially. In some aspects, the first pharmaceutical composition (e.g., a composition comprising ASO1, nusinersen, a nusinersen analog, a nusinersen variant, a nusinersen derivative, another splicing-modulatory drug, or a combination thereof) is administered prior to the administration of the second pharmaceutical composition (e.g., a composition comprising a histone deacetylase inhibitor such as valproic acid, trichostatin A, or combination thereof; e.g., at a subclinical dose). In other aspects, the first pharmaceutical composition is administered after the administration of the second pharmaceutical composition.

In some aspects, a pharmaceutical composition disclosed herein is administered by intrathecal, parenteral, topical, intravenous, oral, subcutaneous, intra-arterial, intradermal, transdermal, rectal, intracranial, intraperitoneal, intranasal, intratumoral, intramuscular route or as inhalants. In some aspects, the first pharmaceutical composition (e.g., a composition comprising ASO1, nusinersen, a nusinersen analog, a nusinersen variant, a nusinersen derivative, another splicing-modulatory drug, or a combination thereof) is administered by intrathecal, parenteral, topical, intravenous, oral, subcutaneous, intra-arterial, intradermal, transdermal, rectal, intracranial, intraperitoneal, intranasal, intratumoral, intramuscular route or as an inhalant. In some aspects, the second pharmaceutical composition (e.g., a composition comprising a histone deacetylase inhibitor such as valproic acid, trichostatin A, or combination thereof; e.g., at a subclinical dose) is administered by intrathecal, parenteral, topical, intravenous, oral, subcutaneous, intra-arterial, intradermal, transdermal, rectal, intracranial, intraperitoneal, intranasal, intratumoral, intramuscular route or as an inhalant.

In some aspects, the first pharmaceutical composition (e.g., a composition comprising ASO1, nusinersen, a nusinersen analog, a nusinersen variant, a nusinersen derivative, another splicing-modulatory drug, or a combination thereof) is administered by intrathecal or intravenous route. In some aspects, the second pharmaceutical composition (e.g., a composition comprising a histone deacetylase inhibitor such as valproic acid, trichostatin A, or combination thereof; e.g., at a subclinical dose) is administered by intrathecal, parenteral, intravenous, oral, or intraperitoneal route. In some aspects, the first pharmaceutical composition (e.g., a composition comprising ASO1, nusinersen, a nusinersen analog, a nusinersen variant, a nusinersen derivative, another splicing-modulatory drug, or a combination thereof) is administered by intrathecal or intravenous route, and the second pharmaceutical composition (e.g., a composition comprising a histone deacetylase inhibitor such as valproic acid, trichostatin A, or combination thereof; e.g., at a subclinical dose) is administered by intrathecal, parenteral, intravenous, oral, or intraperitoneal route.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if the therapeutic agent is water-soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound that delays absorption, e.g., aluminum monostearate and gelatin.

Sterile injectable solutions can be prepared by incorporating a composition of the present disclosure in an effective amount and in an appropriate solvent with one or more ingredients enumerated herein or known in the art, as desired. Generally, dispersions are prepared by incorporating a composition of the present disclosure into a sterile vehicle that contains a basic dispersion medium and any desired other ingredients. In the case of sterile powders for the preparation of sterile injectable solutions, methods of preparation are vacuum drying and freeze-drying that yield a powder of the active ingredient plus any additional desired ingredient from a previously sterile-filtered solution thereof.

The pharmaceutical compositions the present disclosure (e.g., a first pharmaceutical composition comprising ASO1, nusinersen, a nusinersen analog, a nusinersen variant, a nusinersen derivative, another splicing-modulatory drug, or a combination thereof; a second pharmaceutical composition, e.g., a composition comprising a histone deacetylase inhibitor such as valproic acid, trichostatin A, or combination thereof; e.g., at a subclinical dose; or a composition comprising the active principles of both the first and second pharmaceutical compositions) can be administered in the form of a depot injection or implant preparation which can be formulated in such a manner to permit a sustained or pulsatile release of the active principles (e.g., ASO1, nusinersen, a nusinersen analog, a nusinersen variant, a nusinersen derivative, another splicing-modulatory drug, a histone deacetylase inhibitor such as valproic acid or trichostatin A, or any combination thereof; e.g., at a subclinical dose).

Systemic administration of pharmaceutical compositions the present disclosure can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives.

In certain aspects, a pharmaceutical composition the present disclosure (e.g., a first pharmaceutical composition comprising ASO1, nusinersen, a nusinersen analog, a nusinersen variant, a nusinersen derivative, another splicing-modulatory drug, or a combination thereof; a second pharmaceutical composition, e.g., a composition comprising a histone deacetylase inhibitor such as valproic acid, trichostatin A, or combination thereof; e.g., at a subclinical dose; or a composition comprising the active principles of both the first and second pharmaceutical compositions) can be administered intravenously into a subject that would benefit from the pharmaceutical composition.

In certain aspects, a pharmaceutical composition of the present disclosure can be administered intrathecally into a subject that would benefit from the pharmaceutical composition. In certain aspects, a pharmaceutical composition of the present disclosure can be administered perineurally into a subject that would benefit from the pharmaceutical composition. In certain aspects, a pharmaceutical composition of the present disclosure can be administered intraneurally into a subject that would benefit from the pharmaceutical composition. In some aspects, a pharmaceutical composition of the present disclosure can be administered as a liquid suspension.

In certain aspects, a pharmaceutical composition the present disclosure (e.g., a first pharmaceutical composition comprising ASO1, nusinersen, a nusinersen analog, a nusinersen variant, a nusinersen derivative, another splicing-modulatory drug, or a combination thereof; a second pharmaceutical composition (e.g., a composition comprising a histone deacetylase inhibitor such as valproic acid, trichostatin A, or combination thereof; e.g., at a subclinical dose; or a composition comprising the active principles of both the first and second pharmaceutical compositions) can be administered as a formulation that is capable of forming a depot following administration. In certain aspects, the depot slowly releases the pharmaceutical composition the present disclosure (e.g., a first pharmaceutical composition comprising ASO1, nusinersen, a nusinersen analog, a nusinersen variant, a nusinersen derivative, another splicing-modulatory drug, or a combination thereof; a second pharmaceutical composition (e.g., a composition comprising a histone deacetylase inhibitor such as valproic acid, trichostatin A, or combination thereof; e.g., at a subclinical dose; or a composition comprising the active principles of both the first and second pharmaceutical compositions) into circulation, or remains in depot form.

Typically, pharmaceutically acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically acceptable carrier can be lactose, dextrose, sucrose, sorbitol, mannitol, starch, gum acacia, calcium phosphate, alginates, gelatin, calcium silicate, microcrystalline cellulose, polyvinylpyrrolidone, cellulose, water, syrup, methylcellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

In some aspects, the pharmaceutical compositions described herein comprise a pharmaceutically acceptable salt. In some aspects, the pharmaceutically acceptable salt comprises a sodium salt, a potassium salt, an ammonium salt, or any combination thereof. In some aspects, a composition of the present disclosure comprises a pharmaceutically acceptable salt of an ASO such as ASO1, nusinersen, a nusinersen analog, a nusinersen variant, a nusinersen derivative, or a combination thereof. In some aspects, a composition of the present disclosure comprises a pharmaceutically acceptable salt of a histone deacetylase inhibitor such as valproic acid.

The pharmaceutical compositions described herein comprise optionally an additional pharmaceutically active or therapeutic agent. The additional therapeutic agent can be, for example, a biological agent, a small molecule agent, or a nucleic acid agent. In some aspects, the additional therapeutic agent is an additional splicing-modulatory drug, e.g., an ASO, such as an ASO targeting SMN2 (e.g., an ASO targeting SMN2 E7). In some aspects, the splicing-modulatory is any ASO targeting SMN2 (e.g., an ASO targeting SMN2 E7) disclosed herein or a combination thereof. In some aspects, the additional therapeutic agent is a small molecule, e.g., a histone deacetylase inhibitor a methylation inhibitor, or a combination thereof. In some aspects, the additional therapeutic agent is a chemical compound, an siRNA, an shRNA, an antisense oligonucleotide, a protein, or any combination thereof. In some aspects, the additional therapeutic agent is an agent useful for the treatment of SMA.

Dosage forms are provided that comprise a pharmaceutical composition comprising a composition of the present disclosure. In some aspects, the dosage form is formulated as a liquid suspension for intravenous injection. In some aspects, the dosage form is formulated as a liquid suspension for intrathecal administration, e.g., intrathecal injection. In some aspects, the dosage form is formulated for oral administration.

### VI. Kits and Products of Manufacture

Also provided herein are kits and products of manufacture comprising one or more compositions (e.g., pharmaceutical compositions) described herein. In some aspects, provided herein is a pharmaceutical pack or kit comprising one or more containers filled with one or more of the ingredients of the pharmaceutical compositions described herein.

In some aspects, the kit or product of manufacture comprises, e.g., a first container comprising a first pharmaceutical composition comprising ASO1, nusinersen, a nusinersen analog, a nusinersen variant, a nusinersen derivative, another splicing-modulatory drug, or a combination thereof; and a second container comprising a second pharmaceutical composition comprising a histone deacetylase inhibitor such as valproic acid, trichostatin A, or combination thereof (e.g., at a subclinical dose), and optionally an instruction for use.

In some aspects, the kit or product of manufacture comprises a container comprising ASO1, nusinersen, a nusinersen analog, a nusinersen variant, a nusinersen derivative, another splicing-modulatory drug, or a combination thereof; and a histone deacetylase inhibitor such as valproic acid, trichostatin A, or combination thereof (e.g., at a subclinical dose), and optionally an instruction for use.

In some aspects, the kit contains a pharmaceutical composition described herein and any prophylactic or therapeutic agent, such as those described herein. In some aspects, the kit further comprises instructions to administer a composition of the present disclosure according to any method disclosed herein. In some aspects, the kit is for use in the treatment of a SMA. In some aspects, the kit is a diagnostic kit.

All of the references cited above, as well as all references cited herein, are incorporated herein by reference in their entireties.

The sequences of biomolecules (e.g., proteins, genes, miRNAs, SNPs) disclosed herein and identified by either database accession number or gene name are incorporated by reference. The database accession numbers disclosed herein (e.g, Genbak accession numbers) refer to the database version that in effect on Jul. 13, 2020. The nucleic acid sequences of genes identified by name as well as their official names and alternative names correspond to those in the version of the Genbank database active on Jul. 13, 2020, and are herein incorporated by reference. The amino acid sequences of proteins identified by name or translation products of genes identified by name as well as their official and alternative names correspond to those in the version of the UniProt database active on Jul. 13, 2020, and are herein incorporated by reference.

The following examples are offered by way of illustration and not by way of limitation.

### EXAMPLES

### Experimental Procedures

### Antisense Oligonucleotide Synthesis

ASO1 (5′-ATTCACTTTCATAATGCTGG-3′) (SEQ ID NO:1), a seven-mismatch control (5′-AATCATTTGCTTCATACAGG-3′) and ASO2 (5′-AAAGTATGTTTCTTCCACAC-3′) (SEQ ID NO:2) 2′-O-methoxyethyl-modified oligonucleotides with phosphorothioate backbone and all 5-methyl cytosines were purchased from IDT, and a 2′-OMe-modified phosphorothioate oligonucleotide (5′-AUUCACUUUCAUAAUGCUGG-3′) (SEQ ID NO:3) (Singh et al., 2006) was purchased from TriLink. The oligonucleotides were dissolved in 0.9% w/v saline.

### RNAPII Expression Vectors and Alternative Splicing Reporter Minigene

The expression vectors for α-amanitin-resistant variants of the large subunit of human RNAPII (Rpb1) wild-type (WTres; pAT7Rpb1αAmr vector), and the hC4 mutant (pAT7Rpb1αAmrR749H) were previously described (de la Mata et al., Molecular Cell, 12, 525-53, 2003). An α-amanitin-sensitive Rpb1 expression vector (WTs) was used as a control. The pCI-SMN2 (Addgene, Plasmid #72287) minigene vector for SMN2 was described previously (Lorson et al,. Proc. Natl. Acad. Sci. 96, 6307-6311, 1999).

### Cell Culture and Treatments

HEK293T, HeLa, and SMA type I homozygous and carrier fibroblasts 3813 and 3814 (Coriell Cell Repositories, Camden, New Jersey, United States) were grown in Dulbecco’s modified Eagle’s medium (DMEM) containing 4.5 g of glucose and 10% fetal bovine serum (Gibco) at 37° C. Cells were plated at a density of 2×10⁵ cells per well in 12-well plates 24 hr before transfection. siRNA (25 nM), plasmid (500 ng), or ASO (25 nM) transfections were performed 24 hr after cells were plated, using 3 µl of Lipofectamine 2000 (Thermo Fisher Scientific) per well in 12-well plates. 24-48 h later, cells were treated with Trichostatin A (Sigma, T8552), Valproic Acid (Sigma, P4543), 5-Azacytidine (Sigma, A2385) or vehicle for the indicated time, and harvested for downstream procedures.

### RNA Extraction and RT-PCR

Twenty milligrams of mouse tissue were pulverized in liquid N₂ with mortar and pestle, and homogenized with 1 mL of Trizol (Invitrogen). Total RNA was isolated according to the manufacturer’s directions. One microgram of RNA was reverse-transcribed with M-MLV reverse transcriptase (Invitrogen) and oligo-dT primer, and the cDNA was amplified. Amplification and analysis of SMN2 transcripts was performed as described (Hua et al., Am. J. Hum. Genet. 82, 834-848, 2008).

### Western Blot

Twenty milligrams of tissue were pulverized in liquid N₂ and homogenized in 0.4 mL (liver, kidney, muscle, spinal cord, brain, and heart) of 1× protein sample buffer containing 2% (w/v) SDS, 10% (v/v) glycerol, 50 mM Tris-HCl (pH 6.8), and 0.1 M DTT. Protein samples were separated by 12% SDS-PAGE and electroblotted onto nitrocellulose membranes. The blots were probed with mAb anti-hSMN (BD Biosciences, 610646), or pAb anti-β-tubulin (Sigma), followed by secondary IRDye 800CW-conjugated goat anti-mouse or anti-rabbit antibody. Protein signals were detected with an Odyssey instrument (LI-COR Biosciences).

### RNAi Knockdown

Downregulation of hnRNP A1 and A2 was performed using ON-TARGET plus SMARTpool siRNA oligonucleotides (Dharmacon). siRNA oligos were delivered to cells following the manufacturer’s instructions, and allowed to act for 72 hr. Accell siRNA antihuman non targeting siRNA (Dharmacon, NC1567415) was used as a control.

### Chromatin Immunoprecipitation (xChIP)

Approximately 2×10⁶ HEK293T cells per sample were treated for 10 min in 1% (v/v) formaldehyde at room temperature to crosslink protein-DNA complexes. Crosslinking was stopped with glycine at a final concentration of 125 mM. Cells were washed twice with cold PBS and swelled on ice for 10 min in 25 mM HEPES pH 8, 1.5 mM MgCl₂, 10 mM KCl, 0.1% NP-40, 1 mM DTT and 1× protease inhibitor cocktail set III (Calbiochem). Following Dounce homogenization, the nuclei were collected and resuspended in 1 ml sonication buffer (50 mM HEPES pH 8, 140 mM NaCl, 1 mM EDTA, 0.1% sodium deoxycholate, 0.1% SDS and 1× protease inhibitor cocktail). DNA was sonicated in an ultrasonic bath (Bioruptor Diagenode) to an average length of 200-500 bp. After addition of 1% (v/v) Triton X-100, samples were centrifuged at 15,000 ×g. Supernatants were immunoprecipitated O/N with 40 µl of pre-coated anti-IgG magnetic beads (Dynabeads M-280, Invitrogen) previously incubated with the antibody of interest for 6 hr at 4° C. The antibodies used were: rabbit anti-H3 (2 µg, Abcam ab1791), mouse anti-H3K9me2 (4 µg, Abcam ab1220), rabbit anti H3K9me3 (4 µg, Abcam ab8898), rabbit anti H3K9Ac (2 µg, Abcam ab4441), rabbit anti Rpb1 NTD (2 µg, Cell Signaling, D8L4Y), rabbit Phospho-Rpb1 CTD Ser2 (2 µg, Cell Signaling, E1Z3G), rabbit Phospho-Rpb1 CTD Ser5 (2 µg, Cell Signaling, D9N5I). Control immunoprecipitations were performed with rabbit IgG (1 µg, Abcam ab171870). Beads were washed sequentially for 5 min each in Low-salt (20 mM Tris-HCl pH 8, 150 mM NaCl, 2 mM EDTA, 1% Triton X-100, 0.1% SDS), High-Salt (20 mM Tris-HCl pH 8, 500 mM NaCl, 2 mM EDTA, 1% Triton X-100, 0.1% SDS) and LiCl buffer (10 mM Tris pH 8.0, 1 mM EDTA, 250 mM LiCl, 1% NP-40, 1% Na-deoxycholate) for 5 min at 4° C. and then twice in TE 1x for 2 min at room temperature. Beads were eluted in 1% SDS and 100 mM NaHCO₃ buffer for 15 min at 65° C. and crosslinking was reversed for 6 hr after addition of NaCl to a final concentration of 200 mM. Chromatin was precipitated with ethanol overnight, treated with 20 µg proteinase K, and purified by phenol-chloroform extraction. Immunoprecipitated DNA (1.5 µl) and serial dilutions of the 10% input DNA (1:4, 1:20, 1: 100, and 1:500) were analyzed by SYBR-Green real-time qPCR. The oligonucleotide sequences used are listed in the table below.

TABLE 1

Oligonucleotide primers

| Target | Forward | SEQ ID NO | Reverse | SEQ ID NO |
|---|---|---|---|---|
| SMN2 mRNA | AAGTGAGAACTCCAGGTCTCCTG | 4 | TCTGATCGTTTCTTTAGTGGTGTC | 5 |
| SMN2 pre mRNA | CGGGTTTGCTATGGCGATGAG | 6 | GGCTGCGGAAGGAGAGTTGG | 7 |
| SMN2 minigene | CCTTCCATATTCCAGTTCTCTTG | 8 | TACCTGTAACGCTTCACATTCC | 9 |
| SMN_36 | CAAATGTGGGAGGGCGATAACC | 10 | TTCTGGGAGCGGAACAGTACG | 11 |
| SMN_38 | GGCTCACTACAACCTCCTC | 12 | TTCAGATTATTCTCCTCCATTCC | 13 |
| SMN_51 | TCCTTACAGGGTTTTAGACAAAATC | 14 | CATAATGCTGGCAGACTTACTCC | 15 |
| SMN_50 | GCTCAGGTGATCCAACTGTCTC | 85 | CGTGGTGGCTCAGGCTAGG | 86 |
| SMN_138 | ACATATAAGCCATTTAGCAACCC | 16 | GAACTCCTGACCTCGTAATCC | 17 |
| SMN_139 | GAGACCAGCCTACACAATATGC | 18 | CCGCCTCAACCTCCCAAG | 19 |
| SMN_140 | CCGAAACCCCGTCTCTACTAAATAC | 20 | AAGCGATCCTCCCACCTCAG | 21 |
| SMN_141 | AATGTAGTATGGTTCTGTGTCTC | 22 | GTTTGCTCAAGGTAGTCTGG | 23 |
| SMN_42 | TTTATCTCCCTCCCGCTATTC | 24 | AATCTCTTTGAGTCTTAGTTTCCC | 25 |
| SMN_46 | TCTCTTGATGATGCTGATGC | 26 | AGATGCTGAGTGATTACTTACC | 27 |
| SMN_135 | GCAAGGCAAGGCATTACAG | 28 | CCTGAGCAATAGAGTGAGACC | 29 |
| SMN_136 | CACTGTGCCTAGCCTGAG | 30 | GGATCACTTGAGACCTGGAG | 31 |
| SMN_93 | ACTGGTTGGTTGTGTGGAAG | 32 | AGTCTGCTGGTCTGCCTAC | 33 |
| MYOD_1 | ACAGTGGGTGGGCATTCAG | 34 | GAGCGGTGGCGACAGTAG | 35 |
| MYOD_2 | TCTCTGCCGCTTGGGTTG | 36 | CATAGAAGTCGTCCGTTGTGG | 37 |
| GAPDH_1 | CTCTCTCCCATCCCTTCTCC | 38 | TTGCCAAGTTGCCTGTCC | 39 |
| GAPDH_2 | ATGAATGGGCAGCCGTTAG | 40 | TCGCTCCACCTGACTTCC | 41 |
| hnRNPA1_1 | GCATCGTTAAAGTCTCTCTTCACC | 42 | CTCCTCAGGCTCTCATCAGTTG | 43 |
| hnRNPA1_2 | TGGCATTAAAGAAGACACTGAAG | 44 | TCAAAGGTTACAAAGGCAAAGC | 45 |
| SMN2 transgene | AAGTGAGAACTCCAGGTCTCCTG | 46 | CTTCTGAGTCTTGGGCATGTCAGTG | 47 |
| Gapdh | GAATGGGAAGCTTGTCATCAACGG | 48 | CCGTTCAGCTCTGGGATGACCTTG | 49 |
| Kdm5d | CTGAAGCTTTTGGCTTTGAG | 50 | CCGCTGCCAAATTCTTTGG | 51 |
| Sry | TTGTCTAGAGAGCATGGAGGGCCATGTCAA | 52 | CCACTCCTCTGTGACACTTTAGCCCTCCGA | 53 |
| S1 | ATAACACCACCACTCTTACTC | 54 | | |
| S2 | GTAGCCGTGATGCCATTGTCA | 55 | | |
| H1 | | | AGCCTGAAGAACGAGATCAGC | 56 |

### Animals

All mouse protocols were in accordance with Cold Spring Harbor Laboratory’s Institutional Animal Care and Use Committee guidelines.

All mouse protocols were in accordance with Cold Spring Harbor Laboratory’s Institutional Animal Care and Use Committee guidelines. The mild Hung mouse model (Smn⁻^{/-}; SMN2^{2TG/2TG}) was the strain FVB.Cg-Smn1^{tm1Hung} Tg(SMN2)2^{Hung/J}, purchased from Jackson Laboratory (stock no. 005058). The severe SMA model (Smn^{-/-}; SMN2^{2TG/0}) was generated as previously described (Hua et al., Nature 478, 123-126, 2011).

### Genotyping

For each animal, the genotype was verified by previously described genotyping PCR reactions using tail-tip DNA. Primer sequences and PCR conditions were previously described (Hsieh-Li et al., Nat. Genet. 24, 66-70, 2000).

### Administration of Oligonucleotides to hSMN2 Transgenic Mice

Oligonucleotide solutions in saline were injected subcutaneously into the upper back at P0 and P1 with a 5-mL syringe and 33-gauge custom removable needle (Hamilton) as described (Hua et al., Nature 478, 123-126, 2011). All drugs were injected subcutaneously before P3, contralateral to the oligonucleotide-injection site, with TSA (10 mg/kg) or VPA (10 mg/kg) or vehicle. Mouse tissues and organs, including liver, thigh muscles, kidney, and spinal cord, were snap-frozen in liquid N2 and stored at -70° C.

### Example 1

### Slow Elongation Inhibits SMN2 E7 Inclusion

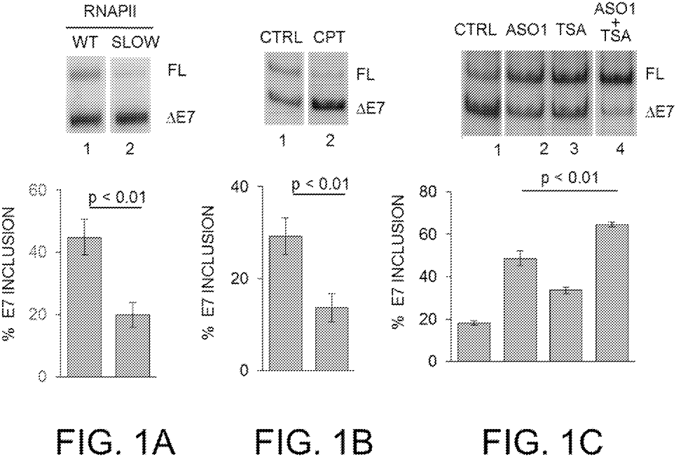

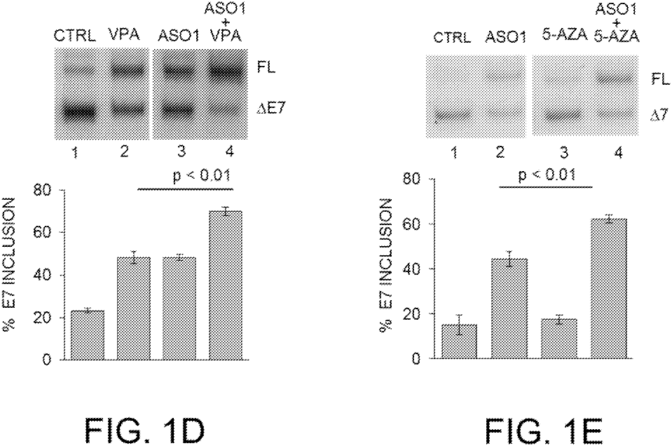

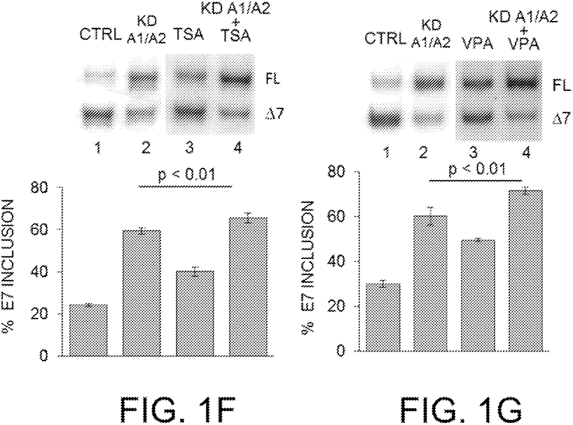

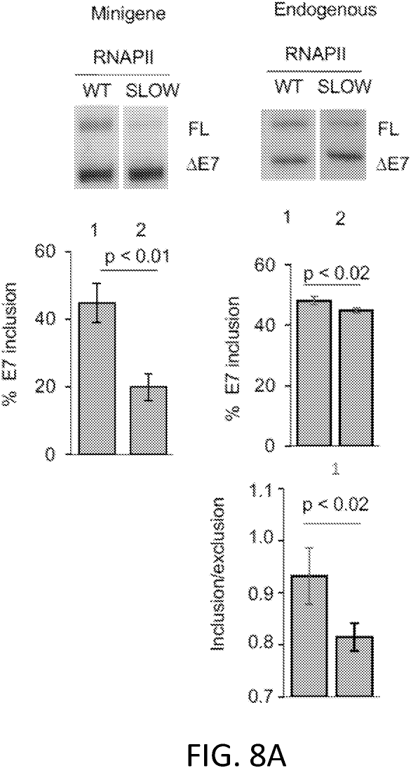

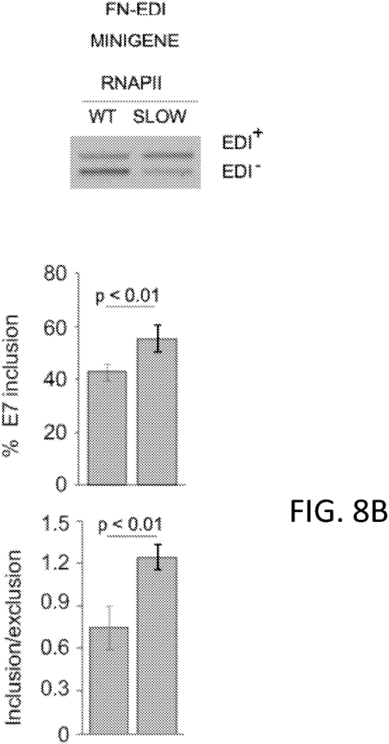

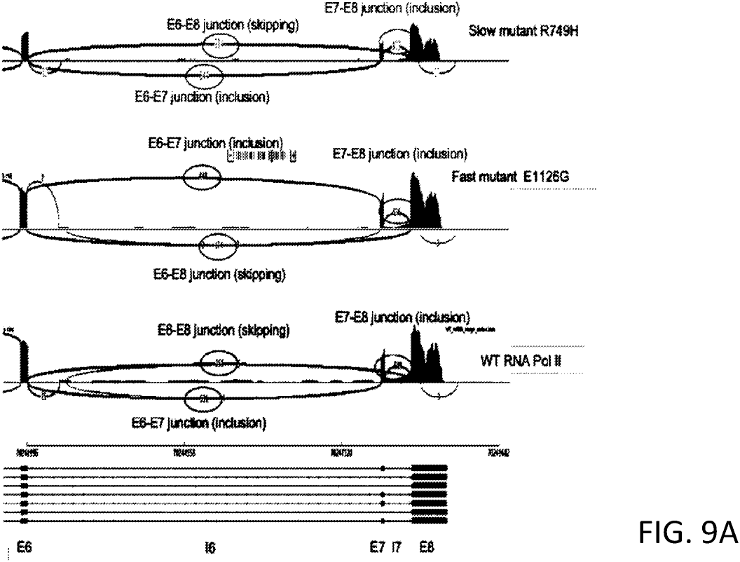

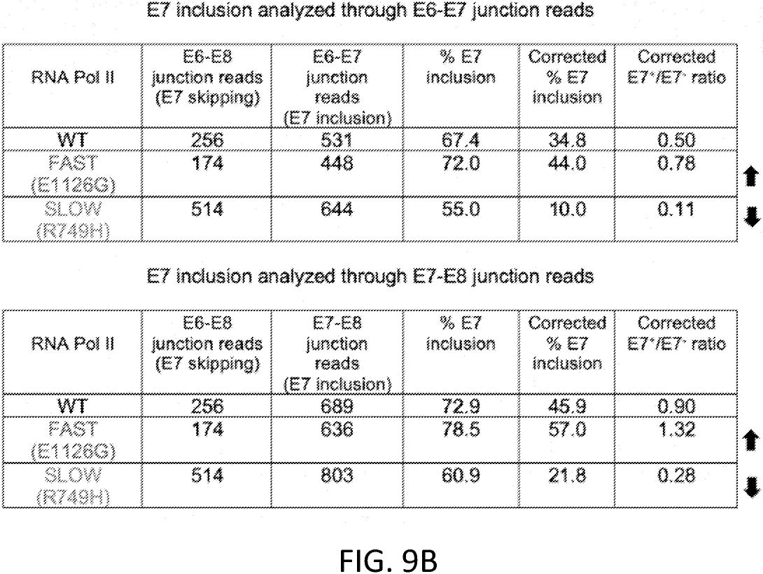

To evaluate the impact of transcription elongation rate on SMN2 E7 inclusion, the effects of a slow mutant of RNAPII were assessed. HEK293T cells were co-transfected with an SMN2 E7 reporter minigene (Lorson et al., Proc. Natl. Acad. Sci. USA 96, 6307-6311, 1999) and a plasmid encoding an alpha-amanitin-resistant large subunit of RNAPII, with or without the point mutation R749H (de la Mata et al., Mol. Cell 12, 525-53, 2003), previously shown to slow elongation in vivo by 2- to 3-fold (Boireau et al., J. Cell Biol. 179, 291-304, 2007). When transcription was carried out by the slow polymerase, SMN2 E7 skipping was greater than upon transcription by the alpha-amanitin-resistant wild-type polymerase (FIG. 1A). The effects of the slow polymerase were also observed on the endogenous SMN2 gene (FIGS. 8A-8B). Furthermore, reanalysis of published genome-wide sequencing data confirmed not only that the slow polymerase causes E7 skipping, but that a fast mutant (E1126G) has the expected opposite effect, promoting E7 inclusion (FIGS. 9A-9B)

Consistently, treatment of cells with the DNA topoisomerase I inhibitor camptothecin (CPT), which indirectly inhibits elongation (Dujardin et al., Mol. Cell 54, 683-690, 2014; Listerman et al., Nat. Struct. Mol. Biol. 13, 815-822, 2006), also promoted E7 skipping, in this case assessed on transcripts from the endogenous SMN2 gene (FIG. 1B). These results indicated that SMN2 E7 is a class II elongation-sensitive exon.

### Example 2

### Cooperative Effects of ASO1 and HDAC Inhibitors in Cells in Culture

Slow polymerase and CPT causes E7 skipping. Thus, whether chromatin opening by histone acetylation foster E7 inclusion by promoting RNAPII elongation was evaluated. The effects of HDAC inhibitors, such as trichostatin A (TSA), on E7 inclusion in HEK293T cells were measured, either alone or in combination with transfection of ASO1 (MOE), a 2′-O-(2-methoxyethyl) phosphorothioate-modified antisense oligonucleotide, at suboptimal concentrations. 2′-OMe-modified phosphorothioate oligonucleotide (OMe) with the same sequence as ASO1 gave the same results as MOE in these transfection experiments (data not shown).

Nusinersen was originally dubbed ASO¹⁰⁻²⁷. The ASO1 variant, which has a 2-nucleotide extension and can be described as ASO¹⁰⁻²⁹, is equally effective as a treatment of SMA The extension of the ASO1 by two nucleotides can have beneficial effects, e.g., increasing the strength of the interaction and the specificity of the interaction with the target area by providing additional contact points to stabilitize the ASO/target complex, which could favor the specific binding of the ASO to the target site and reduce toxicities caused by off-site interactions. See, e.g., U.S. Pat. No. 9,879,265, which discloses a 16-mer ASO capable of inhibiting PCSK9 without causing any kidney toxicity, whereas a corresponding ASO two nucleotides shorter disclosed in WO 2011/009697 and in Poelgeest et al. (Am J Kidney Dis. 62(4): 796-800, 2013, was shown to cause acute kidney toxicity.

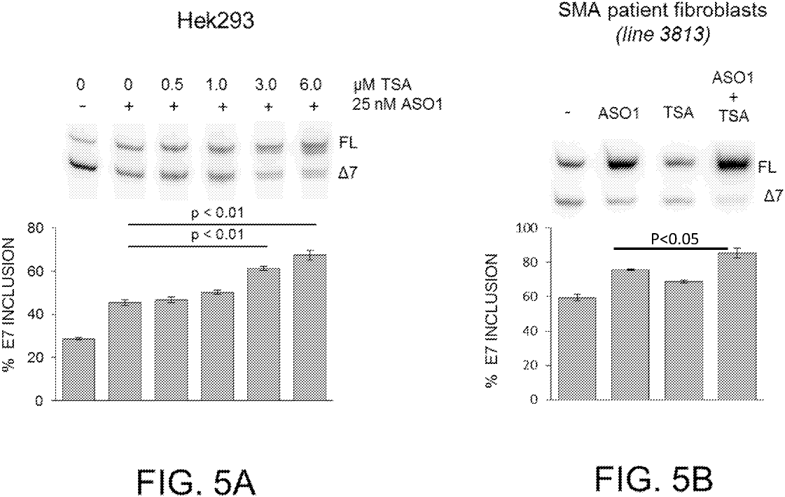

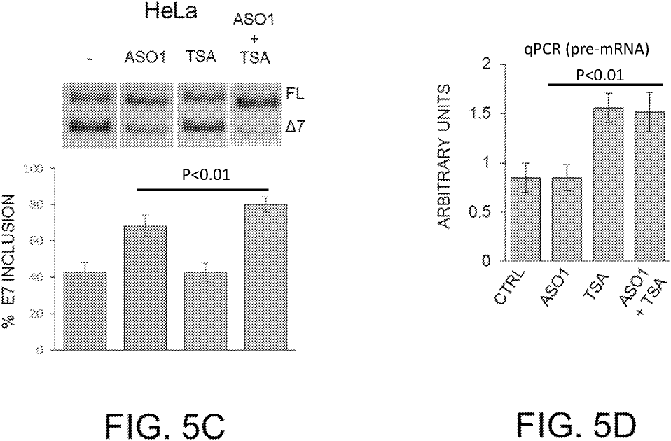

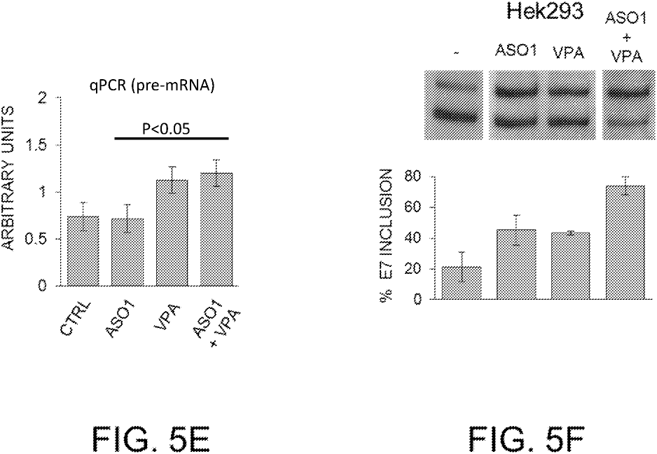

The data presented in FIG. 1C showed that TSA alone was less potent than ASO1 in upregulating E7 inclusion, but combining both drugs greatly enhanced the effect. The cooperative effect of TSA was dose-dependent (FIG. 5A) and reproducible in other cell types, such as HeLa cells (FIG. 5B) and SMA patient fibroblasts (FIG. 5C). Similar results were obtained by combining ASO1 and VPA, a HDAC inhibitor that is approved for clinical use (Wirth et al., Semin. Pediatr. Neurol. 13, 121-131, 2006) (FIG. 1D).

H3K9 methylation is a transcriptionally repressive mark that competes with the transcriptionally permissive H3K9 acetylation (Ghare et al., Immunol. 1, 412-421, 2014). Hence, inhibition of methylation should mimic the effects of increased acetylation. This was confirmed when cells were treated with 5-aza cytidine (5-AZA), a drug that inhibits both DNA methylation and histone methylation (Wozniak et al., Oncogene 26, 77-90, 2007). Though 5-AZA had no effect per se on E7 inclusion, it enhanced the effects of ASO1 as effectively as the HDAC inhibitors (FIG. 1E). Finally, in agreement with the fact that ASO1 displaces the splicing repressors hnRNPA1/A2 from their pre-mRNA target site in intron 7, we found a synergistic effect between HDAC inhibition and downregulation of hnRNPA1/A2 levels by RNAi, in the absence of ASO1. Upon treatment with either TSA (FIG. 1F) or VPA (FIG. 1G), the effects of hnRNPA1/A2 depletion in promoting E7 inclusion were stronger.

VPA was previously assessed in the context of SMA (Swoboda et al., PLoS One 5, e12140, 2010), but not in combination with any ASO, e.g., nusinersen. Clinical trials with VPA (plus carnitine) failed to demonstrate effectiveness (Kissel et al., PLoS One 6, e21296, 2011). The rationale for the clinical use of VPA was that chromatin opening at the SMN2 promoter would increase its transcription and therefore SMN levels (Kernochan et al., Hum. Mol. Genet. 14, 1171-1182, 2005). Both TSA and VPA indeed increased the levels of SMN2 pre-mRNA in in HEK293T cells, measured with a promoter-proximal amplicon as a proxy to transcription levels (FIG. 5D and FIG. 5E). This effect of the HDAC inhibitors alone was not observed in mice.

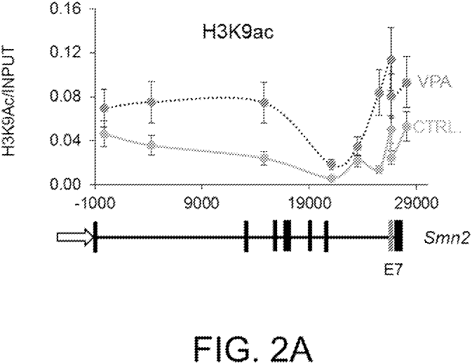

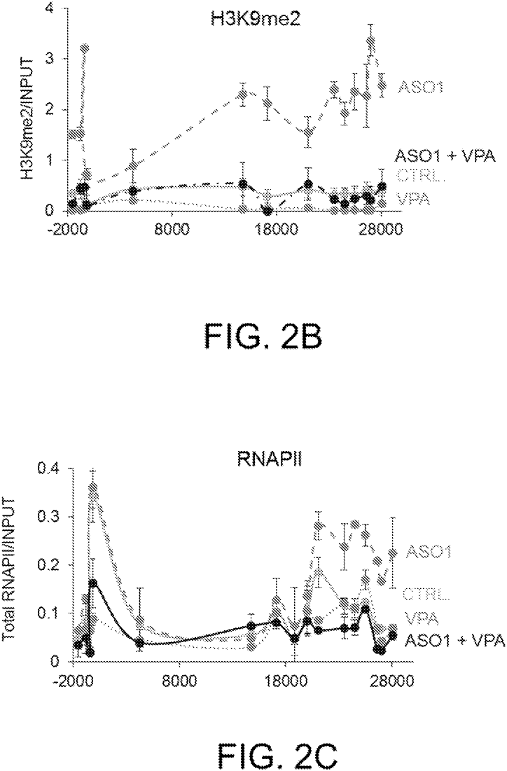

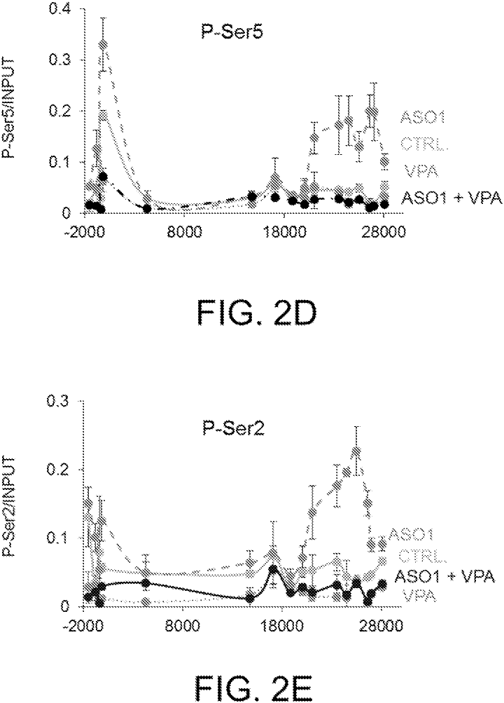

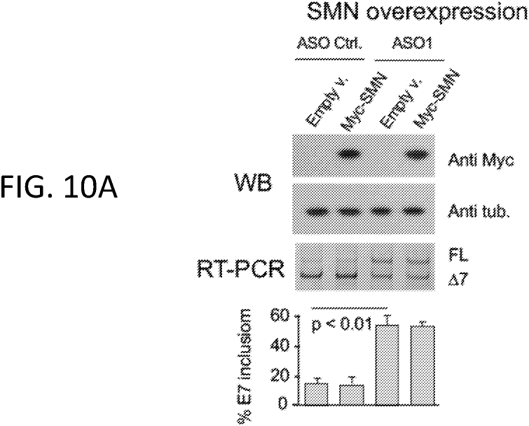

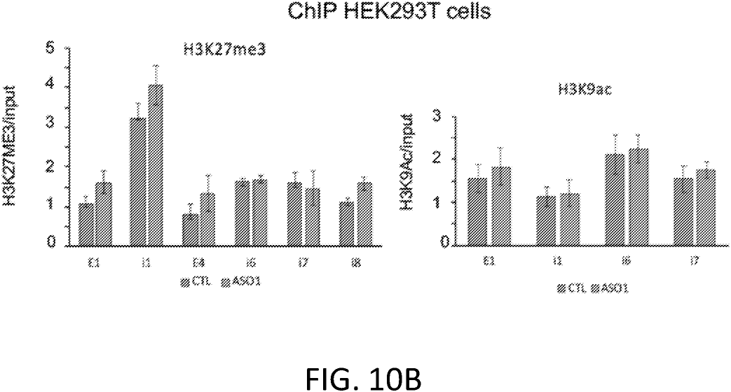

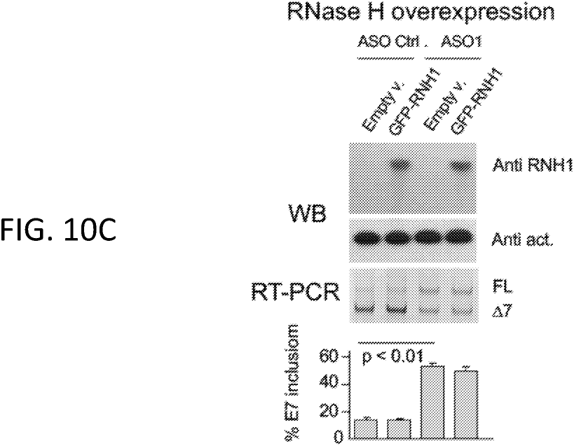

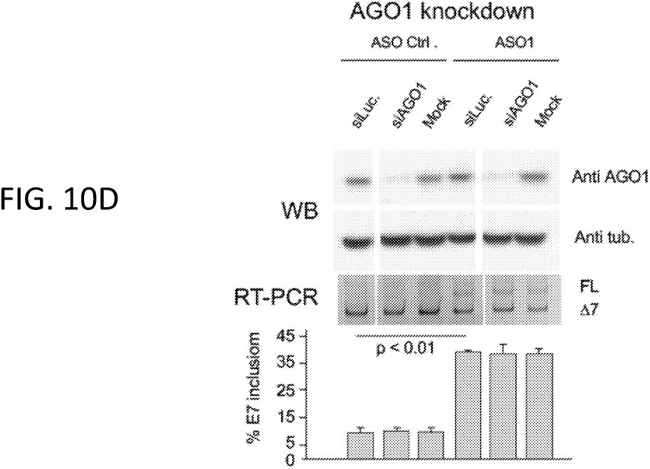

The observed synergistic effect was not merely the consequence of an increase in the pre-mRNA available to ASO1, because a similar synergistic effect can be seen at 10 times lower concentration of ASO1 (FIG. 5F). ChIP analysis, shown in FIG. 2A, showed that VPA promoted intragenic H3K9 acetylation along the entire SMN½ gene, and not just at the promoter. There was a conspicuous peak of acetylation more than 25 kb downstream of the promoter, around the E7 region, which can explain why E7 inclusion was upregulated, according to the kinetic-coupling mechanism of alternative splicing (Kornblihtt et al., Nat. Rev. Mol. Cell Biol. 14, 153-165, 2013). We ruled out the possibility that the HDAC inhibitors may be acting through modulation of SMN protein levels, which in turn might affect SMN2 E7 splicing, because overexpressing SMN did not alter the effect of ASO1 (FIG. 10A).

### Example 3

### Chromatin Effects of ASO1

Due to the fact that ASO1 is a single-stranded oligonucleotide that hybridizes with an intronic sequence at the pre-mRNA level. Thus, we evaluated whether ASO1 could have chromatin effects similar to those of siRNAs directed to intronic regions, as described in a proposed mechanism of TGS-AS (transcriptional gene silencing-regulated alternative splicing) (Alló et al., Nat. Struct. Mol. Biol. 16, 717-724, 2009; Schor et al., EMBO J. 32, 2264-2274, 2013). In TGS-AS, siRNAs targeting intronic sequences downstream of an alternative exon regulate the splicing of that exon by promoting silencing marks, such as H3K9me2 and H3K27me3, which in turn act as roadblocks to RNAPII elongation. This effect was only observed with siRNAs whose guide strand is complementary to the nascent mRNA (antisense), depends on Argonaute-1 (AGO1) and heterochromatic protein 1 (HP1), and is counterbalanced by factors that favor chromatin opening or transcriptional elongation.

Remarkably, ChIP analysis showed that transfection of HEK293T cells with ASO1 promoted extensive H3K9 dimethylation along the entire SMN½ gene, with peaks that reached an 8-fold increase at the promoter and the alternative E7 areas (FIG. 2B). Other gistone marks, like H3K27m3 or H3K9Ac, were not affected by ASO1 transfection (FIG. 10B). Most importantly, VPA not only reduced the H3K9me2 marks relative to the control but also completely abolished the ASO1 effect, confirming antagonistic roles of histone methylation and acetylation. The relevance of these findings is two-fold: first, they demonstrated unforeseen chromatin effects of an ASO on the deployment of histone marks at the targeted gene; and second, they showed that these effects can be abrogated by chromatin opening with HDAC inhibitors.

Next, whether RNAPII densities were affected. Transfection with ASO1 greatly increased total (FIG. 2C), P-Ser5 (FIG. 2D) and P-Ser2 (FIG. 2E) RNAPII densities at the promoter and at a distinct peak spanning about 6 kb beginning at and downstream of the ASO1 target site on the transcript, i.e., near the ASO1 target site on the transcript. In all three cases, the RNAPII peak was abolished when cells transfected with ASO1 were additionally treated with VPA. By promoting H3K9 methylation, ASO1 seemed to create a roadblock to RNAPII upstream of its target site. This roadblock appeared to equally affect the total and the two main phosphoisoforms of RNAPII, indicating that it acted as a general steric impediment, rather than regulating RNAPII phosphorylation. The increases in H3K9m2 and RNAPII at the promoter region, which does not have a target site of ASO1, appears to reflect looping between the two ends of the gene.

The chromatin effect of ASO1 did not appear to involve R-loop formation, because overexpression of RNase H, an enzyme that degrades RNA in RNA-DNA hybrids, did not affect ASO1′s effect on E7 inclusion (FIG. 10C). Moreover, ASO1′s effect was not altered by depleting AGO1 (FIG. 10D) as would be expected if the chromatin effect involved the same mechanism as that of siRNAs.

### Example 4

### Uncoupling the Two Opposite Roles of ASO1

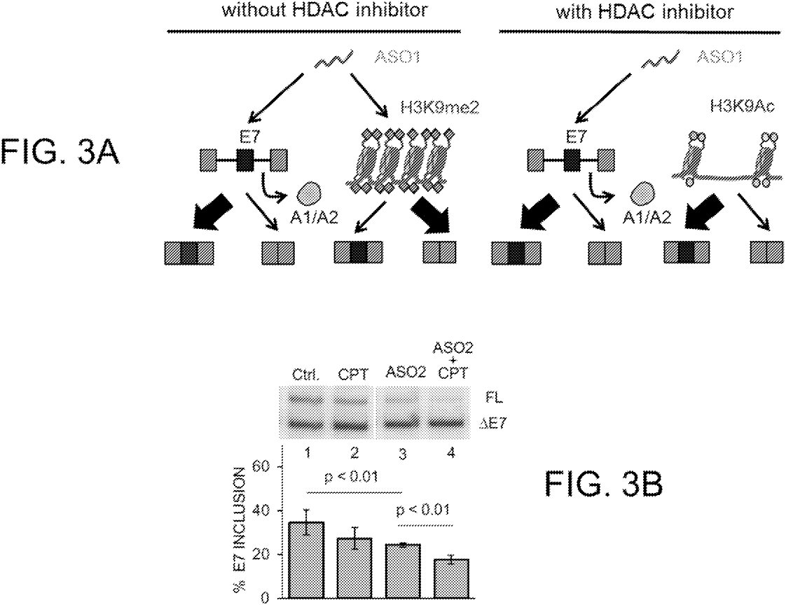

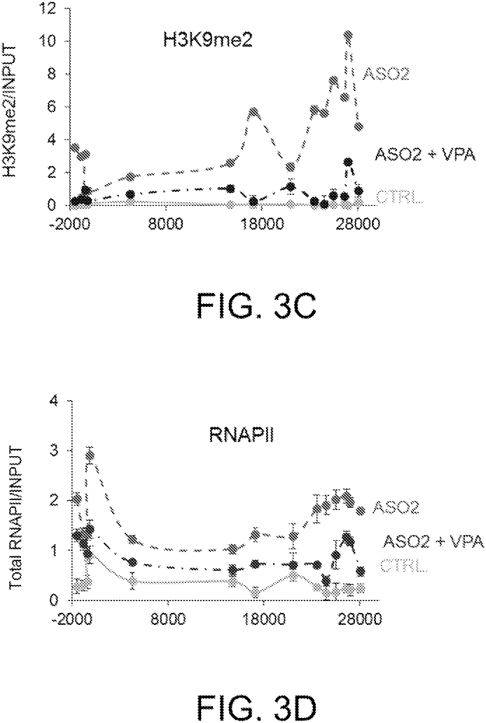

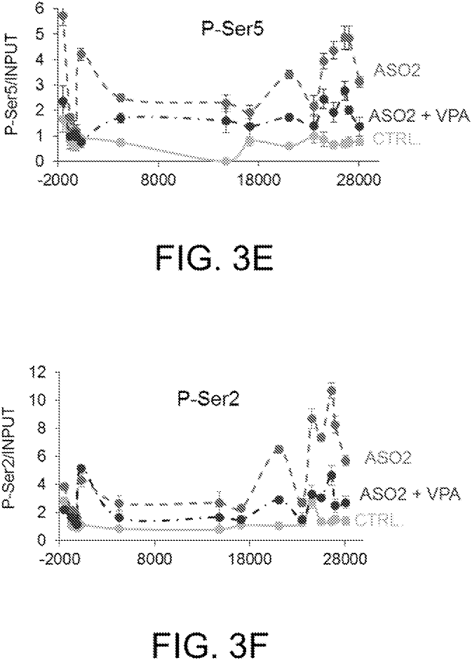

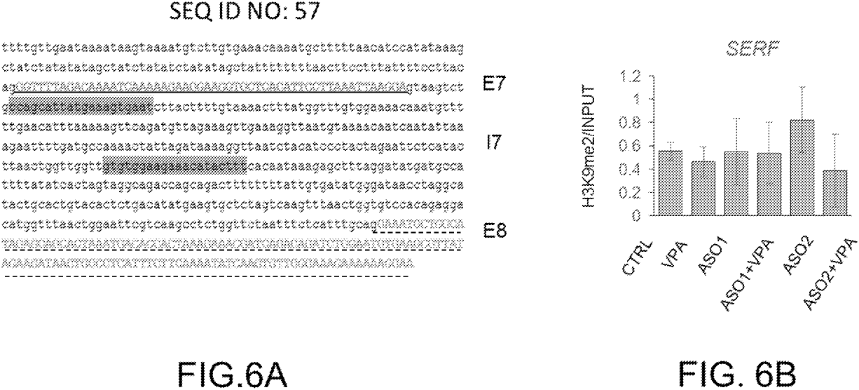

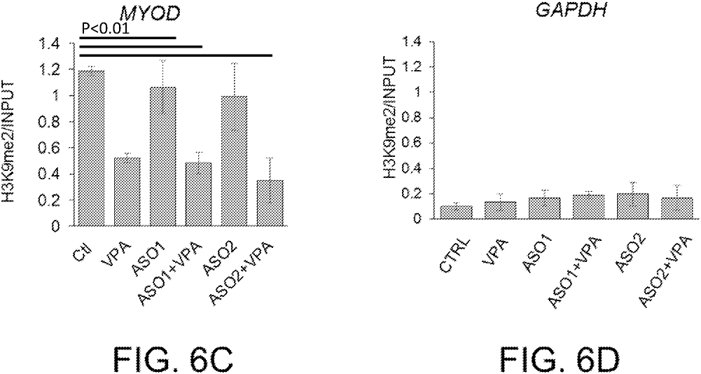

The results shown above indicate that in the absence of HDAC inhibitors, ASO1 had two opposing effects on E7 inclusion (FIG. 3A, left panel). ASO1 promoted inclusion by blocking hnRNPA1 and A2 binding to the pre-mRNA, but concomitantly resulted in a compact chromatin structure and a roadblock to elongation that in turn promoted E7 skipping. Although both effects co-exist, at high ASO1 concentrations, the chromatin effect was evidently surpassed by the hnRNPA1/A2 effect. On the other hand, in the presence of HDAC inhibitors (FIG. 3A, right panel), the chromatin-silencing effect was abrogated, and the levels of E7 inclusion increased further. To test the validity of this model, the effects of a second ASO, ASO2, whose target site is also located in intron 7, but downstream of the ASO1 target site were investigated (FIG. 6A). ASO2 bore no sequence identity with ASO1, and did not overlap hnRNPA1/A2 binding sites. Nevertheless, if it also displayed the chromatin effect, it should inhibit E7 inclusion.

The data in FIG. 3B showed that transfection of HEK293T cells with ASO2 inhibited SMN2 E7 inclusion as efficiently as the elongation inhibitor CPT, and that the combination of both treatments was stronger. Confirming the predicted chromatin and elongation effects, similarly to ASO1, ASO2 promoted H3K9 methylation (FIG. 3C) and higher total (FIG. 3D), P-Ser5 (FIG. 3E), and P-Ser2 (FIG. 3F) RNAPII densities, consistent with roadblocks to elongation that in all cases were abolished by treatment with VPA.

As specificity controls, neither ASO1 nor ASO2 promoted H3K9 dimethylation in a gene located in the same topologically-associated domain (TAD) as SMN2 (Lefebvre et al., Cell 80, 155-165, 1995) (FIG. 6B), or in genes located outside the SMN2 TAD with either high (FIG. 6C) or low (FIG. 6D) basal levels of H3K9 methylation.

These results provided a detailed mechanism for the cooperative effects of chromatin opening by HDAC inhibitors and ASOs designed to compete with splicing factors. The uncoupling experiments with ASO2 not only confirmed the model in FIG. 3A, but also highlighted the importance of taking into account chromatin effects when evaluating antisense therapeutic strategies. At saturating doses, nusinersen promoted nearly full inclusion of SMN2 E7 (Hua et al., Nature. 478, 123-126, 2011). Accordingly, the positive effects of HDAC inhibitors can be more critical in organs or at times in which the local ASO concentration is suboptimal,

### Example 5

### Combined Treatment in SMA Mice

Intrathecally-injected nusinersen is transiently present in blood (Chiriboga et al., Neurology 86, 890-897, 2016) and from where it can reach and act in peripheral tissues (Hua et al., Nature 478, 123-126, 2011). Accordingly, we assessed the systemic effects of the combined treatments of the present disclosure in a mouse model of severe SMA.

Mice have only one Smn gene, and homozygous mutants (Smn^{-/-}) are embryonic lethal (Hsieh-Li et al., Nat. Genet. 24, 66-70, 2000; Monani et al., Hum. Mol. Genet. 12; 333-339, 2000). Introduction of a human SMN2 transgene in an Smn^{-/-} background rescues embryonic lethality, displaying a range of phenotypes, depending on the SMN2 copy number (Hsieh-Li et al., Nat. Genet. 24, 66-70, 2000; Monani et al., Hum Mol Genet. 12; 333-339, 2000). Smn-null transgenic mice containing two copies of the SMN2 transgene, which develop a severe SMA-like phenotype with a mean survival of ~7-10 days were used (Hua et al., Nature 478, 123-126, 2011). Two consecutive low dose subcutaneous injections of ASO1 (18 µg/g) at P0 and P1 and/or one subcutaneous injection of VPA (10 µg/g) at P1 were administered as described above.

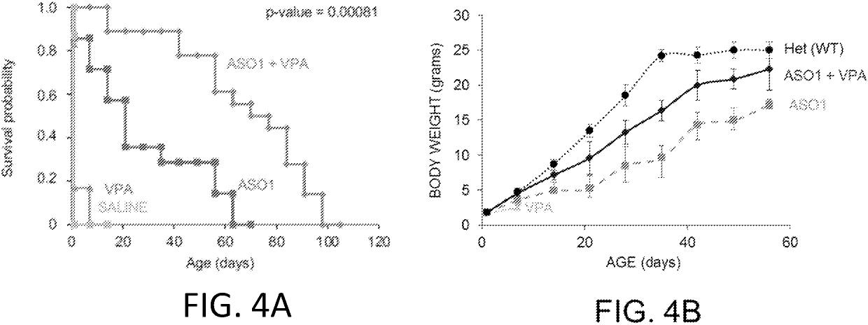

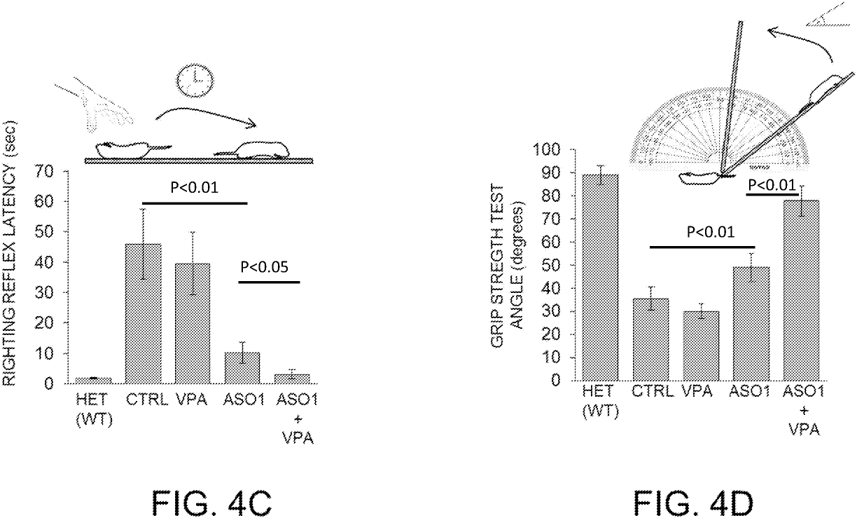

The data in FIG. 4A showed that mice injected with VPA alone died before P10, similarly to vehicle-treated controls. Survival was significantly extended with the suboptimal dose of ASO1 alone, but only 15% of the mice were still alive at P50. Co-injection of VPA with ASO1 increased survival by almost 5-fold, with 70% of the treated mice still alive at P50. Therefore, though VPA alone was found to be ineffective when administered as a monotherapy, when co-administered with the ASO1, a synergistic effect was observed.

The suboptimal dose of ASO1 alone extended median survival to ~20 days, while co-injection of VPA with ASO1 increased the median survival to ~70 days. All ASO1-alone-injected mice were dead at P62, whereas ~60% of those treated with ASO1 and VPA were still alive at P62. The difference between the two treatments is statistically significant (p = 0.00081).

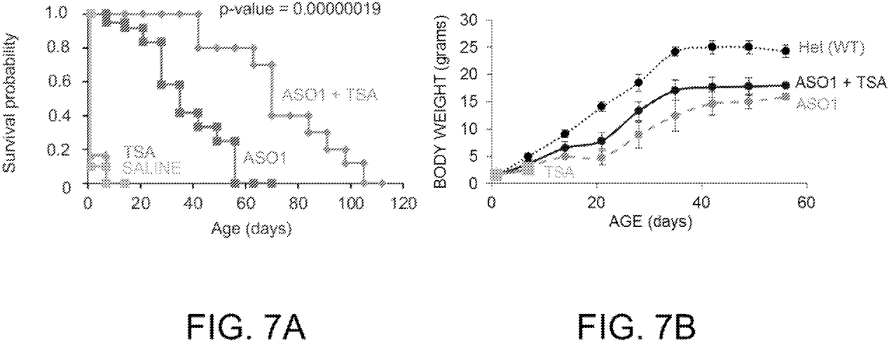

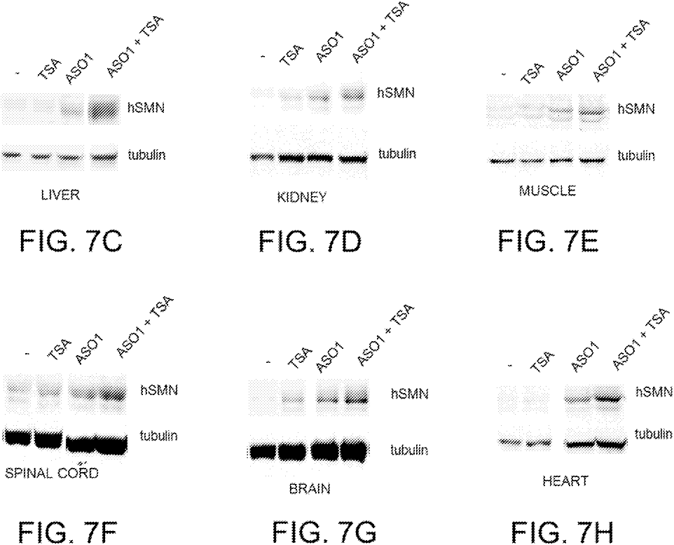

Body weight gain in the surviving mice was greatly improved by VPA plus ASO1 injection, compared to ASO1 alone (FIG. 4B). Similar results with a combined treatment of ASO1 and TSA (trichostatin A) were observed (FIG. 7A and FIG. 7B). Western blot analysis showed that the combined treatment greatly increased the levels of human SMN protein in liver, kidney, skeletal muscle, spinal cord, brain, and heart (FIGS. 7C, 7D, 7E, 7F, 7G, and 7H). The largest effect on SMN expression was in the liver (FIG. 7C), underscoring the role of this organ in SMA pathogenesis, in part as a source of circulating insulin-like growth factor 1 (Hua et al., Nature 478, 123-126, 2011).

To complement the survival and weight-gain data, two noninvasive neuromuscular function tests, appropriate for mouse pups, were performed. In the righting reflex test, the time it takes a mouse to right itself when placed on its back on a flat surface was measured (Feather-Schussler et al., 2016). Whereas wild-type mice righted immediately, untreated mutant mice took ~45 seconds. This delay was not significantly changed by VPA treatment, was greatly reduced by ASO1 treatment (~10 seconds). The delay was virtually eliminated by the combined treatment (FIG. 4C).

In the grip-strength test, the angle at which the pups fall from a tablet with a rough surface to which they hold on by their forelimbs was measured. The data in FIG. 4D showed that animals treated with both ASO1 and VPA were able to hold on at much higher inclination angles (~ 70°), compared with the untreated mutants (~35°) or with mice treated with VPA alone (~30°) or ASO1 alone (~45°).

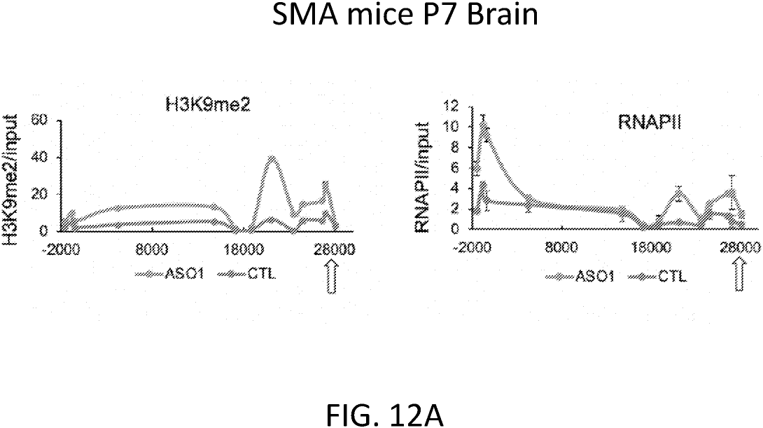

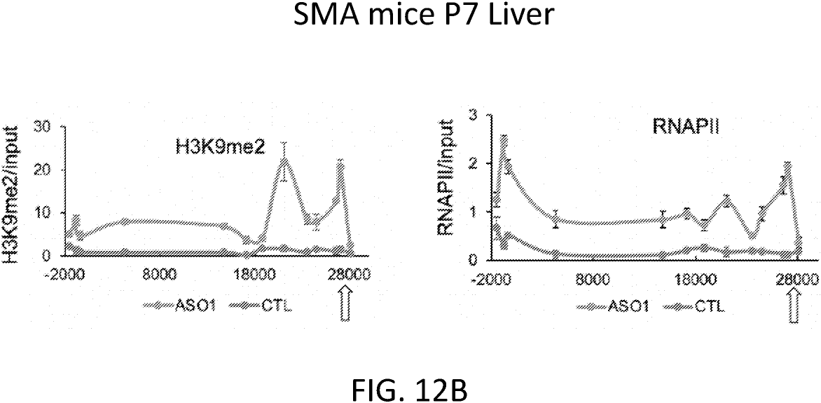

As in cultured cells, ASO1 injection into SMA mice also promoted higher H3K9 dimethylation and RNAPII accumulation on the SMN2 transgene in liver and brain (FIGS. 12A and 12B). This result indicated that the mechanism elucidated in cultured cells also underlied the survival and behavioral effects observed in mice.

Unlike ASO1, VPA has pleiotropic effects. However, VPA is currently used to treat several neurological disorders. The lack of an effect per se in SMA mice (FIGS. 4A and 4B) contrasting with the strong effect in improving ASO1′s therapeutic effects, indicates that optimal VPA dosing can be established in human patients with minimal potential toxicity.

The results presented herein indicate systemic administration of VPA can improvise the efficacy of nusinersen, particularly in peripheral tissues wherein nusinersen is present at low concentration, due to CSF clearance after intrathecal injection, contributing to its overall therapeutic effect.

### Example 6

### Genome-Wide Analysis

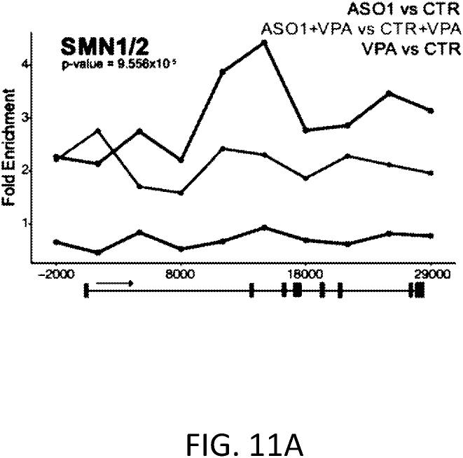

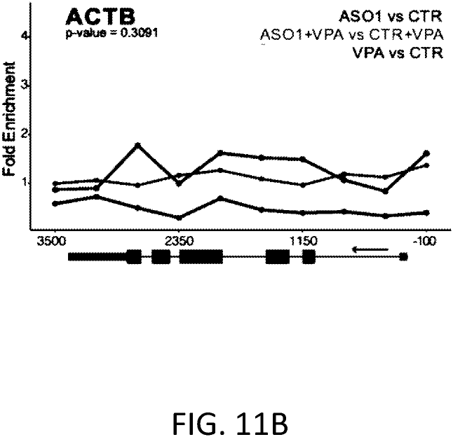

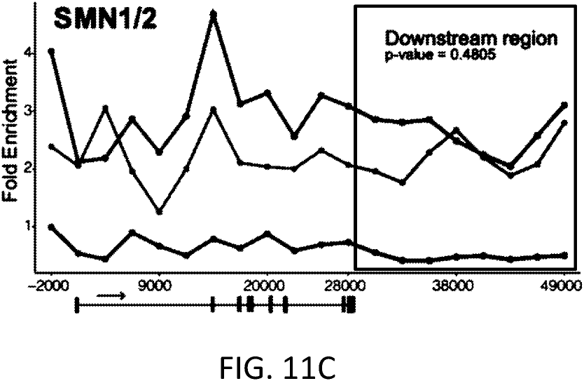

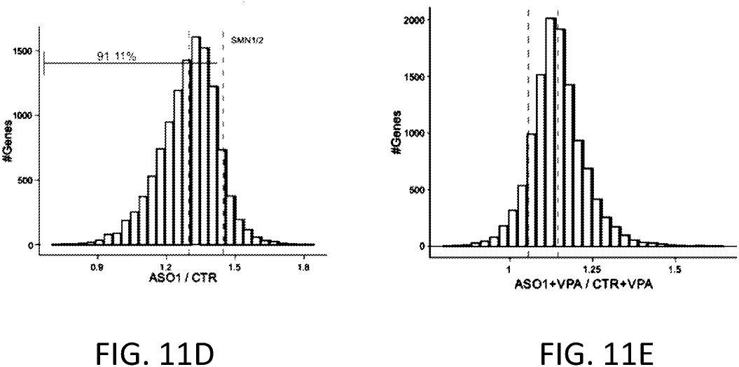

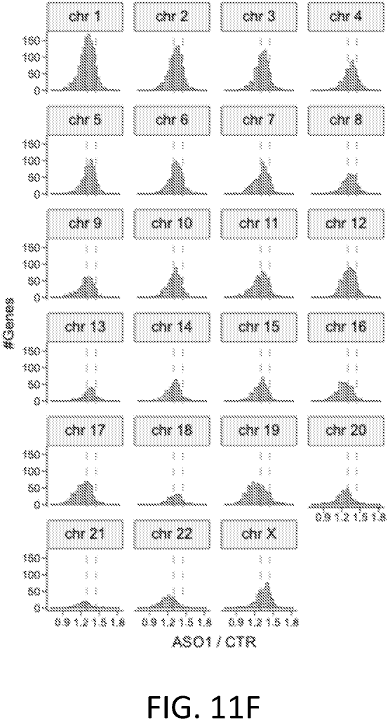

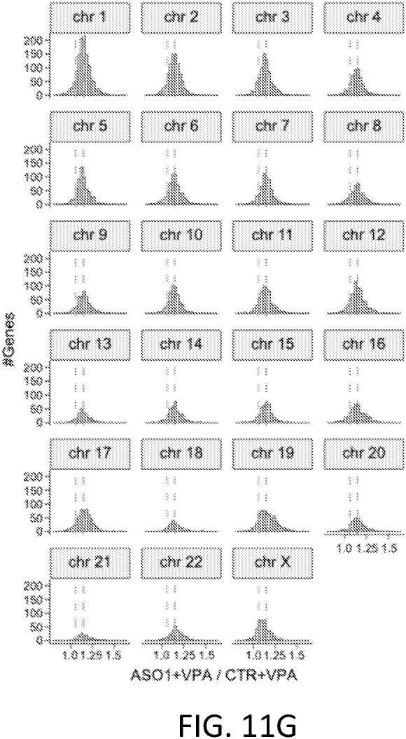

The effects of ASO1 and/or VPA on the deployment of the H3K9me2 mark throughout the genome by using ChIP-seq were analyzed. There was an enrichment of 44% for ASO1-treated cells compared with the control (FIG. 11A), in agreement with the ChIP-qPCR results (FIGS. 2A-2E). Conversely, when cells were treated with VPA, only a trace level of the methylation mark was detected, representing a decrease of 67%.

Importantly, the difference between ASO1 plus VPA and ASO1 alone was statistically significant (p = 9.556 × 10-5). As a control, the H3K9me2 mark over the ACTB gene (FIG. 11B) was measured, and no enrichment was detected for the same comparison (p = 0.3091). Additionally, the ChIP-seq signal about 20 kb upstream and downstream of the ASO1 binding site was quantified (FIG. 11C), revealing that ASO1′s effect and its reversion by VPA were limited to the SMN½ merged genes.

Next, whether ASO1-treated cells had enhanced H3K9me2 mark on other genes, was analyzed using a set of 11,738 protein-coding genes as a reference. Notably, the fold enrichment (ASO1/control) on the SMN½ genes was higher than the one observed for ~91% of the genes in the control set (FIG. 11D). Similarly, the reverting effect of VPA was higher than for the majority of the control genes (FIG. 11E) The approximately 9% of genes with higher ASO1/control may reflect the considerable background noise associated with ChIP-seq experiments, partial complementarity to ASO1 in gene transcripts other than SMN½, or indirect effects. Furthermore, ASO1-treatment and VPA reversion had larger effects for SMN than for other genes in each individual human chromosome (FIGS. 11F and 11G). These genome-wide analyses reinforced the evidence that the ASO1 chromatin effect on the SMN genes is highly specific.

What is claimed is:

1. A method for treating a neuromuscular disease or disorder comprising administering a therapeutically effective amount of a composition to a subject in need thereof wherein the composition comprises
(i) an antisense oligonucleotide (ASO) complementary to a nucleotide sequence within intron 7 of human SMN2 pre-mRNA; and
(ii) a subclinical dose of a histone deacetylase inhibitor.

2. A method for treating a neuromuscular disease or disorder comprising co-administering
(i) a therapeutically effective amount of an ASO complementary to a nucleotide sequence within intron 7 of human SMN2 pre-mRNA; and
(ii) a subclinical dose of a histone deacetylase inhibitor.

3. A method to increase the clinical efficacy of an ASO to a nucleotide sequence within intron 7 of human SMN2 pre-mRNA in the treatment of a neuromuscular disease or disorder comprising co-administering (i) a therapeutically effective amount of the ASO to a subject, and (ii) subclinical dose of a histone deacetylase inhibitor.

4. The method of any one of claims 1 to 3, wherein the neuromuscular disease or disorder is SMA.

5. The method of any one of claims 1 to 4, wherein the ASO is selected from the group consisting of ATTCACTTTCATAATGCTGG (ASO1) (SEQ ID NO:1), TGCTGGCAGACTTAC (SEQ ID NO:58), CATAATGCTGGCAGA (SEQ ID NO:59), TCATAATGCTGGCAG (SEQ ID NO:60), TTCATAATGCTGGCA (SEQ ID NO:61), TTTCATAATGCTGGC (SEQ ID NO:62), TCACTTTCATAATGCTGG (nusinersen) (SEQ ID NO:63), AGTAAGATTCACTTT (SEQ ID NO:64), CTTTCATAATGCTGG (SEQ ID NO:65), TCATAATGCTGG (SEQ ID NO:66), ACTTTCATAATGCTG (SEQ ID NO:67), TTCATAATGCTG (SEQ ID NO:68), CACTTTCATAATGCT (SEQ ID NO:69), TTTCATAATGCT (SEQ ID NO:70), TCACTTTCATAATGC (SEQ ID NO:71), CTTTCATAATGC (SEQ ID NO:72), TTCACTTTCATAATG (SEQ ID NO:73), ACTTTCATAATG (SEQ ID NO:74), ATTCACTTTCATAAT (SEQ ID NO:75), CACTTTCATAAT (SEQ ID NO:76), GATTCACTTTCATAA (SEQ ID NO:77), TCACTTTCATAA (SEQ ID NO:78), TTCACTTTCATA (SEQ ID NO:79), ATTCACTTTCAT (SEQ ID NO:80), or a combination thereof.

6. The method of any one of claims 1 to 4, wherein the ASO comprises a sequence which is at least about 70%, at least about 75%, at least about 80%, at least about 85%, at least about 90%, at least about 95%, at least about 96%, at least about 97%, at least about 98%, at least about 99%, or about 100% identical to ATTCACTTTCATAATGCTGG (ASO1) (SEQ ID NO:1), TGCTGGCAGACTTAC (SEQ ID NO:58), CATAATGCTGGCAGA (SEQ ID NO:59), TCATAATGCTGGCAG (SEQ ID NO:60), TTCATAATGCTGGCA (SEQ ID NO:61), TTTCATAATGCTGGC (SEQ ID NO:62), TCACTTTCATAATGCTGG (nusinersen) (SEQ ID NO:63), AGTAAGATTCACTTT (SEQ ID NO:64), CTTTCATAATGCTGG (SEQ ID NO:65), TCATAATGCTGG (SEQ ID NO:66), ACTTTCATAATGCTG (SEQ ID NO:67), TTCATAATGCTG (SEQ ID NO:68), CACTTTCATAATGCT (SEQ ID NO:69), TTTCATAATGCT (SEQ ID NO:70), TCACTTTCATAATGC (SEQ ID NO:71), CTTTCATAATGC (SEQ ID NO:72), TTCACTTTCATAATG (SEQ ID NO:73), ACTTTCATAATG (SEQ ID NO:74), ATTCACTTTCATAAT (SEQ ID NO:75), CACTTTCATAAT (SEQ ID NO:76), GATTCACTTTCATAA (SEQ ID NO:77), TCACTTTCATAA (SEQ ID NO:78), TTCACTTTCATA (SEQ ID NO:79), or ATTCACTTTCAT (SEQ ID NO:80).

7. The method of any one of claims 1 to 4, wherein the ASO is an ASO of SEQ ID NO: 1, nusinersen, a variant thereof, a derivative thereof, or a combination thereof.

8. The method of any one of claims 1 to 7, wherein the histone deacetylase inhibitor is valproic acid, trichostatin A, or a combination thereof.

9. The method of any one of claims 1 to 8, wherein (i) the ASO is administered at a dose lower than about 0.20 mg/kg, lower than about 0.19 mg/kg, lower than about 0.18 mg/kg, lower than about 0.17 mg/kg, lower than about 0.16 mg/kg, lower than about 0.15 mg/kg, lower than about 0.14 mg/kg, lower than about 0.13 mg/kg, lower than about 0.12 mg/kg, lower than about 0.11 mg/kg, lower than about 0.1 mg/kg, lower than about 0.09 mg/kg, lower than about 0.08 mg/kg, lower than about 0.07 mg/kg, lower than about 0.06 mg/kg, lower than about 0.05 mg/kg, lower than about 0.04 mg/kg, lower than about 0.03 mg/kg, lower than about 0.02 mg/kg, or lower than about 0.01 mg/kg per dose; and (ii) the histone deacetylase inhibitor is administered at a dose lower than about 15 mg/kg, lower than about 15 mg/kg, lower than about 13 mg/kg, lower than about 12 mg/kg, lower than about 11 mg/kg, lower than about 10 mg/kg, lower than about 9 mg/kg, lower than about 8 mg/kg, lower than about 7 mg/kg, lower than about 6 mg/kg, lower than about 5 mg/kg, lower than about 4 mg/kg, lower than about 3 mg/kg, lower than about 2 mg/kg, lower than about 1 mg/kg per dose.

10. The method of any one of claims 1 to 9, wherein (i) the ASO is administered at a dose lower than about 12 mg/dose, lower than about 11 mg/dose, lower than about 10 mg/dose, lower than about 9 mg/dose, lower than about 8 mg/dose, lower than about 7 mg/dose, lower than about 6 mg/dose, lower than about 5 mg/dose, lower than about 4 mg/dose, lower than about 3 mg/dose, lower than about 2 mg/dose, or lower than about 1 mg/dose; and (ii) the histone deacetylase inhibitor is administered at a dose lower than about 600 mg/dose, lower than about 550 mg/dose, lower than about 500 mg/dose, lower than about 550 mg/dose, lower than about 500 mg/dose, lower than about 450 mg/dose, lower than about 400 mg/dose, lower than about 350 mg/dose, lower than about 300 mg/dose, lower than about 250 mg/dose, lower than about 200 mg/dose, lower than about 175 mg/dose, lower than about 150 mg/dose, lower than about 125 mg/dose, lower than about 100 mg/dose, lower than about 90 mg/dose, lower than about 80 mg/dose, lower than about 70 mg/dose, lower than about 60 mg/dose, lower than about 50 mg/dose, lower than about 40 mg/dose, lower than about 30 mg/dose, lower than about 20 mg/dose, or lower than about 10 mg/dose.

11. The method of any one of claims 1 to 10, wherein (i) the ASO is administered at a dose lower than about 12 mg/dose/day, lower than about 11 mg/dose/day, lower than about 10 mg/dose/day, lower than about 9 mg/dose/day, lower than about 8 mg/dose/day, lower than about 7 mg/dose/day, lower than about 6 mg/dose/day, lower than about 5 mg/dose/day, lower than about 4 mg/dose/day, lower than about 3 mg/dose/day, lower than about 2 mg/dose/day, or lower than about 1 mg/dose/day; and (ii) the histone deacetylase inhibitor is administered at a dose lower than about 600 mg/dose/day, lower than about 550 mg/dose/day, lower than about 500 mg/dose/day, lower than about 550 mg/dose/day, lower than about 500 mg/dose/day, lower than about 450 mg/dose/day, lower than about 400 mg/dose/day, lower than about 350 mg/dose/day, lower than about 300 mg/dose/day, lower than about 250 mg/dose/day, lower than about 200 mg/dose/day, lower than about 175 mg/dose/day, lower than about 150 mg/dose/day, lower than about 125 mg/dose/day, lower than about 100 mg/dose/day, lower than about 90 mg/dose/day, lower than about 80 mg/dose/day, lower than about 70 mg/dose/day, lower than about 60 mg/dose/day, lower than about 50 mg/dose/day, lower than about 40 mg/dose/day, lower than about 30 mg/dose/day, lower than about 20 mg/dose/day, or lower than about 10 mg/dose/day.

12. The method of any one of claims 1 to 11, wherein (i) the ASO is administered at a dose of about 0.20 mg/kg, about 0.19 mg/kg, about 0.18 mg/kg, about 0.17 mg/kg, about 0.16 mg/kg, about 0.15 mg/kg, about 0.14 mg/kg, about 0.13 mg/kg, about 0.12 mg/kg, about 0.11 mg/kg, about 0.1 mg/kg, about 0.09 mg/kg, about 0.08 mg/kg, about 0.07 mg/kg, about 0.06 mg/kg, about 0.05 mg/kg, about 0.04 mg/kg, about 0.03 mg/kg, about 0.02 mg/kg, or about 0.01 mg/kg per dose; and (ii) the histone deacetylase inhibitor is administered at a dose about 15 mg/kg, about 15 mg/kg, about 13 mg/kg, about 12 mg/kg, about 11 mg/kg, about 10 mg/kg, about 9 mg/kg, about 8 mg/kg, about 7 mg/kg, about 6 mg/kg, about 5 mg/kg, about 4 mg/kg, about 3 mg/kg, about 2 mg/kg, about 1 mg/kg per dose.

13. The method of any one of claims 1 to 12, wherein (i) the ASO is administered at a dose of about 12 mg/dose, about 11 mg/dose, about 10 mg/dose, about 9 mg/dose, about 8 mg/dose, about 7 mg/dose, about 6 mg/dose, about 5 mg/dose, about 4 mg/dose, about 3 mg/dose, about 2 mg/dose, or about 1 mg/dose; and (ii) the histone deacetylase inhibitor is administered at a dose about 600 mg/dose, about 550 mg/dose, about 500 mg/dose, about 550 mg/dose, about 500 mg/dose, about 450 mg/dose, about 400 mg/dose, about 350 mg/dose, about 300 mg/dose, about 250 mg/dose, about 200 mg/dose, about 175 mg/dose, about 150 mg/dose, about 125 mg/dose, about 100 mg/dose, about 90 mg/dose, about 80 mg/dose, about 70 mg/dose, about 60 mg/dose, about 50 mg/dose, about 40 mg/dose, about 30 mg/dose, about 20 mg/dose, or about 10 mg/dose.

14. The method of any one of claims 1 to 13, wherein (i) the ASO is administered at a dose of about 12 mg/dose/day, about 11 mg/dose/day, about 10 mg/dose/day, about 9 mg/dose/day, about 8 mg/dose/day, about 7 mg/dose/day, about 6 mg/dose/day, about 5 mg/dose/day, about 4 mg/dose/day, about 3 mg/dose/day, about 2 mg/dose/day, or about 1 mg/dose/day; and (ii) the histone deacetylase inhibitor is administered at a dose about 600 mg/dose/day, about 550 mg/dose/day, about 500 mg/dose/day, about 550 mg/dose/day, about 500 mg/dose/day, about 450 mg/dose/day, about 400 mg/dose/day, about 350 mg/dose/day, about 300 mg/dose/day, about 250 mg/dose/day, about 200 mg/dose/day, about 175 mg/dose/day, about 150 mg/dose/day, about 125 mg/dose/day, about 100 mg/dose/day, about 90 mg/dose/day, about 80 mg/dose/day, about 70 mg/dose/day, about 60 mg/dose/day, about 50 mg/dose/day, about 40 mg/dose/day, about 30 mg/dose/day, about 20 mg/dose/day, or about 10 mg/dose/day.

15. The method of any one of claims 1 to 14, wherein the administration of the ASO complementary to a nucleotide sequence within intron 7 of human SMN2 pre-mRNA; and the subclinical dose of a histone deacetylate inhibitor results in an increase in inclusion of exon 7 of SMN2, an increase in the expression of SMN2 protein with exon 7, a decrease in the expression of SMN2 protein without exon 7, or any combination thereof.

16. The method of any one of claims 1 to 15, wherein the administration of the ASO complementary to a nucleotide sequence within intron 7 of human SMN2 pre-mRNA; and the subclinical dose of a histone deacetylate inhibitor results in an increase in time of survival, increase in body mass, increase in muscle coordination, improvement in neuromuscular function, or any combination thereof.

17. The method of any one of claims 1 to 15, wherein the ASO and the subclinical dose of a histone deacetylase inhibitor are administered together.

18. The method of any one of claims 1 to 15, wherein the ASO and the subclinical dose of a histone deacetylase inhibitor are administered separately.

19. The method of any one of claims 1 to 18, wherein the ASO and the subclinical dose of a histone deacetylase inhibitor are administered at the same time.

20. The method of any one of claims 1 to 18, wherein the ASO is administered prior to the administration of the subclinical dose of histone deacetylase inhibitor.

21. The method of any one of claims 1 to 18, wherein the subclinical dose of histone deacetylase inhibitor is administered prior to the administration of the ASO.

22. The method of any one of claim 1 to 20, wherein the ASO administered intrathecally or intravenously.

23. The method of any one of claims 1 to 22, wherein the histone deacetylase inhibitor administered orally or intravenously.

24. The method of any one of claims 1 to 23, wherein the ASO is a gapmer, a mixmer, or a totalmer.

25. The method of any one of claims 1 to 24 wherein the ASO comprises one or more nucleoside analogs.

26. The method of claim 26, wherein one or more of the nucleoside analogs comprises a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; or bicyclic nucleoside analog.

27. The method of claim 25 or 26, wherein one or more of the nucleoside analogs is a sugar modified nucleoside.

28. The method of claim 27, wherein the sugar modified nucleoside is an affinity enhancing 2′ sugar modified nucleoside.

29. The method of any one of claims 25 to 28, wherein one or more of the nucleoside analogs comprises a nucleoside comprising a bicyclic sugar.

30. The method of any one of claims 25 to 29, wherein one or more of the nucleoside analogs comprises an LNA.

31. The method of any one of claim 25 to 30, wherein one or more of the nucleotide analogs is selected from the group consisting of constrained ethyl nucleoside (cEt), 2′,4′-constrained 2′-O-methoxyethyl (cMOE), α-L-LNA, β-D-LNA, 2′-O,4′-C-ethylene-bridged nucleic acids (ENA), amino-LNA, oxy-LNA, thio-LNA, and any combination thereof.

32. The method of any one of claims 1 to 31, wherein the ASO comprises one or more 5′-methyl-cytosine nucleobases.

33. The method of any one of claims 1 to 32, wherein the ASO is from 15 to 25 nucleotides in length.

34. The method of any one of claims 1 to 33, wherein the ASO comprises one or more modified internucleoside linkages.

35. The method of claim 34, wherein the one or more modified internucleoside linkages is a phosphorothioate linkage.

36. The method of claims 34 or 35, wherein at least 75%, at least 80%, at least 85%, at least 90%, at least 95%, or 100% of internucleoside linkages are modified.

37. The method of claim 36, wherein each of the internucleoside linkages in the ASO is a phosphorothioate linkage.


SEQUENCE LISTING (86 sequences)

SEQ ID NO: 1 - DNA, 20 bp, Artificial Sequence
  ASO1
attcactttcataatgctgg

SEQ ID NO: 2 - DNA, 20 bp, Artificial Sequence
  Synthetic sequence
aaagtatgtttcttccacac

SEQ ID NO: 3 - RNA, 20 nt, Artificial Sequence
  Synthetic sequence
auucacuuucauaaugcugg

SEQ ID NO: 4 - DNA, 23 bp, Artificial Sequence
  Synthetic sequence
aagtgagaactccaggtctcctg

SEQ ID NO: 5 - DNA, 24 bp, Artificial Sequence
  Synthetic sequence
tctgatcgtttctttagtggtgtc

SEQ ID NO: 6 - DNA, 21 bp, Artificial Sequence
  Synthetic sequence
cgggtttgctatggcgatgag

SEQ ID NO: 7 - DNA, 20 bp, Artificial Sequence
  Synthetic sequence
ggctgcggaaggagagttgg

SEQ ID NO: 8 - DNA, 23 bp, Artificial Sequence
  Synthetic sequence
ccttccatattccagttctcttg

SEQ ID NO: 9 - DNA, 22 bp, Artificial Sequence
  Synthetic sequence
tacctgtaacgcttcacattcc

SEQ ID NO: 10 - DNA, 22 bp, Artificial Sequence
  Synthetic sequence
caaatgtgggagggcgataacc

SEQ ID NO: 11 - DNA, 21 bp, Artificial Sequence
  Synthetic sequence
ttctgggagcggaacagtacg

SEQ ID NO: 12 - DNA, 19 bp, Artificial Sequence
  Synthetic sequence
ggctcactacaacctcctc

SEQ ID NO: 13 - DNA, 23 bp, Artificial Sequence
  Synthetic sequence
ttcagattattctcctccattcc

SEQ ID NO: 14 - DNA, 25 bp, Artificial Sequence
  Synthetic sequence
tccttacagggttttagacaaaatc

SEQ ID NO: 15 - DNA, 23 bp, Artificial Sequence
  Synthetic sequence
cataatgctggcagacttactcc

SEQ ID NO: 16 - DNA, 23 bp, Artificial Sequence
  Synthetic sequence
acatataagccatttagcaaccc

SEQ ID NO: 17 - DNA, 21 bp, Artificial Sequence
  Synthetic sequence
gaactcctgacctcgtaatcc

SEQ ID NO: 18 - DNA, 22 bp, Artificial Sequence
  Synthetic sequence
gagaccagcctacacaatatgc

SEQ ID NO: 19 - DNA, 18 bp, Artificial Sequence
  Synthetic sequence
ccgcctcaacctcccaag

SEQ ID NO: 20 - DNA, 25 bp, Artificial Sequence
  Synthetic sequence
ccgaaaccccgtctctactaaatac

SEQ ID NO: 21 - DNA, 20 bp, Artificial Sequence
  Synthetic sequence
aagcgatcctcccacctcag

SEQ ID NO: 22 - DNA, 23 bp, Artificial Sequence
  Synthetic sequence
aatgtagtatggttctgtgtctc

SEQ ID NO: 23 - DNA, 20 bp, Artificial Sequence
  Synthetic sequence
gtttgctcaaggtagtctgg

SEQ ID NO: 24 - DNA, 21 bp, Artificial Sequence
  Synthetic sequence
tttatctccctcccgctattc

SEQ ID NO: 25 - DNA, 24 bp, Artificial Sequence
  Synthetic sequence
aatctctttgagtcttagtttccc

SEQ ID NO: 26 - DNA, 20 bp, Artificial Sequence
  Synthetic sequence
tctcttgatgatgctgatgc

SEQ ID NO: 27 - DNA, 22 bp, Artificial Sequence
  Synthetic sequence
agatgctgagtgattacttacc

SEQ ID NO: 28 - DNA, 19 bp, Artificial Sequence
  Synthetic sequence
gcaaggcaaggcattacag

SEQ ID NO: 29 - DNA, 21 bp, Artificial Sequence
  Synthetic sequence
cctgagcaatagagtgagacc

SEQ ID NO: 30 - DNA, 18 bp, Artificial Sequence
  Synthetic sequence
cactgtgcctagcctgag

SEQ ID NO: 31 - DNA, 20 bp, Artificial Sequence
  Synthetic sequence
ggatcacttgagacctggag

SEQ ID NO: 32 - DNA, 20 bp, Artificial Sequence
  Synthetic sequence
actggttggttgtgtggaag

SEQ ID NO: 33 - DNA, 19 bp, Artificial Sequence
  Synthetic sequence
agtctgctggtctgcctac

SEQ ID NO: 34 - DNA, 19 bp, Artificial Sequence
  Synthetic sequence
acagtgggtgggcattcag

SEQ ID NO: 35 - DNA, 18 bp, Artificial Sequence
  Synthetic sequence
gagcggtggcgacagtag

SEQ ID NO: 36 - DNA, 18 bp, Artificial Sequence
  Synthetic sequence
tctctgccgcttgggttg

SEQ ID NO: 37 - DNA, 21 bp, Artificial Sequence
  Synthetic sequence
catagaagtcgtccgttgtgg

SEQ ID NO: 38 - DNA, 20 bp, Artificial Sequence
  Synthetic sequence
ctctctcccatcccttctcc

SEQ ID NO: 39 - DNA, 18 bp, Artificial Sequence
  Synthetic sequence
ttgccaagttgcctgtcc

SEQ ID NO: 40 - DNA, 19 bp, Artificial Sequence
  Synthetic sequence
atgaatgggcagccgttag

SEQ ID NO: 41 - DNA, 18 bp, Artificial Sequence
  Synthetic sequence
tcgctccacctgacttcc

SEQ ID NO: 42 - DNA, 24 bp, Artificial Sequence
  Synthetic sequence
gcatcgttaaagtctctcttcacc

SEQ ID NO: 43 - DNA, 22 bp, Artificial Sequence
  Synthetic sequence
ctcctcaggctctcatcagttg

SEQ ID NO: 44 - DNA, 23 bp, Artificial Sequence
  Synthetic sequence
tggcattaaagaagacactgaag

SEQ ID NO: 45 - DNA, 22 bp, Artificial Sequence
  Synthetic sequence
tcaaaggttacaaaggcaaagc

SEQ ID NO: 46 - DNA, 23 bp, Artificial Sequence
  Synthetic sequence
aagtgagaactccaggtctcctg

SEQ ID NO: 47 - DNA, 25 bp, Artificial Sequence
  Synthetic sequence
cttctgagtcttgggcatgtcagtg

SEQ ID NO: 48 - DNA, 24 bp, Artificial Sequence
  Synthetic sequence
gaatgggaagcttgtcatcaacgg

SEQ ID NO: 49 - DNA, 24 bp, Artificial Sequence
  Synthetic sequence
ccgttcagctctgggatgaccttg

SEQ ID NO: 50 - DNA, 20 bp, Artificial Sequence
  Synthetic sequence
ctgaagcttttggctttgag

SEQ ID NO: 51 - DNA, 19 bp, Artificial Sequence
  Synthetic sequence
ccgctgccaaattctttgg

SEQ ID NO: 52 - DNA, 30 bp, Artificial Sequence
  Synthetic sequence
ttgtctagagagcatggagggccatgtcaa

SEQ ID NO: 53 - DNA, 30 bp, Artificial Sequence
  Synthetic sequence
ccactcctctgtgacactttagccctccga

SEQ ID NO: 54 - DNA, 21 bp, Artificial Sequence
  Synthetic sequence
ataacaccaccactcttactc

SEQ ID NO: 55 - DNA, 21 bp, Artificial Sequence
  Synthetic sequence
gtagccgtgatgccattgtca

SEQ ID NO: 56 - DNA, 21 bp, Artificial Sequence
  Synthetic sequence
agcctgaagaacgagatcagc

SEQ ID NO: 57 - RNA, 8 nt, Artificial Sequence
  Synthetic sequence
ccagcauu

SEQ ID NO: 58 - DNA, 15 bp, Artificial Sequence
  Synthetic sequence
tgctggcagacttac

SEQ ID NO: 59 - DNA, 15 bp, Artificial Sequence
  Synthetic sequence
cataatgctggcaga

SEQ ID NO: 60 - DNA, 15 bp, Artificial Sequence
  Synthetic sequence
tcataatgctggcag

SEQ ID NO: 61 - DNA, 15 bp, Artificial Sequence
  Synthetic sequence
ttcataatgctggca

SEQ ID NO: 62 - DNA, 15 bp, Artificial Sequence
  Synthetic sequence
tttcataatgctggc

SEQ ID NO: 63 - DNA, 18 bp, Artificial Sequence
  Nusinersen
tcactttcataatgctgg

SEQ ID NO: 64 - DNA, 15 bp, Artificial Sequence
  Synthetic sequence
agtaagattcacttt

SEQ ID NO: 65 - DNA, 15 bp, Artificial Sequence
  Synthetic sequence
ctttcataatgctgg

SEQ ID NO: 66 - DNA, 12 bp, Artificial Sequence
  Synthetic sequence
tcataatgctgg

SEQ ID NO: 67 - DNA, 15 bp, Artificial Sequence
  Synthetic sequence
actttcataatgctg

SEQ ID NO: 68 - DNA, 12 bp, Artificial Sequence
  Synthetic sequence
ttcataatgctg

SEQ ID NO: 69 - DNA, 15 bp, Artificial Sequence
  Synthetic sequence
cactttcataatgct

SEQ ID NO: 70 - DNA, 12 bp, Artificial Sequence
  Synthetic sequence
tttcataatgct

SEQ ID NO: 71 - DNA, 15 bp, Artificial Sequence
  Synthetic sequence
tcactttcataatgc

SEQ ID NO: 72 - DNA, 12 bp, Artificial Sequence
  Synthetic sequence
ctttcataatgc

SEQ ID NO: 73 - DNA, 15 bp, Artificial Sequence
  Synthetic sequence
ttcactttcataatg

SEQ ID NO: 74 - DNA, 12 bp, Artificial Sequence
  Synthetic sequence
actttcataatg

SEQ ID NO: 75 - DNA, 15 bp, Artificial Sequence
  Synthetic sequence
attcactttcataat

SEQ ID NO: 76 - DNA, 12 bp, Artificial Sequence
  Synthetic sequence
cactttcataat

SEQ ID NO: 77 - DNA, 15 bp, Artificial Sequence
  Synthetic sequence
gattcactttcataa

SEQ ID NO: 78 - DNA, 12 bp, Artificial Sequence
  Synthetic sequence
tcactttcataa

SEQ ID NO: 79 - DNA, 12 bp, Artificial Sequence
  Synthetic sequence
ttcactttcata

SEQ ID NO: 80 - DNA, 12 bp, Artificial Sequence
  Synthetic sequence
attcactttcat

SEQ ID NO: 81 - RNA, 15 nt, Artificial Sequence
  Synthetic sequence
ccagcauuaugaaag

SEQ ID NO: 82 - RNA, 18 nt, Artificial Sequence
  Synthetic sequence
ccagcauuaugaaaguga

SEQ ID NO: 83 - RNA, 20 nt, Artificial Sequence
  Synthetic sequence
ccagcauuaugaaagugaau

SEQ ID NO: 84 - DNA, 15 bp, Artificial Sequence
  Synthetic sequence
  misc_feature (6)..(10): Wherein n can be any nucleotide
ccagcnnnnngaaag

SEQ ID NO: 85 - DNA, 22 bp, Artificial Sequence
  Synthetic sequence
gctcaggtgatccaactgtctc

SEQ ID NO: 86 - DNA, 19 bp, Artificial Sequence
  Synthetic sequence
cgtggtggctcaggctagg